SEQUENCE LISTING (703 sequences)

SEQ ID NO: 1 - DNA, 29 bp, Homo sapiens
  misc_feature (15)..(15): n is c or g
agcacagctccgagntacttgtcatatta

SEQ ID NO: 2 - DNA, 29 bp, Homo sapiens
  misc_feature (15)..(15): n is c or t
tttattcataattantaagccaaaaatat

SEQ ID NO: 3 - DNA, 29 bp, Homo sapiens
  misc_feature (15)..(15): n is g or t
gttttgtgagacctnggcaataatcgagg

SEQ ID NO: 4 - DNA, 29 bp, Homo sapiens
  misc_feature (15)..(15): n is a or g
acctgtgcaacagtngtgggtagttttca

SEQ ID NO: 5 - DNA, 29 bp, Homo sapiens
  misc_feature (15)..(15): n is c or t
taaactaactcaaantaacacattaataa

SEQ ID NO: 6 - DNA, 29 bp, Homo sapiens
  misc_feature (15)..(15): n is a or t
tagccaaaaacttancattgtataagtat

SEQ ID NO: 7 - DNA, 29 bp, Homo sapiens
  misc_feature (15)..(15): n is a or g
ctgtccctctttccnctgagagtaaaagt

SEQ ID NO: 8 - DNA, 29 bp, Homo sapiens
  misc_feature (15)..(15): n is a or g
ctgtccctctttccnctgagagtaaaagt

SEQ ID NO: 9 - DNA, 29 bp, Homo sapiens
  misc_feature (15)..(15): n is g or t
tttttctttaagttntgcatgcgcatatg

SEQ ID NO: 10 - DNA, 29 bp, Homo sapiens
  misc_feature (15)..(15): n is c or t
gatgtcacaggcacnagtcaaatttgagt

SEQ ID NO: 11 - DNA, 29 bp, Homo sapiens
  misc_feature (15)..(15): n is a or g
tattggcaatgtctntaaagacacagctc

SEQ ID NO: 12 - DNA, 29 bp, Homo sapiens
  misc_feature (15)..(15): n is a or g
gacatagggagcacnttgcgaaggtaaca

SEQ ID NO: 13 - DNA, 29 bp, Homo sapiens
  misc_feature (15)..(15): n is a or g
ttcagatgagttttntaattcagttggag

SEQ ID NO: 14 - DNA, 29 bp, Homo sapiens
  misc_feature (15)..(15): n is c or g
tgcatgagtttaaantccatgactgttat

SEQ ID NO: 15 - DNA, 29 bp, Homo sapiens
  misc_feature (15)..(15): n is a or g
atgttttaaccccantgcagtaagatgct

SEQ ID NO: 16 - DNA, 29 bp, Homo sapiens
  misc_feature (15)..(15): n is c or t
cacagtctgccaggncctgggcatgacag

SEQ ID NO: 17 - DNA, 29 bp, Homo sapiens
  misc_feature (15)..(15): n is c or t
cacagtctgccaggncctgggcatgacag

SEQ ID NO: 18 - DNA, 29 bp, Homo sapiens
  misc_feature (15)..(15): n is a or g
tgggtgccactcccntgaagtggtgaagg

SEQ ID NO: 19 - DNA, 29 bp, Homo sapiens
  misc_feature (15)..(15): n is a or g
tgggctctgaacccncagggaagttaggt

SEQ ID NO: 20 - DNA, 29 bp, Homo sapiens
  misc_feature (15)..(15): n is c or t
ctagacagttctggncttcaggcctgcgg

SEQ ID NO: 21 - DNA, 29 bp, Homo sapiens
  misc_feature (15)..(15): n is a or g
tctctggtgtgaacnttttcaagtgaaca

SEQ ID NO: 22 - DNA, 29 bp, Homo sapiens
  misc_feature (15)..(15): n is c or g
taccatacaaagctnatgtggtagttctt

SEQ ID NO: 23 - DNA, 29 bp, Homo sapiens
  misc_feature (15)..(15): n is a or g
catattgtacttctngtgtgtctgattaa

SEQ ID NO: 24 - DNA, 29 bp, Homo sapiens
  misc_feature (15)..(15): n is a or c
ggcagtgagtcttangtctgcatagggct

SEQ ID NO: 25 - DNA, 29 bp, Homo sapiens
  misc_feature (15)..(15): n is c or g
ttacagacatagatnccgtcttaagcaaa

SEQ ID NO: 26 - DNA, 29 bp, Homo sapiens
  misc_feature (15)..(15): n is a or t
atcctctgtcaatanttaccatgttatct

SEQ ID NO: 27 - DNA, 29 bp, Homo sapiens
  misc_feature (15)..(15): n is a or g
accctcaggtagatnatgtatgtaatgta

SEQ ID NO: 28 - DNA, 29 bp, Homo sapiens
  misc_feature (15)..(15): n is c or g
ttgaaagtgtcaatntattgtataatcag

SEQ ID NO: 29 - DNA, 29 bp, Homo sapiens
  misc_feature (15)..(15): n is a or c
cttggttatgaaatnatccttctcctaaa

SEQ ID NO: 30 - DNA, 29 bp, Homo sapiens
  misc_feature (15)..(15): n is a or g
agaaaatgagctgangataatgaatggac

SEQ ID NO: 31 - DNA, 29 bp, Homo sapiens
  misc_feature (15)..(15): n is a or g
ccaactcacacattncagctaattccaac

SEQ ID NO: 32 - DNA, 29 bp, Homo sapiens
  misc_feature (15)..(15): n is c or g
gctgaatttgtcctnaactgttttctcct

SEQ ID NO: 33 - DNA, 29 bp, Homo sapiens
  misc_feature (15)..(15): n is c or t
ataatcgcattgcanattcacaggtacct

SEQ ID NO: 34 - DNA, 29 bp, Homo sapiens
  misc_feature (15)..(15): n is a or g
ctttcactttccctntcgatacttcatat

SEQ ID NO: 35 - DNA, 29 bp, Homo sapiens
  misc_feature (15)..(15): n is c or t
gaagatctctcttcnatatccaggattat

SEQ ID NO: 36 - DNA, 29 bp, Homo sapiens
  misc_feature (15)..(15): n is c or t
catgaactgttaagntaatttgctttttg

SEQ ID NO: 37 - DNA, 29 bp, Homo sapiens
  misc_feature (15)..(15): n is a or t
aatggacgattctcngtccactgttatgt

SEQ ID NO: 38 - DNA, 29 bp, Homo sapiens
  misc_feature (15)..(15): n is c or t
tagaaactctacttntgttaaacttaagc

SEQ ID NO: 39 - DNA, 29 bp, Homo sapiens
  misc_feature (15)..(15): n is c or t
aaaagaaattatctntgtaatcactcatg

SEQ ID NO: 40 - DNA, 29 bp, Homo sapiens
  misc_feature (15)..(15): n is a or c
aagagccaggttcanacaacatattaatg

SEQ ID NO: 41 - DNA, 29 bp, Homo sapiens
  misc_feature (15)..(15): n is c or t
ttgcaaacattggtntcattattttttga

SEQ ID NO: 42 - DNA, 29 bp, Homo sapiens
  misc_feature (15)..(15): n is g or t
tctgtgagcattgangctttcttttaata

SEQ ID NO: 43 - DNA, 29 bp, Homo sapiens
  misc_feature (15)..(15): n is a or g
ccttttaaatgtaanagtagactaaaaga

SEQ ID NO: 44 - DNA, 29 bp, Homo sapiens
  misc_feature (15)..(15): n is c or t
ttgcttagagcaccntaaggacacctggg

SEQ ID NO: 45 - DNA, 29 bp, Homo sapiens
  misc_feature (15)..(15): n is c or t
agaatggcctatacntttgaaattattga

SEQ ID NO: 46 - DNA, 29 bp, Homo sapiens
  misc_feature (15)..(15): n is a or g
tttaccttgagtgtntaaaatactccatg

SEQ ID NO: 47 - DNA, 29 bp, Homo sapiens
  misc_feature (15)..(15): n is c or t
gggaggtatctaaantgaacatagcatac

SEQ ID NO: 48 - DNA, 29 bp, Homo sapiens
  misc_feature (15)..(15): n is c or t
taggttcaactgatncatccttttgaatt

SEQ ID NO: 49 - DNA, 29 bp, Homo sapiens
  misc_feature (15)..(15): n is a or g
ctgagcatttgctcnaaggccattacaaa

SEQ ID NO: 50 - DNA, 29 bp, Homo sapiens
  misc_feature (15)..(15): n is c or t
tttgggccctgttangaacacctgtagtg

SEQ ID NO: 51 - DNA, 29 bp, Homo sapiens
  misc_feature (15)..(15): n is g or t
acagaactgtactangagaaaacattttc

SEQ ID NO: 52 - DNA, 29 bp, Homo sapiens
  misc_feature (15)..(15): n is g or t
acagaactgtactangagaaaacattttc

SEQ ID NO: 53 - DNA, 29 bp, Homo sapiens
  misc_feature (15)..(15): n is a or g
cttcaaggctattanggcagatacaaata

SEQ ID NO: 54 - DNA, 29 bp, Homo sapiens
  misc_feature (15)..(15): n is c or t
ctcattcaatctttntggtcttctatact

SEQ ID NO: 55 - DNA, 29 bp, Homo sapiens
  misc_feature (15)..(15): n is c or t
agttgcagcctgtancctagtcaacatag

SEQ ID NO: 56 - DNA, 29 bp, Homo sapiens
  misc_feature (15)..(15): n is c or t
tatagctattgatanactgtatatatgac

SEQ ID NO: 57 - DNA, 29 bp, Homo sapiens
  misc_feature (15)..(15): n is a or g
gaggaaactctgaanttttagtcaaaact

SEQ ID NO: 58 - DNA, 29 bp, Homo sapiens
  misc_feature (15)..(15): n is c or t
tcactaatttaacantaactttgcattca

SEQ ID NO: 59 - DNA, 29 bp, Homo sapiens
  misc_feature (15)..(15): n is a or g
gtcatttggcattanggtcttttccagca

SEQ ID NO: 60 - DNA, 29 bp, Homo sapiens
  misc_feature (15)..(15): n is c or t
taagcttgagtcccnagtctgattttctc

SEQ ID NO: 61 - DNA, 29 bp, Homo sapiens
  misc_feature (15)..(15): n is c or t
ttaactcccatattnaagctatttatact

SEQ ID NO: 62 - DNA, 29 bp, Homo sapiens
  misc_feature (15)..(15): n is c or t
ttaactcccatattnaagctatttatact

SEQ ID NO: 63 - DNA, 29 bp, Homo sapiens
  misc_feature (15)..(15): n is c or t
ttctacaatgtacangataaacatactaa

SEQ ID NO: 64 - DNA, 29 bp, Homo sapiens
  misc_feature (15)..(15): n is a or g
ggaaacgagtgcctntaaaagtccataaa

SEQ ID NO: 65 - DNA, 29 bp, Homo sapiens
  misc_feature (15)..(15): n is a or g
agatgctaccttaanagaggtaaatgaag

SEQ ID NO: 66 - DNA, 29 bp, Homo sapiens
  misc_feature (15)..(15): n is a or g
aatcctctgcaatcntaagtgcaaaacct

SEQ ID NO: 67 - DNA, 29 bp, Homo sapiens
  misc_feature (15)..(15): n is a or g
ttgcttaatgaaatntaagtggacgaatg

SEQ ID NO: 68 - DNA, 29 bp, Homo sapiens
  misc_feature (15)..(15): n is c or g
tattatattttcacnaatttctgtttatt

SEQ ID NO: 69 - DNA, 29 bp, Homo sapiens
  misc_feature (15)..(15): n is c or t
tgaaagtattttaangagacttccattgc

SEQ ID NO: 70 - DNA, 29 bp, Homo sapiens
  misc_feature (15)..(15): n is a or g
ggccataataattcntgagctgaaattac

SEQ ID NO: 71 - DNA, 29 bp, Homo sapiens
  misc_feature (15)..(15): n is c or t
taaaaacagttgaantgaaaaagaaaagg

SEQ ID NO: 72 - DNA, 29 bp, Homo sapiens
  misc_feature (15)..(15): n is a or t
tttttaatatgctcnaatcaatgcaatat

SEQ ID NO: 73 - DNA, 29 bp, Homo sapiens
  misc_feature (15)..(15): n is c or t
aatggcaaagcctcnaaaaagagaggtaa

SEQ ID NO: 74 - DNA, 29 bp, Homo sapiens
  misc_feature (15)..(15): n is c or t
aattgcagctctcanggtggaaataacag

SEQ ID NO: 75 - DNA, 29 bp, Homo sapiens
  misc_feature (15)..(15): n is a or g
cgcgcctgtgtaagngccttatcgccctc

SEQ ID NO: 76 - DNA, 29 bp, Homo sapiens
  misc_feature (15)..(15): n is c or g
atatagtcattttantgtttttttttgtg

SEQ ID NO: 77 - DNA, 29 bp, Homo sapiens
  misc_feature (15)..(15): n is c or g
atatagtcattttantgtttttttttgtg

SEQ ID NO: 78 - DNA, 29 bp, Homo sapiens
  misc_feature (15)..(15): n is a or g
gtctagtaaattccntaagtaagactttt

SEQ ID NO: 79 - DNA, 29 bp, Homo sapiens
  misc_feature (15)..(15): n is c or g
ttttctatctctttnaagattactgtaaa

SEQ ID NO: 80 - DNA, 29 bp, Homo sapiens
  misc_feature (15)..(15): n is a or c
tcgtaatgcctgtanataatttaacatga

SEQ ID NO: 81 - DNA, 29 bp, Homo sapiens
  misc_feature (15)..(15): n is a or g
gtttttcatccatanggtgtgtaccttcc

SEQ ID NO: 82 - DNA, 29 bp, Homo sapiens
  misc_feature (15)..(15): n is a or g
atgcaccgaaggtanaggataccaggttc

SEQ ID NO: 83 - DNA, 29 bp, Homo sapiens
  misc_feature (15)..(15): n is c or t
caatgcagtacccantgaaatttcctcat

SEQ ID NO: 84 - DNA, 29 bp, Homo sapiens
  misc_feature (15)..(15): n is c or g
aggcagaattctggnaaatgggtcgagag

SEQ ID NO: 85 - DNA, 29 bp, Homo sapiens
  misc_feature (15)..(15): n is c or t
aaaggctgctcaagngaagactctacata

SEQ ID NO: 86 - DNA, 29 bp, Homo sapiens
  misc_feature (15)..(15): n is g or t
atagttaaattgttncctgcaatgtcaag

SEQ ID NO: 87 - DNA, 29 bp, Homo sapiens
  misc_feature (15)..(15): n is a or g
gtaactcaagcattnacccacattcattt

SEQ ID NO: 88 - DNA, 29 bp, Homo sapiens
  misc_feature (15)..(15): n is c or t
tgaatgtactatganagaaaatattacca

SEQ ID NO: 89 - DNA, 29 bp, Homo sapiens
  misc_feature (15)..(15): n is c or g
aaaattttatcccanaatggaaatcatta

SEQ ID NO: 90 - DNA, 29 bp, Homo sapiens
  misc_feature (15)..(15): n is c or t
aagttaagtgaactntgaatttgtcagtc

SEQ ID NO: 91 - DNA, 29 bp, Homo sapiens
  misc_feature (15)..(15): n is g or t
tttggtagtttctgnaattttgtgtattt

SEQ ID NO: 92 - DNA, 29 bp, Homo sapiens
  misc_feature (15)..(15): n is c or t
ataaccacacttatngattagttcattta

SEQ ID NO: 93 - DNA, 29 bp, Homo sapiens
  misc_feature (15)..(15): n is c or t
aatccataagattgntaaatacatataag

SEQ ID NO: 94 - DNA, 29 bp, Homo sapiens
  misc_feature (15)..(15): n is a or g
taaagaaaaaaaatnctgtcctaagaaat

SEQ ID NO: 95 - DNA, 29 bp, Homo sapiens
  misc_feature (15)..(15): n is a or g
gcctgcagatagtanatactaaagcatct

SEQ ID NO: 96 - DNA, 29 bp, Homo sapiens
  misc_feature (15)..(15): n is c or t
tgactgtccaactangtcactgggggaac

SEQ ID NO: 97 - DNA, 29 bp, Homo sapiens
  misc_feature (15)..(15): n is c or t
tgactgtccaactangtcactgggggaac

SEQ ID NO: 98 - DNA, 29 bp, Homo sapiens
  misc_feature (15)..(15): n is c or t
catctgtgaaaggcngttactaaagaaga

SEQ ID NO: 99 - DNA, 29 bp, Homo sapiens
  misc_feature (15)..(15): n is a or g
tcaggagctactggnctgaggttcgattg

SEQ ID NO: 100 - DNA, 29 bp, Homo sapiens
  misc_feature (15)..(15): n is a or g
aatgaatcaacgatngaaactcgggcatg

SEQ ID NO: 101 - DNA, 29 bp, Homo sapiens
  misc_feature (15)..(15): n is a or g
aatccacaggagctnaactatacggactc

SEQ ID NO: 102 - DNA, 29 bp, Homo sapiens
  misc_feature (15)..(15): n is c or t
gtgttatggcagaanttgacattatcctt

SEQ ID NO: 103 - DNA, 29 bp, Homo sapiens
  misc_feature (15)..(15): n is a or g
ctagatttgaataantacttgatttgcat

SEQ ID NO: 104 - DNA, 29 bp, Homo sapiens
  misc_feature (15)..(15): n is c or t
aaacattctagatanatgcatcaaaacag

SEQ ID NO: 105 - DNA, 29 bp, Homo sapiens
  misc_feature (15)..(15): n is a or g
agacactgttcatcntaaaatacaggttt

SEQ ID NO: 106 - DNA, 29 bp, Homo sapiens
  misc_feature (15)..(15): n is a or g
aaatttaaaagcaantgatgacaactagc

SEQ ID NO: 107 - DNA, 29 bp, Homo sapiens
  misc_feature (15)..(15): n is a or t
aagagagatctgagntggggagatcagtc

SEQ ID NO: 108 - DNA, 29 bp, Homo sapiens
  misc_feature (15)..(15): n is a or g
tttcatcatattaantgtaatagtttccc

SEQ ID NO: 109 - DNA, 29 bp, Homo sapiens
  misc_feature (15)..(15): n is g or t
gggcactgtagtctncaatccctgacaat

SEQ ID NO: 110 - DNA, 29 bp, Homo sapiens
  misc_feature (15)..(15): n is c or g
tgggcgtcctggctnaggtgaaacagaaa

SEQ ID NO: 111 - DNA, 29 bp, Homo sapiens
  misc_feature (15)..(15): n is c or t
tggctcagaaaatanttacttaaacactt

SEQ ID NO: 112 - DNA, 29 bp, Homo sapiens
  misc_feature (15)..(15): n is a or g
ttattgagcagtganttgggtcaataact

SEQ ID NO: 113 - DNA, 29 bp, Homo sapiens
  misc_feature (15)..(15): n is a or g
agaattgagatatangtaaaaactatgca

SEQ ID NO: 114 - DNA, 29 bp, Homo sapiens
  misc_feature (15)..(15): n is c or g
ttccaccatacatantgaggaggatgtgc

SEQ ID NO: 115 - DNA, 29 bp, Homo sapiens
  misc_feature (15)..(15): n is a or g
agccgtcaagcaacntctaacttggaaga

SEQ ID NO: 116 - DNA, 29 bp, Homo sapiens
  misc_feature (15)..(15): n is c or g
ggtcttctaaaaacntgtggtgtcttttc

SEQ ID NO: 117 - DNA, 29 bp, Homo sapiens
  misc_feature (15)..(15): n is c or t
ttatttttctgagtncactgtttggaaat

SEQ ID NO: 118 - DNA, 29 bp, Homo sapiens
  misc_feature (15)..(15): n is a or g
agaatgactctgtgncaaaacactgtgat

SEQ ID NO: 119 - DNA, 29 bp, Homo sapiens
  misc_feature (15)..(15): n is a or g
agaaccatattgcanaagatcaggttgta

SEQ ID NO: 120 - DNA, 29 bp, Homo sapiens
  misc_feature (15)..(15): n is a or g
gagacaagtaacacnttgagaagctggat

SEQ ID NO: 121 - DNA, 29 bp, Homo sapiens
  misc_feature (15)..(15): n is c or g
ttagagaatcctagnaagaacattttgtg

SEQ ID NO: 122 - DNA, 29 bp, Homo sapiens
  misc_feature (15)..(15): n is c or t
atttaaattttatcngattggtgatatta

SEQ ID NO: 123 - DNA, 29 bp, Homo sapiens
  misc_feature (15)..(15): n is c or t
cgtgaatcctgttanggcctcagctcagg

SEQ ID NO: 124 - DNA, 29 bp, Homo sapiens
  misc_feature (15)..(15): n is c or t
ttcacacatcctccnaggtctttcaatgc

SEQ ID NO: 125 - DNA, 29 bp, Homo sapiens
  misc_feature (15)..(15): n is a or g
tttttcttaaaacangttataaaactcag

SEQ ID NO: 126 - DNA, 29 bp, Homo sapiens
  misc_feature (15)..(15): n is c or t
aatgttaatagttantccattagataaag

SEQ ID NO: 127 - DNA, 29 bp, Homo sapiens
  misc_feature (15)..(15): n is c or g
tcagggttctcttancaagaaacgagggg

SEQ ID NO: 128 - DNA, 29 bp, Homo sapiens
  misc_feature (15)..(15): n is g or t
atctgagaatcgttncctctttttaaatt

SEQ ID NO: 129 - DNA, 29 bp, Homo sapiens
  misc_feature (15)..(15): n is c or t
aaagcataataaatnagacatgtatctgc

SEQ ID NO: 130 - DNA, 29 bp, Homo sapiens
  misc_feature (15)..(15): n is c or g
aacacaggagggttngctttttcagagaa

SEQ ID NO: 131 - DNA, 29 bp, Homo sapiens
  misc_feature (15)..(15): n is c or t
agagtccttacctantgtattactttgct

SEQ ID NO: 132 - DNA, 29 bp, Homo sapiens
  misc_feature (15)..(15): n is c or t
tctactttacagcantctttccagatgtt

SEQ ID NO: 133 - DNA, 29 bp, Homo sapiens
  misc_feature (15)..(15): n is a or g
aaaatgatgagtacntatagacattaaat

SEQ ID NO: 134 - DNA, 29 bp, Homo sapiens
  misc_feature (15)..(15): n is a or g
agcctctaacccatntttttgtgttcagc

SEQ ID NO: 135 - DNA, 29 bp, Homo sapiens
  misc_feature (15)..(15): n is a or g
gttttcctctgcccntaatgactacgtat

SEQ ID NO: 136 - DNA, 29 bp, Homo sapiens
  misc_feature (15)..(15): n is c or t
gctgcataggattancatatcagtctata

SEQ ID NO: 137 - DNA, 29 bp, Homo sapiens
  misc_feature (15)..(15): n is a or g
attttaaatgcccanttttctataagaat

SEQ ID NO: 138 - DNA, 29 bp, Homo sapiens
  misc_feature (15)..(15): n is a or g
acttagcattctgcngcttatctaaatca

SEQ ID NO: 139 - DNA, 29 bp, Homo sapiens
  misc_feature (15)..(15): n is c or t
gatctcaaaggaggntggacggactgggg

SEQ ID NO: 140 - DNA, 29 bp, Homo sapiens
  misc_feature (15)..(15): n is c or t
agaatttaatagcangagatttgaaattt

SEQ ID NO: 141 - DNA, 29 bp, Homo sapiens
  misc_feature (15)..(15): n is a or g
gacaaccataggcanaggatggcaaggag

SEQ ID NO: 142 - DNA, 29 bp, Homo sapiens
  misc_feature (15)..(15): n is a or g
tgaaatgtcattganattaaaaagaaaaa

SEQ ID NO: 143 - DNA, 29 bp, Homo sapiens
  misc_feature (15)..(15): n is a or c
ctcacactttatatngtctttcaaaccta

SEQ ID NO: 144 - DNA, 29 bp, Homo sapiens
  misc_feature (15)..(15): n is c or g
catcacagttacagnccttctgaaaacag

SEQ ID NO: 145 - DNA, 29 bp, Homo sapiens
  misc_feature (15)..(15): n is c or t
gtggaataggtaaancactgtcaaataga

SEQ ID NO: 146 - DNA, 29 bp, Homo sapiens
  misc_feature (15)..(15): n is c or g
attatagacatatcncggattagaaggtg

SEQ ID NO: 147 - DNA, 29 bp, Homo sapiens
  misc_feature (15)..(15): n is c or t
ctaaatggagagccngattctcattattc

SEQ ID NO: 148 - DNA, 29 bp, Homo sapiens
  misc_feature (15)..(15): n is c or g
cgattgccccaaagnattcatcgaactac

SEQ ID NO: 149 - DNA, 29 bp, Homo sapiens
  misc_feature (15)..(15): n is a or g
atgagttttggctgnaagttacagaaact

SEQ ID NO: 150 - DNA, 29 bp, Homo sapiens
  misc_feature (15)..(15): n is c or t
ctatgactgtcatangtcaaaacaggatt

SEQ ID NO: 151 - DNA, 29 bp, Homo sapiens
  misc_feature (15)..(15): n is a or g
tgcagcagttctccngggactgtatgaag

SEQ ID NO: 152 - DNA, 29 bp, Homo sapiens
  misc_feature (15)..(15): n is a or g
tgcagcagttctccngggactgtatgaag

SEQ ID NO: 153 - DNA, 29 bp, Homo sapiens
  misc_feature (15)..(15): n is c or t
ttgcaaaaagtagtnaatgactccaggaa

SEQ ID NO: 154 - DNA, 29 bp, Homo sapiens
  misc_feature (15)..(15): n is a or g
ccttattcttctctntttcacagtatcta

SEQ ID NO: 155 - DNA, 29 bp, Homo sapiens
  misc_feature (15)..(15): n is a or g
aataatgacctgaantttctctgtagcca

SEQ ID NO: 156 - DNA, 29 bp, Homo sapiens
  misc_feature (15)..(15): n is a or g
tcttctctgttgacnattgttaatattag

SEQ ID NO: 157 - DNA, 29 bp, Homo sapiens
  misc_feature (15)..(15): n is a or g
gaaattgtttaaggngttaggtttactgg

SEQ ID NO: 158 - DNA, 29 bp, Homo sapiens
  misc_feature (15)..(15): n is c or t
atttcccgaggggangtgtctccaactgc

SEQ ID NO: 159 - DNA, 29 bp, Homo sapiens
  misc_feature (15)..(15): n is c or g
tattctatgcagganatttgcatattttg

SEQ ID NO: 160 - DNA, 29 bp, Homo sapiens
  misc_feature (15)..(15): n is c or t
ttgttggcgttgaanctggcaggaaaaag

SEQ ID NO: 161 - DNA, 29 bp, Homo sapiens
  misc_feature (15)..(15): n is g or t
gctctccattttaantttcagagctatcc

SEQ ID NO: 162 - DNA, 29 bp, Homo sapiens
  misc_feature (15)..(15): n is a or g
aactaatttgagtgnttattatggggatt

SEQ ID NO: 163 - DNA, 29 bp, Homo sapiens
  misc_feature (15)..(15): n is c or t
tggcctgaggctacntcaggttccagatt

SEQ ID NO: 164 - DNA, 29 bp, Homo sapiens
  misc_feature (15)..(15): n is a or g
cagttcccagatgtnatagtacttgtttt

SEQ ID NO: 165 - DNA, 29 bp, Homo sapiens
  misc_feature (15)..(15): n is a or g
cttaactggaagtcntaatgatggaattt

SEQ ID NO: 166 - DNA, 29 bp, Homo sapiens
  misc_feature (15)..(15): n is c or g
ttagtaaaagcccanttcttaatagttga

SEQ ID NO: 167 - DNA, 29 bp, Homo sapiens
  misc_feature (15)..(15): n is a or t
tcctgaaaaaatcantgacgattatggaa

SEQ ID NO: 168 - DNA, 29 bp, Homo sapiens
  misc_feature (15)..(15): n is a or g
acctctctattaatntgaaaaattgatca

SEQ ID NO: 169 - DNA, 29 bp, Homo sapiens
  misc_feature (15)..(15): n is g or t
atgtttaccaaattntattcctttgaaca

SEQ ID NO: 170 - DNA, 29 bp, Homo sapiens
  misc_feature (15)..(15): n is c or t
attcaattatttaangttagatgaatcat

SEQ ID NO: 171 - DNA, 29 bp, Homo sapiens
  misc_feature (15)..(15): n is c or t
tctcagagcaattgngccttcaaatgtca

SEQ ID NO: 172 - DNA, 29 bp, Homo sapiens
  misc_feature (15)..(15): n is c or t
tataaactgttcctnagttcatagtcttt

SEQ ID NO: 173 - DNA, 29 bp, Homo sapiens
  misc_feature (15)..(15): n is a or g
atagagaacactgantcttttcctgggag

SEQ ID NO: 174 - DNA, 29 bp, Homo sapiens
  misc_feature (15)..(15): n is g or t
ctcagaagtatattntctcacttgttcta

SEQ ID NO: 175 - DNA, 29 bp, Homo sapiens
  misc_feature (15)..(15): n is a or g
acacatttttagaangtcttgaatccttg

SEQ ID NO: 176 - DNA, 29 bp, Homo sapiens
  misc_feature (15)..(15): n is c or t
cccagcatttattangattcaccaaataa

SEQ ID NO: 177 - DNA, 29 bp, Homo sapiens
  misc_feature (15)..(15): n is c or t
aactcctactgtaanagctttcttgggaa

SEQ ID NO: 178 - DNA, 29 bp, Homo sapiens
  misc_feature (15)..(15): n is c or t
caatattttatcttncggggctgaaagaa

SEQ ID NO: 179 - DNA, 29 bp, Homo sapiens
  misc_feature (15)..(15): n is a or g
actaacagatctacntttccctttctacc

SEQ ID NO: 180 - DNA, 29 bp, Homo sapiens
  misc_feature (15)..(15): n is c or t
gcagagtcatgtctnttcctgctcagtga

SEQ ID NO: 181 - DNA, 29 bp, Homo sapiens
  misc_feature (15)..(15): n is a or c
ttggcatgagactcngtgagatgaagttg

SEQ ID NO: 182 - DNA, 29 bp, Homo sapiens
  misc_feature (15)..(15): n is c or t
agctaaaatcgtgantgaaacgtccattc

SEQ ID NO: 183 - DNA, 29 bp, Homo sapiens
  misc_feature (15)..(15): n is c or t
agactgtctactgcnaaagtccctttgaa

SEQ ID NO: 184 - DNA, 29 bp, Homo sapiens
  misc_feature (15)..(15): n is c or t
ttaatatttaatcanacagggtctcctat

SEQ ID NO: 185 - DNA, 29 bp, Homo sapiens
  misc_feature (15)..(15): n is a or g
gcatctgttcggagncctgatgacacaga

SEQ ID NO: 186 - DNA, 29 bp, Homo sapiens
  misc_feature (15)..(15): n is a or g
ttacctatgtctcgntttatttactacga

SEQ ID NO: 187 - DNA, 29 bp, Homo sapiens
  misc_feature (15)..(15): n is c or g
aatgtgtaggccagngatgtttttttaac

SEQ ID NO: 188 - DNA, 29 bp, Homo sapiens
  misc_feature (15)..(15): n is a or g
gaggaaggagttcangactcacgtgatgc

SEQ ID NO: 189 - DNA, 29 bp, Homo sapiens
  misc_feature (15)..(15): n is c or g
ctgaaaagggtgacnctgaaaaagcacag

SEQ ID NO: 190 - DNA, 29 bp, Homo sapiens
  misc_feature (15)..(15): n is c or t
ctgtttccgaaaaanagctttttctcctg

SEQ ID NO: 191 - DNA, 29 bp, Homo sapiens
  misc_feature (15)..(15): n is a or g
aaagagttaatgtgngtaaagcatttaga

SEQ ID NO: 192 - DNA, 29 bp, Homo sapiens
  misc_feature (15)..(15): n is c or t
agtaactaagcagcntacctgttttggtt

SEQ ID NO: 193 - DNA, 29 bp, Homo sapiens
  misc_feature (15)..(15): n is c or g
taatgcccattcatnaaagtgacagagac

SEQ ID NO: 194 - DNA, 29 bp, Homo sapiens
  misc_feature (15)..(15): n is a or g
tgttccagtgtcctntgtgttatcctgga

SEQ ID NO: 195 - DNA, 29 bp, Homo sapiens
  misc_feature (15)..(15): n is c or t
tattcaaattagttncacatatttatttt

SEQ ID NO: 196 - DNA, 29 bp, Homo sapiens
  misc_feature (15)..(15): n is c or g
ggccagggctctgcncacaaagctaaact

SEQ ID NO: 197 - DNA, 29 bp, Homo sapiens
  misc_feature (15)..(15): n is a or g
aaaaataaaataatnaggctgtgcacagt

SEQ ID NO: 198 - DNA, 29 bp, Homo sapiens
  misc_feature (15)..(15): n is a or g
aactattagaatgtnctgccatactagta

SEQ ID NO: 199 - DNA, 29 bp, Homo sapiens
  misc_feature (15)..(15): n is a or g
tcactgtcattatantaactaatactgtc

SEQ ID NO: 200 - DNA, 29 bp, Homo sapiens
  misc_feature (15)..(15): n is c or t
cactctttttgatgnactactacaattat

SEQ ID NO: 201 - DNA, 29 bp, Homo sapiens
  misc_feature (15)..(15): n is a or t
cattgaaattggcancagtgctggaaagt

SEQ ID NO: 202 - DNA, 29 bp, Homo sapiens
  misc_feature (15)..(15): n is a or g
ttttctttcatggtntcatattctgtcta

SEQ ID NO: 203 - DNA, 29 bp, Homo sapiens
  misc_feature (15)..(15): n is g or t
atacaacacaaatcntttttagagaatta

SEQ ID NO: 204 - DNA, 29 bp, Homo sapiens
  misc_feature (15)..(15): n is c or t
ctaatcaatcttatnactggtccttgaga

SEQ ID NO: 205 - DNA, 29 bp, Homo sapiens
  misc_feature (15)..(15): n is g or t
ataacagaaacaaangcccaggaatggag

SEQ ID NO: 206 - DNA, 29 bp, Homo sapiens
  misc_feature (15)..(15): n is a or g
caccttgcacaacanttagcttttcacag

SEQ ID NO: 207 - DNA, 29 bp, Homo sapiens
  misc_feature (15)..(15): n is a or g
gcactgtgtggtganttgccaggtgtgtg

SEQ ID NO: 208 - DNA, 29 bp, Homo sapiens
  misc_feature (15)..(15): n is c or t
tggggaaaaacatgnatgtgactttgtct

SEQ ID NO: 209 - DNA, 29 bp, Homo sapiens
  misc_feature (15)..(15): n is c or t
cttctaaaaatgaangtaaccacttgcaa

SEQ ID NO: 210 - DNA, 29 bp, Homo sapiens
  misc_feature (15)..(15): n is g or t
atgaagagaggtacnaaacctgtagcctg

SEQ ID NO: 211 - DNA, 29 bp, Homo sapiens
  misc_feature (15)..(15): n is g or t
gttaagattaatttnggacccattaaact

SEQ ID NO: 212 - DNA, 29 bp, Homo sapiens
  misc_feature (15)..(15): n is c or t
cttcctgaaagtccncaatgggaaagaaa

SEQ ID NO: 213 - DNA, 29 bp, Homo sapiens
  misc_feature (15)..(15): n is c or g
cagttcttcactctnatcaaaaatagact

SEQ ID NO: 214 - DNA, 29 bp, Homo sapiens
  misc_feature (15)..(15): n is c or t
acacatggaaaaganatgattgcatactg

SEQ ID NO: 215 - DNA, 29 bp, Homo sapiens
  misc_feature (15)..(15): n is g or t
atttatgaaatcatnattaattgtaagac

SEQ ID NO: 216 - DNA, 29 bp, Homo sapiens
  misc_feature (15)..(15): n is g or t
ttaattgttatcccnagtgtattccagag

SEQ ID NO: 217 - DNA, 29 bp, Homo sapiens
  misc_feature (15)..(15): n is a or c
cccagattcacattntataatatcaaaga

SEQ ID NO: 218 - DNA, 29 bp, Homo sapiens
  misc_feature (15)..(15): n is a or g
agcagctcagttacnatgtgtgaacggaa

SEQ ID NO: 219 - DNA, 29 bp, Homo sapiens
  misc_feature (15)..(15): n is c or t
actaagatattttanaaggtggcatgtag

SEQ ID NO: 220 - DNA, 29 bp, Homo sapiens
  misc_feature (15)..(15): n is c or t
ctggtggagtccttntaaaacattccatt

SEQ ID NO: 221 - DNA, 29 bp, Homo sapiens
  misc_feature (15)..(15): n is c or g
ttggtctgacatgcnctagtctgacagca

SEQ ID NO: 222 - DNA, 29 bp, Homo sapiens
  misc_feature (15)..(15): n is a or g
ttacttagccagatngtgccacacaggac

SEQ ID NO: 223 - DNA, 29 bp, Homo sapiens
  misc_feature (15)..(15): n is c or g
tatgtgagtgacatncatgcacaggttgg

SEQ ID NO: 224 - DNA, 29 bp, Homo sapiens
  misc_feature (15)..(15): n is c or t
aggcaaaacagactnttatgattcccatt

SEQ ID NO: 225 - DNA, 29 bp, Homo sapiens
  misc_feature (15)..(15): n is g or t
tatcttctggtgacntcacactagcagtg

SEQ ID NO: 226 - DNA, 29 bp, Homo sapiens
  misc_feature (15)..(15): n is a or g
tgtacggagatcgantcaccgtctattca

SEQ ID NO: 227 - DNA, 29 bp, Homo sapiens
  misc_feature (15)..(15): n is c or g
accaaggttagaccncattcaaaaatctg

SEQ ID NO: 228 - DNA, 29 bp, Homo sapiens
  misc_feature (15)..(15): n is a or g
ctgttaacaaattanagagttctacttaa

SEQ ID NO: 229 - DNA, 29 bp, Homo sapiens
  misc_feature (15)..(15): n is c or g
agaaggaagaaatgnagctctatgagagt

SEQ ID NO: 230 - DNA, 29 bp, Homo sapiens
  misc_feature (15)..(15): n is c or t
atagtggctattgcnattgagcatcagct

SEQ ID NO: 231 - DNA, 29 bp, Homo sapiens
  misc_feature (15)..(15): n is a or g
tgatctctttctctnctgcttagtttgac

SEQ ID NO: 232 - DNA, 29 bp, Homo sapiens
  misc_feature (15)..(15): n is a or g
tctccagggtgggcntgacaactaggtct

SEQ ID NO: 233 - DNA, 29 bp, Homo sapiens
  misc_feature (15)..(15): n is c or g
ccaggggtaagtccncaagaggttaatga

SEQ ID NO: 234 - DNA, 29 bp, Homo sapiens
  misc_feature (15)..(15): n is c or t
ctcacctcgagctgnccaagggcaccttc

SEQ ID NO: 235 - DNA, 29 bp, Homo sapiens
  misc_feature (15)..(15): n is c or t
ctcacctcgagctgnccaagggcaccttc

SEQ ID NO: 236 - DNA, 29 bp, Homo sapiens
  misc_feature (15)..(15): n is a or g
ccctggatgcaactntgcaccccagacag

SEQ ID NO: 237 - DNA, 29 bp, Homo sapiens
  misc_feature (15)..(15): n is a or g
ttgatagatgttgcntcacaacccgattt

SEQ ID NO: 238 - DNA, 29 bp, Homo sapiens
  misc_feature (15)..(15): n is c or t
gaaaaaacattccangcattcagggatga

SEQ ID NO: 239 - DNA, 29 bp, Homo sapiens
  misc_feature (15)..(15): n is c or t
gaaaaaacattccangcattcagggatga

SEQ ID NO: 240 - DNA, 29 bp, Homo sapiens
  misc_feature (15)..(15): n is c or t
tcctccatattacanatgttaatagaata

SEQ ID NO: 241 - DNA, 29 bp, Homo sapiens
  misc_feature (15)..(15): n is a or g
ctcaaaaaggatgcntgtcatgcactatt

SEQ ID NO: 242 - DNA, 29 bp, Homo sapiens
  misc_feature (15)..(15): n is c or t
ggaagtgcacgcttngaggtgggtcctgg

SEQ ID NO: 243 - DNA, 29 bp, Homo sapiens
  misc_feature (15)..(15): n is c or g
ccgcttgaaaatagncaaaaagtctactg

SEQ ID NO: 244 - DNA, 29 bp, Homo sapiens
  misc_feature (15)..(15): n is c or t
ttgttccatttttcnttactggaaggagt

SEQ ID NO: 245 - DNA, 29 bp, Homo sapiens
  misc_feature (15)..(15): n is c or g
ttgttattctcaaanaagatctttcacag

SEQ ID NO: 246 - DNA, 29 bp, Homo sapiens
  misc_feature (15)..(15): n is a or g
ggtgaaagagtgaangtagaaactgtggt

SEQ ID NO: 247 - DNA, 29 bp, Homo sapiens
  misc_feature (15)..(15): n is a or g
ggtgaaagagtgaangtagaaactgtggt

SEQ ID NO: 248 - DNA, 29 bp, Homo sapiens
  misc_feature (15)..(15): n is c or g
aatgtaatcataatnttacccaccttgat

SEQ ID NO: 249 - DNA, 29 bp, Homo sapiens
  misc_feature (15)..(15): n is a or g
tcctactctaatacnttcctcactactgt

SEQ ID NO: 250 - DNA, 29 bp, Homo sapiens
  misc_feature (15)..(15): n is c or t
gctatagtaaatganagaaaatgagtttg

SEQ ID NO: 251 - DNA, 29 bp, Homo sapiens
  misc_feature (15)..(15): n is a or t
ttgaaacatgggcanttttatctaagtaa

SEQ ID NO: 252 - DNA, 29 bp, Homo sapiens
  misc_feature (15)..(15): n is a or g
tctgttgttgcacanattcccaatgagat

SEQ ID NO: 253 - DNA, 29 bp, Homo sapiens
  misc_feature (15)..(15): n is c or t
tccagtactttacanctatgcctagttta

SEQ ID NO: 254 - DNA, 29 bp, Homo sapiens
  misc_feature (15)..(15): n is c or t
tggattattgatagncctatggcagacat

SEQ ID NO: 255 - DNA, 29 bp, Homo sapiens
  misc_feature (15)..(15): n is a or g
attgtcaaaggtgangtagccggtcaagg

SEQ ID NO: 256 - DNA, 29 bp, Homo sapiens
  misc_feature (15)..(15): n is a or g
tcaggagccagtaantccacacaggcagg

SEQ ID NO: 257 - DNA, 29 bp, Homo sapiens
  misc_feature (15)..(15): n is c or t
gcaaggcttggccantagcggatagatcc

SEQ ID NO: 258 - DNA, 29 bp, Homo sapiens
  misc_feature (15)..(15): n is a or t
gcttctcaaactgangaaggagcaattac

SEQ ID NO: 259 - DNA, 29 bp, Homo sapiens
  misc_feature (15)..(15): n is a or g
tttatatgtataaantcttctggaagcag

SEQ ID NO: 260 - DNA, 29 bp, Homo sapiens
  misc_feature (15)..(15): n is a or g
gtgaaagaactgacntgtagagagaataa

SEQ ID NO: 261 - DNA, 29 bp, Homo sapiens
  misc_feature (15)..(15): n is c or t
tcttgaacgagaaanggcgggactcttct

SEQ ID NO: 262 - DNA, 29 bp, Homo sapiens
  misc_feature (15)..(15): n is c or t
cccaactgcaaggcnttagggtgctgagc

SEQ ID NO: 263 - DNA, 29 bp, Homo sapiens
  misc_feature (15)..(15): n is c or t
agccatcgagatagnatcagggtattaat

SEQ ID NO: 264 - DNA, 29 bp, Homo sapiens
  misc_feature (15)..(15): n is c or g
gctcccggacttgtntattcaacccggtg

SEQ ID NO: 265 - DNA, 29 bp, Homo sapiens
  misc_feature (15)..(15): n is c or t
ggaatgaacagatanaattaatccaggtc

SEQ ID NO: 266 - DNA, 29 bp, Homo sapiens
  misc_feature (15)..(15): n is a or g
tgggggtggcggtangtaacaaaagatga

SEQ ID NO: 267 - DNA, 29 bp, Homo sapiens
  misc_feature (15)..(15): n is c or t
aaccagaacaatttnccagtgatattgag

SEQ ID NO: 268 - DNA, 29 bp, Homo sapiens
  misc_feature (15)..(15): n is a or g
atattgtaatttaanctgagtgattttct

SEQ ID NO: 269 - DNA, 29 bp, Homo sapiens
  misc_feature (15)..(15): n is a or g
tctttactatgtcanttaccaccacttac

SEQ ID NO: 270 - DNA, 29 bp, Homo sapiens
  misc_feature (15)..(15): n is c or t
ttatattgttttgtngttccttaaatgga

SEQ ID NO: 271 - DNA, 29 bp, Homo sapiens
  misc_feature (15)..(15): n is a or g
agtgcactcacttcntactagttaccctt

SEQ ID NO: 272 - DNA, 29 bp, Homo sapiens
  misc_feature (15)..(15): n is a or g
ggaaataaaaagganacagacataaagta

SEQ ID NO: 273 - DNA, 29 bp, Homo sapiens
  misc_feature (15)..(15): n is a or g
ggacaaacagtcccnattagcaaaattaa

SEQ ID NO: 274 - DNA, 29 bp, Homo sapiens
  misc_feature (15)..(15): n is a or g
gctaagccttaaacntgtttaaaaacaga

SEQ ID NO: 275 - DNA, 29 bp, Homo sapiens
  misc_feature (15)..(15): n is c or t
ccatgttatgatgtncggtcattgatttt

SEQ ID NO: 276 - DNA, 29 bp, Homo sapiens
  misc_feature (15)..(15): n is a or g
acactcacaaaatanttcatacactttcc

SEQ ID NO: 277 - DNA, 29 bp, Homo sapiens
  misc_feature (15)..(15): n is c or t
attagaacaaaatgnatagtaatataaca

SEQ ID NO: 278 - DNA, 29 bp, Homo sapiens
  misc_feature (15)..(15): n is a or g
tatactgtagtggtntacttgcgaataga

SEQ ID NO: 279 - DNA, 29 bp, Homo sapiens
  misc_feature (15)..(15): n is c or g
gtggaataactttcnctagttgacatgat

SEQ ID NO: 280 - DNA, 29 bp, Homo sapiens
  misc_feature (15)..(15): n is c or t
acactggcaaacagnacttcaatacaggg

SEQ ID NO: 281 - DNA, 29 bp, Homo sapiens
  misc_feature (15)..(15): n is c or g
gtgccattgggaaanaaaataatgtcatt

SEQ ID NO: 282 - DNA, 29 bp, Homo sapiens
  misc_feature (15)..(15): n is c or t
atttataaagttatngataaggatttatg

SEQ ID NO: 283 - DNA, 29 bp, Homo sapiens
  misc_feature (15)..(15): n is c or t
accaattggcacctnaagctctgcgccat

SEQ ID NO: 284 - DNA, 29 bp, Homo sapiens
  misc_feature (15)..(15): n is c or t
tactatgttcatatnttttttagcatttt

SEQ ID NO: 285 - DNA, 29 bp, Homo sapiens
  misc_feature (15)..(15): n is a or t
tcctcaccaaaaagntggatcactgtgaa

SEQ ID NO: 286 - DNA, 29 bp, Homo sapiens
  misc_feature (15)..(15): n is a or g
taataatgtccgtangtatagtagttacc

SEQ ID NO: 287 - DNA, 29 bp, Homo sapiens
  misc_feature (15)..(15): n is c or t
gaaaaccccttgacnagtacattatttag

SEQ ID NO: 288 - DNA, 29 bp, Homo sapiens
  misc_feature (15)..(15): n is c or t
ttggtagctgttganagtgattcatcaat

SEQ ID NO: 289 - DNA, 29 bp, Homo sapiens
  misc_feature (15)..(15): n is a or t
tactttgtcttgcanatgaggaatccgag

SEQ ID NO: 290 - DNA, 29 bp, Homo sapiens
  misc_feature (15)..(15): n is c or t
tatttctggtccatngcccttaaactgct

SEQ ID NO: 291 - DNA, 29 bp, Homo sapiens
  misc_feature (15)..(15): n is c or g
attaccaattctatnttgaggaatttctc

SEQ ID NO: 292 - DNA, 29 bp, Homo sapiens
  misc_feature (15)..(15): n is c or g
tactgagagaacatnatattattccccaa

SEQ ID NO: 293 - DNA, 29 bp, Homo sapiens
  misc_feature (15)..(15): n is c or t
aataagggaacagtntagagagactccag

SEQ ID NO: 294 - DNA, 29 bp, Homo sapiens
  misc_feature (15)..(15): n is a or g
caaacatttctaaantgagagctgtctaa

SEQ ID NO: 295 - DNA, 29 bp, Homo sapiens
  misc_feature (15)..(15): n is c or t
ccagagctggggaangccaccaagttgga

SEQ ID NO: 296 - DNA, 29 bp, Homo sapiens
  misc_feature (15)..(15): n is c or t
ccagagctggggaangccaccaagttgga

SEQ ID NO: 297 - DNA, 29 bp, Homo sapiens
  misc_feature (15)..(15): n is c or t
ccagagctggggaangccaccaagttgga

SEQ ID NO: 298 - DNA, 29 bp, Homo sapiens
  misc_feature (15)..(15): n is a or c
tttgatcatggttcncatcacacccatca

SEQ ID NO: 299 - DNA, 29 bp, Homo sapiens
  misc_feature (15)..(15): n is c or t
tcaatccaagagatncctcttctctctgg

SEQ ID NO: 300 - DNA, 29 bp, Homo sapiens
  misc_feature (15)..(15): n is c or t
ctacctacttcaganggttgtcatgagaa

SEQ ID NO: 301 - DNA, 29 bp, Homo sapiens
  misc_feature (15)..(15): n is g or t
aatagtacaatttgntggagaaggaggct

SEQ ID NO: 302 - DNA, 29 bp, Homo sapiens
  misc_feature (15)..(15): n is a or g
gtatctcaaaaattntcataaattatact

SEQ ID NO: 303 - DNA, 29 bp, Homo sapiens
  misc_feature (15)..(15): n is c or t
tgctcctctataaanatgtcgaataatta

SEQ ID NO: 304 - DNA, 29 bp, Homo sapiens
  misc_feature (15)..(15): n is c or t
actttacgatttcancatcctaagataac

SEQ ID NO: 305 - DNA, 29 bp, Homo sapiens
  misc_feature (15)..(15): n is c or g
tggaatgaattttanaaccagacatataa

SEQ ID NO: 306 - DNA, 29 bp, Homo sapiens
  misc_feature (15)..(15): n is a or g
atttgctgcatagtntatgggttatgttt

SEQ ID NO: 307 - DNA, 29 bp, Homo sapiens
  misc_feature (15)..(15): n is a or g
cagtgatttccactntatccttcaagcag

SEQ ID NO: 308 - DNA, 29 bp, Homo sapiens
  misc_feature (15)..(15): n is c or t
taatcttttagtaancaacacttatgtat

SEQ ID NO: 309 - DNA, 29 bp, Homo sapiens
  misc_feature (15)..(15): n is a or t
atttgccacattaantatgatgctgatct

SEQ ID NO: 310 - DNA, 29 bp, Homo sapiens
  misc_feature (15)..(15): n is a or t
ttttgatatcttttntaaaatacaaccaa

SEQ ID NO: 311 - DNA, 29 bp, Homo sapiens
  misc_feature (15)..(15): n is c or t
aatgttaactgttantattgttgccacct

SEQ ID NO: 312 - DNA, 29 bp, Homo sapiens
  misc_feature (15)..(15): n is a or c
tattcttccttctanaatgcatacacttt

SEQ ID NO: 313 - DNA, 29 bp, Homo sapiens
  misc_feature (15)..(15): n is c or t
cgtgccatatttggncttttcatgaaatg

SEQ ID NO: 314 - DNA, 29 bp, Homo sapiens
  misc_feature (15)..(15): n is a or g
agcaaggtctagaanctcatatcagcaag

SEQ ID NO: 315 - DNA, 29 bp, Homo sapiens
  misc_feature (15)..(15): n is c or t
tattctccttatcancagcagatcaaaac

SEQ ID NO: 316 - DNA, 29 bp, Homo sapiens
  misc_feature (15)..(15): n is a or g
attctgaaagagggntaagattttgccag

SEQ ID NO: 317 - DNA, 29 bp, Homo sapiens
  misc_feature (15)..(15): n is c or t
ccttattactaccanttttagaatgcctt

SEQ ID NO: 318 - DNA, 29 bp, Homo sapiens
  misc_feature (15)..(15): n is a or g
cctaaatcatcttanaagcagaaatctta

SEQ ID NO: 319 - DNA, 29 bp, Homo sapiens
  misc_feature (15)..(15): n is c or t
tatactatcaccaanaatttgttatcagc

SEQ ID NO: 320 - DNA, 29 bp, Homo sapiens
  misc_feature (15)..(15): n is c or t
agtgaacagtttccntattcccttctctt

SEQ ID NO: 321 - DNA, 29 bp, Homo sapiens
  misc_feature (15)..(15): n is a or g
acaccaaacagagtnctattttctttttg

SEQ ID NO: 322 - DNA, 29 bp, Homo sapiens
  misc_feature (15)..(15): n is a or t
cagatctaaaacagntaacactgaagaat

SEQ ID NO: 323 - DNA, 29 bp, Homo sapiens
  misc_feature (15)..(15): n is a or g
aacatactgtaattnttaactcttgccct

SEQ ID NO: 324 - DNA, 29 bp, Homo sapiens
  misc_feature (15)..(15): n is g or t
attcattcagacttncaagatatcattaa

SEQ ID NO: 325 - DNA, 29 bp, Homo sapiens
  misc_feature (15)..(15): n is c or t
gaatgattcccagantgagttctgcagca

SEQ ID NO: 326 - DNA, 29 bp, Homo sapiens
  misc_feature (15)..(15): n is c or t
tcccccttaagtttncaaaagtcctggaa

SEQ ID NO: 327 - DNA, 29 bp, Homo sapiens
  misc_feature (15)..(15): n is c or t
tcccccttaagtttncaaaagtcctggaa

SEQ ID NO: 328 - DNA, 29 bp, Homo sapiens
  misc_feature (15)..(15): n is g or t
agaattaaatcaaangaggattcggggac

SEQ ID NO: 329 - DNA, 29 bp, Homo sapiens
  misc_feature (15)..(15): n is a or t
taaagcctgctgatnttgaaaattgggtt

SEQ ID NO: 330 - DNA, 29 bp, Homo sapiens
  misc_feature (15)..(15): n is c or t
tctcaagtctaatanatgagccaaatgtt

SEQ ID NO: 331 - DNA, 29 bp, Homo sapiens
  misc_feature (15)..(15): n is c or t
ataccaaacccttanagaccaaatcatat

SEQ ID NO: 332 - DNA, 29 bp, Homo sapiens
  misc_feature (15)..(15): n is c or g
tgtaagttgagcttntagaattgcgagtt

SEQ ID NO: 333 - DNA, 29 bp, Homo sapiens
  misc_feature (15)..(15): n is a or t
tattacgaagcaagntatttcgagagtgg

SEQ ID NO: 334 - DNA, 29 bp, Homo sapiens
  misc_feature (15)..(15): n is a or g
aaaacttttgcgtanatatcatttctatg

SEQ ID NO: 335 - DNA, 29 bp, Homo sapiens
  misc_feature (15)..(15): n is a or c
tcatgtggaatatcnaaggtcatgcttat

SEQ ID NO: 336 - DNA, 29 bp, Homo sapiens
  misc_feature (15)..(15): n is c or g
tctagtaagacagangaagatacgatggg

SEQ ID NO: 337 - DNA, 29 bp, Homo sapiens
  misc_feature (15)..(15): n is a or g
gggtcaatcatgtcnttaatgtgtctggg

SEQ ID NO: 338 - DNA, 29 bp, Homo sapiens
  misc_feature (15)..(15): n is a or g
gtggcatatttgaanatgcaggtgaaaaa

SEQ ID NO: 339 - DNA, 29 bp, Homo sapiens
  misc_feature (15)..(15): n is a or c
taagaattttctatngtagattttaaact

SEQ ID NO: 340 - DNA, 29 bp, Homo sapiens
  misc_feature (15)..(15): n is a or c
gaacagagttagtanagcaaagaaggaat

SEQ ID NO: 341 - DNA, 29 bp, Homo sapiens
  misc_feature (15)..(15): n is g or t
tactcgtttttattncattaacttttctt

SEQ ID NO: 342 - DNA, 29 bp, Homo sapiens
  misc_feature (15)..(15): n is g or t
aatagtctacataanacgactttctctct

SEQ ID NO: 343 - DNA, 29 bp, Homo sapiens
  misc_feature (15)..(15): n is c or t
tttgcacattaacantgacctagtgcacc

SEQ ID NO: 344 - DNA, 29 bp, Homo sapiens
  misc_feature (15)..(15): n is a or g
tctggcaatcctacntggcaggtactaca

SEQ ID NO: 345 - DNA, 29 bp, Homo sapiens
  misc_feature (15)..(15): n is a or g
ctgtgggactctganaggttgagtctctg

SEQ ID NO: 346 - DNA, 29 bp, Homo sapiens
  misc_feature (15)..(15): n is a or g
tgcctagttagagcnctgtcaagttctct

SEQ ID NO: 347 - DNA, 29 bp, Homo sapiens
  misc_feature (15)..(15): n is a or g
aaaatagcagatganatatttgatcatgt

SEQ ID NO: 348 - DNA, 29 bp, Homo sapiens
  misc_feature (15)..(15): n is a or g
atattacaaagatanaaaagttaacgaac

SEQ ID NO: 349 - DNA, 29 bp, Homo sapiens
  misc_feature (15)..(15): n is c or g
aaaaacgattataanagtaagattagacc

SEQ ID NO: 350 - DNA, 29 bp, Homo sapiens
  misc_feature (15)..(15): n is a or t
ttctttttcgtttgntttagaagagaaaa

SEQ ID NO: 351 - DNA, 29 bp, Homo sapiens
  misc_feature (15)..(15): n is a or c
ttaaactgcacttcnagaaaagtttgtta

SEQ ID NO: 352 - DNA, 29 bp, Homo sapiens
  misc_feature (15)..(15): n is a or t
ctcagaatcaggacntataggtggactca

SEQ ID NO: 353 - DNA, 29 bp, Homo sapiens
  misc_feature (15)..(15): n is c or t
taagacttggcgtanattttaaataaata

SEQ ID NO: 354 - DNA, 29 bp, Homo sapiens
  misc_feature (15)..(15): n is a or g
ttcttttaaatggtntgaatattattctc

SEQ ID NO: 355 - DNA, 29 bp, Homo sapiens
  misc_feature (15)..(15): n is g or t
tttttctagtttatnactttaccattaaa

SEQ ID NO: 356 - DNA, 29 bp, Homo sapiens
  misc_feature (15)..(15): n is c or t
aatctctcttgcacntgccttgcagttta

SEQ ID NO: 357 - DNA, 29 bp, Homo sapiens
  misc_feature (15)..(15): n is c or t
atccctgagtggtancaagcaaccctggg

SEQ ID NO: 358 - DNA, 29 bp, Homo sapiens
  misc_feature (15)..(15): n is c or t
tacaatgtggtccangtatagatatagtt

SEQ ID NO: 359 - DNA, 29 bp, Homo sapiens
  misc_feature (15)..(15): n is c or g
aagacatcatgtacntcgtctggaagcaa

SEQ ID NO: 360 - DNA, 29 bp, Homo sapiens
  misc_feature (15)..(15): n is a or g
tctttcgtaagatangatcccccaatacc

SEQ ID NO: 361 - DNA, 29 bp, Homo sapiens
  misc_feature (15)..(15): n is a or g
tctttcgtaagatangatcccccaatacc

SEQ ID NO: 362 - DNA, 29 bp, Homo sapiens
  misc_feature (15)..(15): n is a or g
cacgatgaaagatcngaatgtggcacaag

SEQ ID NO: 363 - DNA, 29 bp, Homo sapiens
  misc_feature (15)..(15): n is c or g
taaaagcaccttcanaaaagcagatgaaa

SEQ ID NO: 364 - DNA, 29 bp, Homo sapiens
  misc_feature (15)..(15): n is g or t
taagttcagtttttngaactctccttaag

SEQ ID NO: 365 - DNA, 29 bp, Homo sapiens
  misc_feature (15)..(15): n is c or t
ctggtaggtgaatanggtgtttcattaca

SEQ ID NO: 366 - DNA, 29 bp, Homo sapiens
  misc_feature (15)..(15): n is a or c
ttactttaaaaaganatttgcctttatta

SEQ ID NO: 367 - DNA, 29 bp, Homo sapiens
  misc_feature (15)..(15): n is a or g
atgagaaatatttcngttacattaccatt

SEQ ID NO: 368 - DNA, 29 bp, Homo sapiens
  misc_feature (15)..(15): n is c or t
ggaattaaattcatntcaggtgctattgt

SEQ ID NO: 369 - DNA, 29 bp, Homo sapiens
  misc_feature (15)..(15): n is a or c
acttgttaaaaaaanctcatgaataggtc

SEQ ID NO: 370 - DNA, 29 bp, Homo sapiens
  misc_feature (15)..(15): n is c or t
tgcaaaaataccttntgttttcatagagc

SEQ ID NO: 371 - DNA, 29 bp, Homo sapiens
  misc_feature (15)..(15): n is c or t
cctgaaaggccaccngctataaacaagga

SEQ ID NO: 372 - DNA, 29 bp, Homo sapiens
  misc_feature (15)..(15): n is c or t
ttctatttccaatantgaagtttgccatt

SEQ ID NO: 373 - DNA, 29 bp, Homo sapiens
  misc_feature (15)..(15): n is g or t
tgcaagcaacttatncaacctcaccttta

SEQ ID NO: 374 - DNA, 29 bp, Homo sapiens
  misc_feature (15)..(15): n is c or t
ccaaggtctgacccntgggcctggcctgg

SEQ ID NO: 375 - DNA, 29 bp, Homo sapiens
  misc_feature (15)..(15): n is a or g
cctttatttatggangagtcattattttt

SEQ ID NO: 376 - DNA, 29 bp, Homo sapiens
  misc_feature (15)..(15): n is g or t
gattgctggctttantttcccattgtgta

SEQ ID NO: 377 - DNA, 29 bp, Homo sapiens
  misc_feature (15)..(15): n is c or t
tctgcttttgactanagccaaccttgtct

SEQ ID NO: 378 - DNA, 29 bp, Homo sapiens
  misc_feature (15)..(15): n is c or t
aagtgatgccttctntactccacctagtc

SEQ ID NO: 379 - DNA, 29 bp, Homo sapiens
  misc_feature (15)..(15): n is c or t
ctgagaaacgagacntggattacggaagt

SEQ ID NO: 380 - DNA, 29 bp, Homo sapiens
  misc_feature (15)..(15): n is a or g
aagaaacggtatacntaatggcactcagc

SEQ ID NO: 381 - DNA, 29 bp, Homo sapiens
  misc_feature (15)..(15): n is c or t
cccctaagatttcantggcaaaagtcagg

SEQ ID NO: 382 - DNA, 29 bp, Homo sapiens
  misc_feature (15)..(15): n is c or g
tttccaaagggaagntcatgggaaacgct

SEQ ID NO: 383 - DNA, 29 bp, Homo sapiens
  misc_feature (15)..(15): n is c or g
atccaaaatggtgantctgttcttgttga

SEQ ID NO: 384 - DNA, 29 bp, Homo sapiens
  misc_feature (15)..(15): n is c or t
ttctcctgagtaagnggagtcatgtgatt

SEQ ID NO: 385 - DNA, 29 bp, Homo sapiens
  misc_feature (15)..(15): n is a or g
acagaagcaaccctnaaatgctgtattgt

SEQ ID NO: 386 - DNA, 29 bp, Homo sapiens
  misc_feature (15)..(15): n is c or t
cttctgggacaggtnctgtgatgactgca

SEQ ID NO: 387 - DNA, 29 bp, Homo sapiens
  misc_feature (15)..(15): n is a or g
ctctggctgtttgancatgtgggaaatga

SEQ ID NO: 388 - DNA, 29 bp, Homo sapiens
  misc_feature (15)..(15): n is c or g
gggtctgtatgtgcntctggggaaaagca

SEQ ID NO: 389 - DNA, 29 bp, Homo sapiens
  misc_feature (15)..(15): n is c or t
agtcatgttcaactnatgttcccaaaggc

SEQ ID NO: 390 - DNA, 29 bp, Homo sapiens
  misc_feature (15)..(15): n is c or t
tggcttaatatggtnggaccaactcttca

SEQ ID NO: 391 - DNA, 29 bp, Homo sapiens
  misc_feature (15)..(15): n is c or t
tacagtggctactantgagattcagatat

SEQ ID NO: 392 - DNA, 29 bp, Homo sapiens
  misc_feature (15)..(15): n is a or c
tgttttcttgtctcntaagttttgaagta

SEQ ID NO: 393 - DNA, 29 bp, Homo sapiens
  misc_feature (15)..(15): n is c or t
gctagggttttaatntgacaatgaactgg

SEQ ID NO: 394 - DNA, 29 bp, Homo sapiens
  misc_feature (15)..(15): n is a or c
ctgtgtagatgagancccgaggagatggt

SEQ ID NO: 395 - DNA, 29 bp, Homo sapiens
  misc_feature (15)..(15): n is g or t
taaagattctaaacncctcttcattttat

SEQ ID NO: 396 - DNA, 29 bp, Homo sapiens
  misc_feature (15)..(15): n is g or t
taaagattctaaacncctcttcattttat

SEQ ID NO: 397 - DNA, 29 bp, Homo sapiens
  misc_feature (15)..(15): n is a or t
cagagttctccatgncagcttgtgtgtca

SEQ ID NO: 398 - DNA, 29 bp, Homo sapiens
  misc_feature (15)..(15): n is c or t
tttgtcaaactgaangacactaactttgg

SEQ ID NO: 399 - DNA, 29 bp, Homo sapiens
  misc_feature (15)..(15): n is c or t
gtggtaagaactaanttgatggcacaaga

SEQ ID NO: 400 - DNA, 29 bp, Homo sapiens
  misc_feature (15)..(15): n is c or g
taccttcttgctaantttcagattcagtt

SEQ ID NO: 401 - DNA, 29 bp, Homo sapiens
  misc_feature (15)..(15): n is a or g
atttgcaatgtggtnttatggccgtttgt

SEQ ID NO: 402 - DNA, 29 bp, Homo sapiens
  misc_feature (15)..(15): n is c or t
tccacatggtaaatnagctcagaattcaa

SEQ ID NO: 403 - DNA, 29 bp, Homo sapiens
  misc_feature (15)..(15): n is a or g
gactgaaataacatncccagtggacatta

SEQ ID NO: 404 - DNA, 29 bp, Homo sapiens
  misc_feature (15)..(15): n is a or t
atcatttcaattaancatgcacgaagtat

SEQ ID NO: 405 - DNA, 29 bp, Homo sapiens
  misc_feature (15)..(15): n is a or c
gtttcaggaaaagangagtagatacttcc

SEQ ID NO: 406 - DNA, 29 bp, Homo sapiens
  misc_feature (15)..(15): n is a or g
atctgaatgtcaaangatttcttctaatt

SEQ ID NO: 407 - DNA, 29 bp, Homo sapiens
  misc_feature (15)..(15): n is a or g
tgagctgcaacttgnaatttcctctgact

SEQ ID NO: 408 - DNA, 29 bp, Homo sapiens
  misc_feature (15)..(15): n is c or t
gtacaatattatcanataatcaatgtgcc

SEQ ID NO: 409 - DNA, 29 bp, Homo sapiens
  misc_feature (15)..(15): n is a or t
tagcctgaacatacnctatgtgggtggtc

SEQ ID NO: 410 - DNA, 29 bp, Homo sapiens
  misc_feature (15)..(15): n is c or g
cacactatcaaacanacaccgttcatagc

SEQ ID NO: 411 - DNA, 29 bp, Homo sapiens
  misc_feature (15)..(15): n is c or t
gggtacagcaacagnagccatttttatct

SEQ ID NO: 412 - DNA, 29 bp, Homo sapiens
  misc_feature (15)..(15): n is c or t
tgcacatttataaantgatatttgcaact

SEQ ID NO: 413 - DNA, 29 bp, Homo sapiens
  misc_feature (15)..(15): n is c or g
gaataaggctctacntttcagtctccctc

SEQ ID NO: 414 - DNA, 29 bp, Homo sapiens
  misc_feature (15)..(15): n is c or t
caggtatacaattanacatttttccccac

SEQ ID NO: 415 - DNA, 29 bp, Homo sapiens
  misc_feature (15)..(15): n is c or t
gttagaatactttangttgtctcctcttc

SEQ ID NO: 416 - DNA, 29 bp, Homo sapiens
  misc_feature (15)..(15): n is c or t
cttatcagtggctanaatgactatgagaa

SEQ ID NO: 417 - DNA, 29 bp, Homo sapiens
  misc_feature (15)..(15): n is c or g
attataaattttccnattaacaaattaat

SEQ ID NO: 418 - DNA, 29 bp, Homo sapiens
  misc_feature (15)..(15): n is g or t
ccaaactggaggtanttgaggcatcatgg

SEQ ID NO: 419 - DNA, 29 bp, Homo sapiens
  misc_feature (15)..(15): n is c or g
tcctggtgtgtcagnttgtccaagtgaaa

SEQ ID NO: 420 - DNA, 29 bp, Homo sapiens
  misc_feature (15)..(15): n is a or g
gcactcaaaaaattnattatggccgtcaa

SEQ ID NO: 421 - DNA, 29 bp, Homo sapiens
  misc_feature (15)..(15): n is c or g
atttgctgcttaganagatgataagaact

SEQ ID NO: 422 - DNA, 29 bp, Homo sapiens
  misc_feature (15)..(15): n is a or g
atgatggggtattanacttgtttctcaaa

SEQ ID NO: 423 - DNA, 29 bp, Homo sapiens
  misc_feature (15)..(15): n is a or t
ccttcagactcttanttattcatcattca

SEQ ID NO: 424 - DNA, 29 bp, Homo sapiens
  misc_feature (15)..(15): n is c or g
tgggtttccaaaganaggcaatttcctaa

SEQ ID NO: 425 - DNA, 29 bp, Homo sapiens
  misc_feature (15)..(15): n is g or t
ccatgttacaaaggnaggctacagagaag

SEQ ID NO: 426 - DNA, 29 bp, Homo sapiens
  misc_feature (15)..(15): n is a or g
tttattctaggtacntggagaactacatt

SEQ ID NO: 427 - DNA, 29 bp, Homo sapiens
  misc_feature (15)..(15): n is a or g
acaagataaagaaantcccactaccttca

SEQ ID NO: 428 - DNA, 29 bp, Homo sapiens
  misc_feature (15)..(15): n is a or g
ctgcctctcttctcnctatcgcattcatt

SEQ ID NO: 429 - DNA, 29 bp, Homo sapiens
  misc_feature (15)..(15): n is c or t
ttctcaccataaaanggagccaaaaacaa

SEQ ID NO: 430 - DNA, 29 bp, Homo sapiens
  misc_feature (15)..(15): n is a or g
tgatgccaagttcanatgtatttacatat

SEQ ID NO: 431 - DNA, 29 bp, Homo sapiens
  misc_feature (15)..(15): n is c or t
ttttttcccttagcngtcattttggatat

SEQ ID NO: 432 - DNA, 29 bp, Homo sapiens
  misc_feature (15)..(15): n is g or t
agcccaatcaatttnagagagacaagtgg

SEQ ID NO: 433 - DNA, 29 bp, Homo sapiens
  misc_feature (15)..(15): n is a or g
agaccatctagtctnatatgtctatataa

SEQ ID NO: 434 - DNA, 29 bp, Homo sapiens
  misc_feature (15)..(15): n is a or t
ctaagccttaaaaanatttcatcattaag

SEQ ID NO: 435 - DNA, 29 bp, Homo sapiens
  misc_feature (15)..(15): n is c or t
cccgccaaggctgangttccctaagcaac

SEQ ID NO: 436 - DNA, 29 bp, Homo sapiens
  misc_feature (15)..(15): n is a or g
aaatgagcaaaacgngttttagaaagtgt

SEQ ID NO: 437 - DNA, 29 bp, Homo sapiens
  misc_feature (15)..(15): n is a or t
atcactttgttccanctgagaagaaatgg

SEQ ID NO: 438 - DNA, 29 bp, Homo sapiens
  misc_feature (15)..(15): n is c or g
agctggctagctcantcattggagaaacc

SEQ ID NO: 439 - DNA, 29 bp, Homo sapiens
  misc_feature (15)..(15): n is a or c
aaacaccctaagggnattatttcgcgtag

SEQ ID NO: 440 - DNA, 29 bp, Homo sapiens
  misc_feature (15)..(15): n is a or g
tggcctatggttganaaattaattaaaca

SEQ ID NO: 441 - DNA, 29 bp, Homo sapiens
  misc_feature (15)..(15): n is a or g
ttatatttattcctntaacaagaaggaaa

SEQ ID NO: 442 - DNA, 29 bp, Homo sapiens
  misc_feature (15)..(15): n is a or g
cttataaagttcctntgggattttggtta

SEQ ID NO: 443 - DNA, 29 bp, Homo sapiens
  misc_feature (15)..(15): n is a or c
gtttccactacaacngctaccaccatatt

SEQ ID NO: 444 - DNA, 29 bp, Homo sapiens
  misc_feature (15)..(15): n is a or g
agagaaagcttttgnggaaaaaaagcatt

SEQ ID NO: 445 - DNA, 29 bp, Homo sapiens
  misc_feature (15)..(15): n is c or t
aaatgattccatctntttgcaaaaccgca

SEQ ID NO: 446 - DNA, 29 bp, Homo sapiens
  misc_feature (15)..(15): n is c or g
ttaattatttttaanaagtgctaatttgc

SEQ ID NO: 447 - DNA, 29 bp, Homo sapiens
  misc_feature (15)..(15): n is c or t
atcatgtgaaatgtngttattcatgtgaa

SEQ ID NO: 448 - DNA, 29 bp, Homo sapiens
  misc_feature (15)..(15): n is c or t
agtattcagaaaacnataatgattgaaga

SEQ ID NO: 449 - DNA, 29 bp, Homo sapiens
  misc_feature (15)..(15): n is c or t
agtattcagaaaacnataatgattgaaga

SEQ ID NO: 450 - DNA, 29 bp, Homo sapiens
  misc_feature (15)..(15): n is c or g
tcttgtgaattgcangtgtaggaggtgga

SEQ ID NO: 451 - DNA, 29 bp, Homo sapiens
  misc_feature (15)..(15): n is c or g
aagtgactgtgtcangctaagctcccttg

SEQ ID NO: 452 - DNA, 29 bp, Homo sapiens
  misc_feature (15)..(15): n is a or g
ttgattccaaaacangtgctttttaaatt

SEQ ID NO: 453 - DNA, 29 bp, Homo sapiens
  misc_feature (15)..(15): n is c or g
tcttttttgtcatanaagatacttcaaag

SEQ ID NO: 454 - DNA, 29 bp, Homo sapiens
  misc_feature (15)..(15): n is a or g
caaatgcaatattcnatttcctgggtcac

SEQ ID NO: 455 - DNA, 29 bp, Homo sapiens
  misc_feature (15)..(15): n is c or g
tgccagctttaactnactgacaaaataat

SEQ ID NO: 456 - DNA, 29 bp, Homo sapiens
  misc_feature (15)..(15): n is a or g
ttagactctcctccntgacctgagtcatt

SEQ ID NO: 457 - DNA, 29 bp, Homo sapiens
  misc_feature (15)..(15): n is c or t
ttattaagcaataangaaagacagtcttg

SEQ ID NO: 458 - DNA, 29 bp, Homo sapiens
  misc_feature (15)..(15): n is c or t
gacgcattcatacantgatatgcaaggaa

SEQ ID NO: 459 - DNA, 29 bp, Homo sapiens
  misc_feature (15)..(15): n is c or g
tacacttcattaagnacctgctatatacc

SEQ ID NO: 460 - DNA, 29 bp, Homo sapiens
  misc_feature (15)..(15): n is a or g
atgaattgcttagcncccctggccaaagt

SEQ ID NO: 461 - DNA, 29 bp, Homo sapiens
  misc_feature (15)..(15): n is c or t
gggaaaagtaaacangttagggactctgt

SEQ ID NO: 462 - DNA, 29 bp, Homo sapiens
  misc_feature (15)..(15): n is c or t
accatagctgcctanggaataagacccgt

SEQ ID NO: 463 - DNA, 29 bp, Homo sapiens
  misc_feature (15)..(15): n is a or c
ctatgtcagtagacnatgaaatcaaaaat

SEQ ID NO: 464 - DNA, 29 bp, Homo sapiens
  misc_feature (15)..(15): n is a or g
gctatagtaagtccntgtaacatttcaaa

SEQ ID NO: 465 - DNA, 29 bp, Homo sapiens
  misc_feature (15)..(15): n is c or t
aggggaaagaattgncaaatgtcacacag

SEQ ID NO: 466 - DNA, 29 bp, Homo sapiens
  misc_feature (15)..(15): n is c or t
ttctgctccaaatgnggtgtaaaatgaat

SEQ ID NO: 467 - DNA, 29 bp, Homo sapiens
  misc_feature (15)..(15): n is a or g
aataacctatcttancaacagtcatatct

SEQ ID NO: 468 - DNA, 29 bp, Homo sapiens
  misc_feature (15)..(15): n is a or g
ttagtgtgccaacangagagtaggagaat

SEQ ID NO: 469 - DNA, 29 bp, Homo sapiens
  misc_feature (15)..(15): n is c or t
agccattagaataanttccactgatatta

SEQ ID NO: 470 - DNA, 29 bp, Homo sapiens
  misc_feature (15)..(15): n is c or t
aaagtataaaatgangagtgttatatact

SEQ ID NO: 471 - DNA, 29 bp, Homo sapiens
  misc_feature (15)..(15): n is c or t
ttgatttctttgaanacaataggctttcc

SEQ ID NO: 472 - DNA, 29 bp, Homo sapiens
  misc_feature (15)..(15): n is a or g
ttttaagtgaacgtntattagactttaaa

SEQ ID NO: 473 - DNA, 29 bp, Homo sapiens
  misc_feature (15)..(15): n is a or t
ataaattttcatcancttgatatacatag

SEQ ID NO: 474 - DNA, 29 bp, Homo sapiens
  misc_feature (15)..(15): n is a or g
tgtggctggccacantcttggcaagcgct

SEQ ID NO: 475 - DNA, 29 bp, Homo sapiens
  misc_feature (15)..(15): n is a or g
acctgcacatgtgtngattctgtaatagg

SEQ ID NO: 476 - DNA, 29 bp, Homo sapiens
  misc_feature (15)..(15): n is a or c
agatggggcattganattaattgaaataa

SEQ ID NO: 477 - DNA, 29 bp, Homo sapiens
  misc_feature (15)..(15): n is a or g
atcccacttgtttcntggattcaatttgt

SEQ ID NO: 478 - DNA, 29 bp, Homo sapiens
  misc_feature (15)..(15): n is c or t
attcaaagcacatcntaaccacagaaaac

SEQ ID NO: 479 - DNA, 29 bp, Homo sapiens
  misc_feature (15)..(15): n is a or g
agtattcagatttcntcacgatatacagt

SEQ ID NO: 480 - DNA, 29 bp, Homo sapiens
  misc_feature (15)..(15): n is a or g
gaaaaagttgacccnctgtacttatttgt

SEQ ID NO: 481 - DNA, 29 bp, Homo sapiens
  misc_feature (15)..(15): n is a or g
ccacaatgttccacntcgcaggcctttgt

SEQ ID NO: 482 - DNA, 29 bp, Homo sapiens
  misc_feature (15)..(15): n is c or t
tcactacttcaaagntgaaatatcttatt

SEQ ID NO: 483 - DNA, 29 bp, Homo sapiens
  misc_feature (15)..(15): n is c or g
tcacttaggaatctnacattctggattct

SEQ ID NO: 484 - DNA, 29 bp, Homo sapiens
  misc_feature (15)..(15): n is a or g
tttcctttacctcantgaatcatctacat

SEQ ID NO: 485 - DNA, 29 bp, Homo sapiens
  misc_feature (15)..(15): n is c or t
cagtatcagttttancttgagctcctttt

SEQ ID NO: 486 - DNA, 29 bp, Homo sapiens
  misc_feature (15)..(15): n is a or g
caaatacttgctctntaaaatcagtttta

SEQ ID NO: 487 - DNA, 29 bp, Homo sapiens
  misc_feature (15)..(15): n is c or t
tacggaagttatgangattataggcacca

SEQ ID NO: 488 - DNA, 29 bp, Homo sapiens
  misc_feature (15)..(15): n is a or g
aggaagatacatccnttgaacccaacagg

SEQ ID NO: 489 - DNA, 29 bp, Homo sapiens
  misc_feature (15)..(15): n is a or c
atttggaatcctcangtagagggtttggg

SEQ ID NO: 490 - DNA, 29 bp, Homo sapiens
  misc_feature (15)..(15): n is c or g
acattctctgtcatnctgcaaagtgtcta

SEQ ID NO: 491 - DNA, 29 bp, Homo sapiens
  misc_feature (15)..(15): n is c or t
ccactcaaaggaatnagaagttcagagaa

SEQ ID NO: 492 - DNA, 29 bp, Homo sapiens
  misc_feature (15)..(15): n is a or g
ttcactgtgatgcanctgcacttcctgac

SEQ ID NO: 493 - DNA, 29 bp, Homo sapiens
  misc_feature (15)..(15): n is a or g
gacagtaggttggtngtatgaaattaaca

SEQ ID NO: 494 - DNA, 29 bp, Homo sapiens
  misc_feature (15)..(15): n is a or g
aaatctgtgctactnttacaatctggacc

SEQ ID NO: 495 - DNA, 29 bp, Homo sapiens
  misc_feature (15)..(15): n is c or t
cactcacatacctangtatttccaaatgg

SEQ ID NO: 496 - DNA, 29 bp, Homo sapiens
  misc_feature (15)..(15): n is c or t
agaaaggctgtattncataaaggaactgg

SEQ ID NO: 497 - DNA, 29 bp, Homo sapiens
  misc_feature (15)..(15): n is c or t
agtcagagattaatntacaaatgtcgcct

SEQ ID NO: 498 - DNA, 29 bp, Homo sapiens
  misc_feature (15)..(15): n is g or t
ataaagtatttatantccaggcatttcat

SEQ ID NO: 499 - DNA, 29 bp, Homo sapiens
  misc_feature (15)..(15): n is a or c
tcacatctcaaatcnatcacattttattt

SEQ ID NO: 500 - DNA, 29 bp, Homo sapiens
  misc_feature (15)..(15): n is c or g
agattaatgataaanggatctggaaaaga

SEQ ID NO: 501 - DNA, 29 bp, Homo sapiens
  misc_feature (15)..(15): n is a or g
attgtgaaggttacntgtatcatgttcaa

SEQ ID NO: 502 - DNA, 29 bp, Homo sapiens
  misc_feature (15)..(15): n is a or g
atgatatcttagagntattgataagctgg

SEQ ID NO: 503 - DNA, 29 bp, Homo sapiens
  misc_feature (15)..(15): n is c or g
ctttgtatacatccngctgcaaagaaacg

SEQ ID NO: 504 - DNA, 29 bp, Homo sapiens
  misc_feature (15)..(15): n is a or g
catagaaatgttctngattcattccttat

SEQ ID NO: 505 - DNA, 29 bp, Homo sapiens
  misc_feature (15)..(15): n is c or t
gcaaaaatccatctnggttaagagttcag

SEQ ID NO: 506 - DNA, 29 bp, Homo sapiens
  misc_feature (15)..(15): n is c or t
aagcactctggatcnttccatactgcaat

SEQ ID NO: 507 - DNA, 29 bp, Homo sapiens
  misc_feature (15)..(15): n is a or g
acattctgtgaaagnaaacagtactagct

SEQ ID NO: 508 - DNA, 29 bp, Homo sapiens
  misc_feature (15)..(15): n is a or c
cctgttggaacaagngcaagctacgatag

SEQ ID NO: 509 - DNA, 29 bp, Homo sapiens
  misc_feature (15)..(15): n is a or c
cctgttggaacaagngcaagctacgatag

SEQ ID NO: 510 - DNA, 29 bp, Homo sapiens
  misc_feature (15)..(15): n is a or g
tgtgagcctcctatncattaaaagccaga

SEQ ID NO: 511 - DNA, 29 bp, Homo sapiens
  misc_feature (15)..(15): n is g or t
ttcttgttacctaanaaaggagtcagtca

SEQ ID NO: 512 - DNA, 29 bp, Homo sapiens
  misc_feature (15)..(15): n is a or g
cattttttttctctnttttagactgcaag

SEQ ID NO: 513 - DNA, 29 bp, Homo sapiens
  misc_feature (15)..(15): n is c or t
tttcttaaaacatgnacccctgagcccac

SEQ ID NO: 514 - DNA, 29 bp, Homo sapiens
  misc_feature (15)..(15): n is c or t
tgctttagttaaccnagttggcttttgtt

SEQ ID NO: 515 - DNA, 29 bp, Homo sapiens
  misc_feature (15)..(15): n is a or g
ggatttggagggacnaagtagaaagttct

SEQ ID NO: 516 - DNA, 29 bp, Homo sapiens
  misc_feature (15)..(15): n is a or g
tgaattttgtgcacngcagatgccatatt

SEQ ID NO: 517 - DNA, 29 bp, Homo sapiens
  misc_feature (15)..(15): n is a or g
ctgagtagctcatcntcatcccaaagact

SEQ ID NO: 518 - DNA, 29 bp, Homo sapiens
  misc_feature (15)..(15): n is c or t
ctctaatgggtgaangtaaatttggggca

SEQ ID NO: 519 - DNA, 29 bp, Homo sapiens
  misc_feature (15)..(15): n is a or c
tgaaaagtgcagtanaatagttttttaaa

SEQ ID NO: 520 - DNA, 29 bp, Homo sapiens
  misc_feature (15)..(15): n is a or g
ccttaaaagtaaacntctctcgtgatctc

SEQ ID NO: 521 - DNA, 29 bp, Homo sapiens
  misc_feature (15)..(15): n is c or t
gtgtcagtaagccangtgatcaatggagg

SEQ ID NO: 522 - DNA, 29 bp, Homo sapiens
  misc_feature (15)..(15): n is a or t
aagagaaatcaggtntgagaaacactggg

SEQ ID NO: 523 - DNA, 29 bp, Homo sapiens
  misc_feature (15)..(15): n is c or t
ggtagtgttacatangaatccatttttat

SEQ ID NO: 524 - DNA, 29 bp, Homo sapiens
  misc_feature (15)..(15): n is a or g
cgggctttctctggnttagcctcactaac

SEQ ID NO: 525 - DNA, 29 bp, Homo sapiens
  misc_feature (15)..(15): n is c or t
gtttgtaaatttaantcattcgatatgtc

SEQ ID NO: 526 - DNA, 29 bp, Homo sapiens
  misc_feature (15)..(15): n is a or t
tgtcactattggaangtggtatgactgta

SEQ ID NO: 527 - DNA, 29 bp, Homo sapiens
  misc_feature (15)..(15): n is a or g
aaccatgatttgaangctctccctacaca

SEQ ID NO: 528 - DNA, 29 bp, Homo sapiens
  misc_feature (15)..(15): n is c or t
gcaacatttccaggntgtacttgaggtta

SEQ ID NO: 529 - DNA, 29 bp, Homo sapiens
  misc_feature (15)..(15): n is a or g
ctcaggctctgagcngacgtggcttaggt

SEQ ID NO: 530 - DNA, 29 bp, Homo sapiens
  misc_feature (15)..(15): n is c or t
tggtctgaaagagcngaaggtacaacaga

SEQ ID NO: 531 - DNA, 29 bp, Homo sapiens
  misc_feature (15)..(15): n is a or g
acactcttttctgcntgtatttaagctaa

SEQ ID NO: 532 - DNA, 29 bp, Homo sapiens
  misc_feature (15)..(15): n is a or g
acactcttttctgcntgtatttaagctaa

SEQ ID NO: 533 - DNA, 29 bp, Homo sapiens
  misc_feature (15)..(15): n is a or g
acactcttttctgcntgtatttaagctaa

SEQ ID NO: 534 - DNA, 29 bp, Homo sapiens
  misc_feature (15)..(15): n is g or t
ttgtagttctcagancactttctcattca

SEQ ID NO: 535 - DNA, 29 bp, Homo sapiens
  misc_feature (15)..(15): n is g or t
ttgtagttctcagancactttctcattca

SEQ ID NO: 536 - DNA, 29 bp, Homo sapiens
  misc_feature (15)..(15): n is g or t
ttgtagttctcagancactttctcattca

SEQ ID NO: 537 - DNA, 29 bp, Homo sapiens
  misc_feature (15)..(15): n is c or t
taaaactggagatanggtttttaacactg

SEQ ID NO: 538 - DNA, 29 bp, Homo sapiens
  misc_feature (15)..(15): n is c or t
taaaactggagatanggtttttaacactg

SEQ ID NO: 539 - DNA, 29 bp, Homo sapiens
  misc_feature (15)..(15): n is c or t
aaagccttcttggantgggtgtcttcgct

SEQ ID NO: 540 - DNA, 29 bp, Homo sapiens
  misc_feature (15)..(15): n is c or t
gatttttcactaaangaggaatagggaga

SEQ ID NO: 541 - DNA, 29 bp, Homo sapiens
  misc_feature (15)..(15): n is c or t
cttgattatagtatnaacacactatcaca

SEQ ID NO: 542 - DNA, 29 bp, Homo sapiens
  misc_feature (15)..(15): n is a or g
agatacatagatacnggaaaacattttag

SEQ ID NO: 543 - DNA, 29 bp, Homo sapiens
  misc_feature (15)..(15): n is c or t
ccagtccaacccagngttttttcagacca

SEQ ID NO: 544 - DNA, 29 bp, Homo sapiens
  misc_feature (15)..(15): n is c or t
ccagtccaacccagngttttttcagacca

SEQ ID NO: 545 - DNA, 29 bp, Homo sapiens
  misc_feature (15)..(15): n is a or g
aagaaaagaaccctntgcaaagacactga

SEQ ID NO: 546 - DNA, 29 bp, Homo sapiens
  misc_feature (15)..(15): n is c or t
cccgacattctgctntctaccctatggtt

SEQ ID NO: 547 - DNA, 29 bp, Homo sapiens
  misc_feature (15)..(15): n is a or c
gtgtaaacagtttgnggtacagtacactc

SEQ ID NO: 548 - DNA, 29 bp, Homo sapiens
  misc_feature (15)..(15): n is a or g
gaaagaggaaatacngactcagtaactct

SEQ ID NO: 549 - DNA, 29 bp, Homo sapiens
  misc_feature (15)..(15): n is c or t
gtgagatggtcagcncaatttcccaaaca

SEQ ID NO: 550 - DNA, 29 bp, Homo sapiens
  misc_feature (15)..(15): n is t or g
tgctttctggcttcngcctgatgttatcg

SEQ ID NO: 551 - DNA, 29 bp, Homo sapiens
  misc_feature (15)..(15): n is a or g
ggcactgataccaantaaatctggatatc

SEQ ID NO: 552 - DNA, 29 bp, Homo sapiens
  misc_feature (15)..(15): n is a or g
agcatgcagaggganctctaaaaatactg

SEQ ID NO: 553 - DNA, 29 bp, Homo sapiens
  misc_feature (15)..(15): n is a or g
actcctgttaagcantcatctggcatttg

SEQ ID NO: 554 - DNA, 29 bp, Homo sapiens
  misc_feature (15)..(15): n is c or t
tgctagctgcttgangattattatctagt

SEQ ID NO: 555 - DNA, 29 bp, Homo sapiens
  misc_feature (15)..(15): n is c or t
ggcaaattatctgantttatcttctgata

SEQ ID NO: 556 - DNA, 29 bp, Homo sapiens
  misc_feature (15)..(15): n is c or t
aagaatgagttttancagttttaaatgga

SEQ ID NO: 557 - DNA, 29 bp, Homo sapiens
  misc_feature (15)..(15): n is g or t
aaattgctaacaccntatttgctgggagg

SEQ ID NO: 558 - DNA, 29 bp, Homo sapiens
  misc_feature (15)..(15): n is c or g
atattccagtctagngtggaaaattgtga

SEQ ID NO: 559 - DNA, 29 bp, Homo sapiens
  misc_feature (15)..(15): n is a or g
aatgagagattcatntctatgtcagttag

SEQ ID NO: 560 - DNA, 29 bp, Homo sapiens
  misc_feature (15)..(15): n is c or t
cagtaactagggaanttctgagccaaaag

SEQ ID NO: 561 - DNA, 29 bp, Homo sapiens
  misc_feature (15)..(15): n is c or t
tgcaccaccttccangtcatgactatatt

SEQ ID NO: 562 - DNA, 29 bp, Homo sapiens
  misc_feature (15)..(15): n is a or g
aatggcacttaactnatctttccctgctt

SEQ ID NO: 563 - DNA, 29 bp, Homo sapiens
  misc_feature (15)..(15): n is g or t
acagaaggtaataanttctgctccatgga

SEQ ID NO: 564 - DNA, 29 bp, Homo sapiens
  misc_feature (15)..(15): n is a or g
caattaataaggagncccacaggaacagt

SEQ ID NO: 565 - DNA, 29 bp, Homo sapiens
  misc_feature (15)..(15): n is g or t
gtggatggaagcttntaaagatgaatcat

SEQ ID NO: 566 - DNA, 29 bp, Homo sapiens
  misc_feature (15)..(15): n is a or g
caagtacaaaaacanctttcaaagaagag

SEQ ID NO: 567 - DNA, 29 bp, Homo sapiens
  misc_feature (15)..(15): n is a or g
ttacgagaagtacangttgtgagagaatt

SEQ ID NO: 568 - DNA, 29 bp, Homo sapiens
  misc_feature (15)..(15): n is c or t
ctcattatatccaanatttgctgtctagt

SEQ ID NO: 569 - DNA, 29 bp, Homo sapiens
  misc_feature (15)..(15): n is a or g
tcacactgatagaanagggaagatactgt

SEQ ID NO: 570 - DNA, 29 bp, Homo sapiens
  misc_feature (15)..(15): n is a or t
atgggagactttttnaaagaggaatgatg

SEQ ID NO: 571 - DNA, 29 bp, Homo sapiens
  misc_feature (15)..(15): n is c or t
aaatatagcaataancaggttgcatgttg

SEQ ID NO: 572 - DNA, 29 bp, Homo sapiens
  misc_feature (15)..(15): n is a or g
cttgcagactccacntgtcccctttcact

SEQ ID NO: 573 - DNA, 29 bp, Homo sapiens
  misc_feature (15)..(15): n is a or g
gccagacctagtatngagcagtcacataa

SEQ ID NO: 574 - DNA, 29 bp, Homo sapiens
  misc_feature (15)..(15): n is a or t
tgatccctaccatanccttgtggaaagca

SEQ ID NO: 575 - DNA, 29 bp, Homo sapiens
  misc_feature (15)..(15): n is a or g
aacccttaaattctntgacttcacagttg

SEQ ID NO: 576 - DNA, 29 bp, Homo sapiens
  misc_feature (15)..(15): n is c or t
attctaagtaagcangtgttagggtgtga

SEQ ID NO: 577 - DNA, 29 bp, Homo sapiens
  misc_feature (15)..(15): n is c or t
tgggtgataacctanattgacaaagggct

SEQ ID NO: 578 - DNA, 29 bp, Homo sapiens
  misc_feature (15)..(15): n is c or g
ctcactaaatgcctngcatggaattaagt

SEQ ID NO: 579 - DNA, 29 bp, Homo sapiens
  misc_feature (15)..(15): n is g or t
tctctcaagcacatncccttggtaagaaa

SEQ ID NO: 580 - DNA, 29 bp, Homo sapiens
  misc_feature (15)..(15): n is c or t
agcacagttcccagntatgcttcctgtgc

SEQ ID NO: 581 - DNA, 29 bp, Homo sapiens
  misc_feature (15)..(15): n is c or t
taaaaaacaagtatnacaatcacaagtat

SEQ ID NO: 582 - DNA, 29 bp, Homo sapiens
  misc_feature (15)..(15): n is c or t
aactcagggcattcngagcaaataggaat

SEQ ID NO: 583 - DNA, 29 bp, Homo sapiens
  misc_feature (15)..(15): n is a or g
tttataagctgctgnaccattcagaagtt

SEQ ID NO: 584 - DNA, 29 bp, Homo sapiens
  misc_feature (15)..(15): n is a or g
ggacttactgaaaangcaaattggccagg

SEQ ID NO: 585 - DNA, 29 bp, Homo sapiens
  misc_feature (15)..(15): n is c or t
ctctttttctgggangggacaggctaaaa

SEQ ID NO: 586 - DNA, 29 bp, Homo sapiens
  misc_feature (15)..(15): n is a or g
tccttataacgaganctgctctcattact

SEQ ID NO: 587 - DNA, 29 bp, Homo sapiens
  misc_feature (15)..(15): n is c or t
aaagttataaaaagntccatatattaaat

SEQ ID NO: 588 - DNA, 29 bp, Homo sapiens
  misc_feature (15)..(15): n is g or t
ttgctactaaatgtnactaaaatgttact

SEQ ID NO: 589 - DNA, 29 bp, Homo sapiens
  misc_feature (15)..(15): n is a or c
gaaactcctagtaanctccgaagggcaaa

SEQ ID NO: 590 - DNA, 29 bp, Homo sapiens
  misc_feature (15)..(15): n is c or t
gggagagcggctccncagttgtttgtttc

SEQ ID NO: 591 - DNA, 29 bp, Homo sapiens
  misc_feature (15)..(15): n is c or g
aaatgggagcgtganattaaaagaagatt

SEQ ID NO: 592 - DNA, 29 bp, Homo sapiens
  misc_feature (15)..(15): n is g or t
tactggtcctttatnaatcttaggcatga

SEQ ID NO: 593 - DNA, 29 bp, Homo sapiens
  misc_feature (15)..(15): n is c or g
ttctctgaactccanaataaccacttctt

SEQ ID NO: 594 - DNA, 29 bp, Homo sapiens
  misc_feature (15)..(15): n is c or g
agaggaaagagccgngcatggtaggaaaa

SEQ ID NO: 595 - DNA, 29 bp, Homo sapiens
  misc_feature (15)..(15): n is a or g
cactgagaaataagngctttaagagcagt

SEQ ID NO: 596 - DNA, 29 bp, Homo sapiens
  misc_feature (15)..(15): n is c or t
tacctgagctaaatnatagctgcacattc

SEQ ID NO: 597 - DNA, 29 bp, Homo sapiens
  misc_feature (15)..(15): n is c or t
ttgggctgtccaggnggaagtcaccagag

SEQ ID NO: 598 - DNA, 29 bp, Homo sapiens
  misc_feature (15)..(15): n is c or t
cttgtagggtggaangccagagggtgctc

SEQ ID NO: 599 - DNA, 29 bp, Homo sapiens
  misc_feature (15)..(15): n is a or t
cttacatttaaggtnatctttcaatatgg

SEQ ID NO: 600 - DNA, 29 bp, Homo sapiens
  misc_feature (15)..(15): n is a or g
gggacaagaagtttnttcctaacccacct

SEQ ID NO: 601 - DNA, 29 bp, Homo sapiens
  misc_feature (15)..(15): n is c or t
taagcaaaacatacngcaggttccagtgg

SEQ ID NO: 602 - DNA, 29 bp, Homo sapiens
  misc_feature (15)..(15): n is c or t
ctagaaatgggtaangtgtattgaatgta

SEQ ID NO: 603 - DNA, 29 bp, Homo sapiens
  misc_feature (15)..(15): n is c or t
tgctcatctctggcncggggtctcgtgat

SEQ ID NO: 604 - DNA, 29 bp, Homo sapiens
  misc_feature (15)..(15): n is c or g
gattctatcctctgnttggaagacacatt

SEQ ID NO: 605 - DNA, 29 bp, Homo sapiens
  misc_feature (15)..(15): n is c or g
tgcacacaggagtcnagctttgtttttgt

SEQ ID NO: 606 - DNA, 29 bp, Homo sapiens
  misc_feature (15)..(15): n is a or g
gctcattaagcgccngtgttctgagggga

SEQ ID NO: 607 - DNA, 29 bp, Homo sapiens
  misc_feature (15)..(15): n is a or g
tactcaaaggactangtggttaattctga

SEQ ID NO: 608 - DNA, 29 bp, Homo sapiens
  misc_feature (15)..(15): n is c or t
ggaccaagatgacanaggtttcctggtgg

SEQ ID NO: 609 - DNA, 29 bp, Homo sapiens
  misc_feature (15)..(15): n is a or c
cataaacaattaaanatttttcaaactca

SEQ ID NO: 610 - DNA, 29 bp, Homo sapiens
  misc_feature (15)..(15): n is a or c
tactgtacattttcntgttttcaaggtgt

SEQ ID NO: 611 - DNA, 29 bp, Homo sapiens
  misc_feature (15)..(15): n is c or t
atatttggtacaacntaagttcagtaaaa

SEQ ID NO: 612 - DNA, 29 bp, Homo sapiens
  misc_feature (15)..(15): n is c or t
gtcactattctagcngaagtctgtccacg

SEQ ID NO: 613 - DNA, 29 bp, Homo sapiens
  misc_feature (15)..(15): n is a or c
gtgactttcttacanagttgcttataact

SEQ ID NO: 614 - DNA, 29 bp, Homo sapiens
  misc_feature (15)..(15): n is c or t
cttaaagcctattcnatctgagaggcaat

SEQ ID NO: 615 - DNA, 29 bp, Homo sapiens
  misc_feature (15)..(15): n is c or t
atgtcatccacacanatggtttcaggtct

SEQ ID NO: 616 - DNA, 29 bp, Homo sapiens
  misc_feature (15)..(15): n is c or t
ataaatttgtgttangaatttttgtgttc

SEQ ID NO: 617 - DNA, 29 bp, Homo sapiens
  misc_feature (15)..(15): n is c or g
acctgctataggagnagctgttgatccag

SEQ ID NO: 618 - DNA, 29 bp, Homo sapiens
  misc_feature (15)..(15): n is a or c
atggggatataaccnaggagtgtccaagg

SEQ ID NO: 619 - DNA, 29 bp, Homo sapiens
  misc_feature (15)..(15): n is c or t
aggaatgtaagaganagccatctagtggt

SEQ ID NO: 620 - DNA, 29 bp, Homo sapiens
  misc_feature (15)..(15): n is c or g
aaattgaagtgtacnaaatgcttgatagg

SEQ ID NO: 621 - DNA, 29 bp, Homo sapiens
  misc_feature (15)..(15): n is a or g
gtttgtacattctancatctgcagggttt

SEQ ID NO: 622 - DNA, 29 bp, Homo sapiens
  misc_feature (15)..(15): n is a or c
caaagaggctggctnagaacgttgaagca

SEQ ID NO: 623 - DNA, 29 bp, Homo sapiens
  misc_feature (15)..(15): n is a or c
caaagaggctggctnagaacgttgaagca

SEQ ID NO: 624 - DNA, 29 bp, Homo sapiens
  misc_feature (15)..(15): n is g or t
agggacacagtcacnttccacagtaacta

SEQ ID NO: 625 - DNA, 29 bp, Homo sapiens
  misc_feature (15)..(15): n is g or t
agggacacagtcacnttccacagtaacta

SEQ ID NO: 626 - DNA, 29 bp, Homo sapiens
  misc_feature (15)..(15): n is c or t
ctaaaatgcaaatgncctttgaaggccag

SEQ ID NO: 627 - DNA, 29 bp, Homo sapiens
  misc_feature (15)..(15): n is c or t
aatcagatttagctncagaaataggaatg

SEQ ID NO: 628 - DNA, 29 bp, Homo sapiens
  misc_feature (15)..(15): n is c or g
tcagaagttcagcanatagcagctatata

SEQ ID NO: 629 - DNA, 29 bp, Homo sapiens
  misc_feature (15)..(15): n is a or g
gctctccatctcttnttcctgatccttcc

SEQ ID NO: 630 - DNA, 29 bp, Homo sapiens
  misc_feature (15)..(15): n is a or g
tgcttaattttctancagagcgttttctc

SEQ ID NO: 631 - DNA, 29 bp, Homo sapiens
  misc_feature (15)..(15): n is a or g
ctctaaagacaaagnttcagcattaccac

SEQ ID NO: 632 - DNA, 29 bp, Homo sapiens
  misc_feature (15)..(15): n is a or g
tacaacttataattntgctttgcctggca

SEQ ID NO: 633 - DNA, 29 bp, Homo sapiens
  misc_feature (15)..(15): n is c or g
tagcaccaaacaagngaaaatgtgttcca

SEQ ID NO: 634 - DNA, 29 bp, Homo sapiens
  misc_feature (15)..(15): n is c or t
tcttgcctatgtagnttctccacaaatac

SEQ ID NO: 635 - DNA, 29 bp, Homo sapiens
  misc_feature (15)..(15): n is a or g
agctggttttttaantaaaatgagacttt

SEQ ID NO: 636 - DNA, 29 bp, Homo sapiens
  misc_feature (15)..(15): n is a or c
aaactccacaaatcnaaagtatgtcatgt

SEQ ID NO: 637 - DNA, 29 bp, Homo sapiens
  misc_feature (15)..(15): n is g or t
agcaaactctgagangatcaagctaagat

SEQ ID NO: 638 - DNA, 29 bp, Homo sapiens
  misc_feature (15)..(15): n is g or t
gcagatgtgtttgcntgtactcagtacta

SEQ ID NO: 639 - DNA, 29 bp, Homo sapiens
  misc_feature (15)..(15): n is c or t
gtcctgtgcacctcnccgagccgtgatag

SEQ ID NO: 640 - DNA, 29 bp, Homo sapiens
  misc_feature (15)..(15): n is a or g
agaaggagctcaccntgtggacagggtac

SEQ ID NO: 641 - DNA, 29 bp, Homo sapiens
  misc_feature (15)..(15): n is a or g
gccaggcgggaccgngactcagggacaag

SEQ ID NO: 642 - DNA, 29 bp, Homo sapiens
  misc_feature (15)..(15): n is c or g
tttcaataataaatncaacttttaaaaat

SEQ ID NO: 643 - DNA, 29 bp, Homo sapiens
  misc_feature (15)..(15): n is c or t
tctctgagcctgaanttcctcagtgcatg

SEQ ID NO: 644 - DNA, 29 bp, Homo sapiens
  misc_feature (15)..(15): n is a or g
caagaaaagttagangtctctcatttagt

SEQ ID NO: 645 - DNA, 29 bp, Homo sapiens
  misc_feature (15)..(15): n is a or g
taatgtttctacagntctggtgtcttccc

SEQ ID NO: 646 - DNA, 29 bp, Homo sapiens
  misc_feature (15)..(15): n is c or t
aacgcagccgaccantcttccacaggctg

SEQ ID NO: 647 - DNA, 29 bp, Homo sapiens
  misc_feature (15)..(15): n is c or t
aattatacttgtctnttaaggatcaattc

SEQ ID NO: 648 - DNA, 29 bp, Homo sapiens
  misc_feature (15)..(15): n is g or t
ttgattttatctttntaaatctctaatta

SEQ ID NO: 649 - DNA, 29 bp, Homo sapiens
  misc_feature (15)..(15): n is c or t
atttattgtcaacanagaataatcttcca

SEQ ID NO: 650 - DNA, 29 bp, Homo sapiens
  misc_feature (15)..(15): n is a or g
ctttctactgcatgncaaatgttaatttg

SEQ ID NO: 651 - DNA, 29 bp, Homo sapiens
  misc_feature (15)..(15): n is a or g
tttctattcctcacnttgccttaaataca

SEQ ID NO: 652 - DNA, 29 bp, Homo sapiens
  misc_feature (15)..(15): n is a or g
agtggtgaaacatcntagattaaagtttt

SEQ ID NO: 653 - DNA, 29 bp, Homo sapiens
  misc_feature (15)..(15): n is c or g
aattagatgaacaanatgcaagcatagaa

SEQ ID NO: 654 - DNA, 29 bp, Homo sapiens
  misc_feature (15)..(15): n is c or t
cacacactaaagtgntgcacagaaacaaa

SEQ ID NO: 655 - DNA, 29 bp, Homo sapiens
  misc_feature (15)..(15): n is c or t
tcttgttttttatangaaaattcttcacc

SEQ ID NO: 656 - DNA, 29 bp, Homo sapiens
  misc_feature (15)..(15): n is c or t
cgattcctgtggggnttttcttcctcccc

SEQ ID NO: 657 - DNA, 29 bp, Homo sapiens
  misc_feature (15)..(15): n is a or t
cctagagtcacaggncaatagaaagagta

SEQ ID NO: 658 - DNA, 29 bp, Homo sapiens
  misc_feature (15)..(15): n is c or t
gatttaaatgaaaangttaggacatatga

SEQ ID NO: 659 - DNA, 29 bp, Homo sapiens
  misc_feature (15)..(15): n is a or g
tctatatcataattnaatgaacagatcag

SEQ ID NO: 660 - DNA, 29 bp, Homo sapiens
  misc_feature (15)..(15): n is a or g
ctctgcctttgggangaatggccctttct

SEQ ID NO: 661 - DNA, 29 bp, Homo sapiens
  misc_feature (15)..(15): n is a or g
ctctgcctttgggangaatggccctttct

SEQ ID NO: 662 - DNA, 29 bp, Homo sapiens
  misc_feature (15)..(15): n is a or g
tattctgtcttaaantacagtttgtgggc

SEQ ID NO: 663 - DNA, 29 bp, Homo sapiens
  misc_feature (15)..(15): n is c or t
gttccgtctgctagnttgtaattaaaggt

SEQ ID NO: 664 - DNA, 29 bp, Homo sapiens
  misc_feature (15)..(15): n is c or t
gtaatgtgcttccanagaaagaggccgta

SEQ ID NO: 665 - DNA, 29 bp, Homo sapiens
  misc_feature (15)..(15): n is a or g
tcaggatttcaaatntgttgatctttatt

SEQ ID NO: 666 - DNA, 29 bp, Homo sapiens
  misc_feature (15)..(15): n is g or t
tagaagcactcaatntctatttttagcta

SEQ ID NO: 667 - DNA, 29 bp, Homo sapiens
  misc_feature (15)..(15): n is c or t
tctcatttaagattnggacaatatcattg

SEQ ID NO: 668 - DNA, 29 bp, Homo sapiens
  misc_feature (15)..(15): n is c or t
tcccagggaggagtnctctgattcatggg

SEQ ID NO: 669 - DNA, 29 bp, Homo sapiens
  misc_feature (15)..(15): n is c or t
catatggctggaacncaaagttatttttt

SEQ ID NO: 670 - DNA, 29 bp, Homo sapiens
  misc_feature (15)..(15): n is c or t
tgaattgaatatcantgaagtttatgaat

SEQ ID NO: 671 - DNA, 29 bp, Homo sapiens
  misc_feature (15)..(15): n is c or t
tataaacttctttantggtcccagatctt

SEQ ID NO: 672 - DNA, 29 bp, Homo sapiens
  misc_feature (15)..(15): n is a or t
acctgaggaagccanatagttggtcaccc

SEQ ID NO: 673 - DNA, 29 bp, Homo sapiens
  misc_feature (15)..(15): n is c or g
tgattaatttccaanaagaaaaggtgccg

SEQ ID NO: 674 - DNA, 29 bp, Homo sapiens
  misc_feature (15)..(15): n is c or t
gtttattttaacttntatatgactgttat

SEQ ID NO: 675 - DNA, 29 bp, Homo sapiens
  misc_feature (15)..(15): n is c or t
tggagaaagaaatcnggatcagcagctcg

SEQ ID NO: 676 - DNA, 29 bp, Homo sapiens
  misc_feature (15)..(15): n is g or t
cagagagagctggtnctattgttaatggg

SEQ ID NO: 677 - DNA, 29 bp, Homo sapiens
  misc_feature (15)..(15): n is c or g
tgctgtctgattgantttgcaaaacgacc

SEQ ID NO: 678 - DNA, 29 bp, Homo sapiens
  misc_feature (15)..(15): n is a or g
aaattagtaaacaangccaaagatgtttt

SEQ ID NO: 679 - DNA, 29 bp, Homo sapiens
  misc_feature (15)..(15): n is c or t
taaaaaaggaagacntctgtgaacctaaa

SEQ ID NO: 680 - DNA, 29 bp, Homo sapiens
  misc_feature (15)..(15): n is a or g
aggccaaggacaggntccacctgaagcca

SEQ ID NO: 681 - DNA, 29 bp, Homo sapiens
  misc_feature (15)..(15): n is a or g
tcaaaaataaagttncccttgaactaatt

SEQ ID NO: 682 - DNA, 29 bp, Homo sapiens
  misc_feature (15)..(15): n is a or g
aacccaatgtgatcnttctccatgttccc

SEQ ID NO: 683 - DNA, 29 bp, Homo sapiens
  misc_feature (15)..(15): n is a or g
gctggagcagagctntaatcttgtgtaat

SEQ ID NO: 684 - DNA, 29 bp, Homo sapiens
  misc_feature (15)..(15): n is c or g
gaataggctcttctncttggacagacagc

SEQ ID NO: 685 - DNA, 29 bp, Homo sapiens
  misc_feature (15)..(15): n is a or g
aacgtgtagagctcnttgaagtcttctaa

SEQ ID NO: 686 - DNA, 29 bp, Homo sapiens
  misc_feature (15)..(15): n is c or t
aattagctgaactanggaattgcagattg

SEQ ID NO: 687 - DNA, 29 bp, Homo sapiens
  misc_feature (15)..(15): n is a or g
gtaagctatgttttnagatttccataaat

SEQ ID NO: 688 - DNA, 29 bp, Homo sapiens
  misc_feature (15)..(15): n is a or t
cctttgaggatacanagccttttctggtt

SEQ ID NO: 689 - DNA, 29 bp, Homo sapiens
  misc_feature (15)..(15): n is a or g
actgcttaaaggtgnacttattgtaaaat

SEQ ID NO: 690 - DNA, 29 bp, Homo sapiens
  misc_feature (15)..(15): n is a or c
taactttaggtaagngctttactcatgaa

SEQ ID NO: 691 - DNA, 29 bp, Homo sapiens
  misc_feature (15)..(15): n is c or t
agccctagcctttanggttttgccaggat

SEQ ID NO: 692 - DNA, 29 bp, Homo sapiens
  misc_feature (15)..(15): n is c or g
acattatttcttctntagaagctcctttc

SEQ ID NO: 693 - DNA, 29 bp, Homo sapiens
  misc_feature (15)..(15): n is c or t
catcgtactcggtcncgtgtgaaaagcag

SEQ ID NO: 694 - DNA, 29 bp, Homo sapiens
  misc_feature (15)..(15): n is c or t
tccatctctggctanaggagcccatgatg

SEQ ID NO: 695 - DNA, 29 bp, Homo sapiens
  misc_feature (15)..(15): n is a or g
aggtgccttaaattnactaattgaagact

SEQ ID NO: 696 - DNA, 29 bp, Homo sapiens
  misc_feature (15)..(15): n is a or g
cagtgggctcccaangactgtgagaagat

SEQ ID NO: 697 - DNA, 29 bp, Homo sapiens
  misc_feature (15)..(15): n is a or g
gctaaatatgcacantaccaggcctcctc

SEQ ID NO: 698 - DNA, 29 bp, Homo sapiens
  misc_feature (15)..(15): n is c or g
tgaagagtatatctntgaagaaaagggag

SEQ ID NO: 699 - DNA, 29 bp, Homo sapiens
  misc_feature (15)..(15): n is c or t
taaaataaaggctcnggattgttctcttg

SEQ ID NO: 700 - DNA, 29 bp, Homo sapiens
  misc_feature (15)..(15): n is c or t
ggtcaatgccactangcagcatctctgtt

SEQ ID NO: 701 - DNA, 29 bp, Homo sapiens
  misc_feature (15)..(15): n is c or t
ttttcatgccactanatttctcagtgttt

SEQ ID NO: 702 - DNA, 29 bp, Homo sapiens
  misc_feature (15)..(15): n is c or g
gaattcggctaaatncaagagaaagagat

SEQ ID NO: 703 - DNA, 29 bp, Homo sapiens
  misc_feature (15)..(15): n is c or t
tgacagcagacaagnatgccttccaaaaa

(19) United States
(12) Patent Application Publication
(10) Pub. No.: US 2006/0177847 A1
(43) Pub. Date: Aug. 10, 2006

(54) Markers for metabolic syndrome obesity and insulin resistance

(75) Inventors: David R. Cox, Belmont, CA (US); Dennis Ballinger, Menlo Park, CA (US); Richard Deane Hockett JR., Fishers, IN (US); Sandra Close Kirkwood, Fishers, IN (US)

Correspondence Address: QUINE INTELLECTUAL PROPERTY LAW GROUP, P.C., P O BOX 458, ALAMEDA, CA, 94501, US

(73) Assignee: Perlegen Sciences, Inc., Mountain View, CA (US)

(21) Appl. No.: 11/299,298

(22) Filed: Dec. 8, 2005

Related U.S. Application Data
(60) Provisional application No. 60/635,281, filed on Dec. 9, 2004.
(60) Provisional application No. 60/643,006, filed on Jan. 11, 2005.
(60) Provisional application No. 60/711,802, filed on Aug. 25, 2005.

(51) Int. Cl. C12Q 1/68

(52) U.S. Cl. 435/6

(57) ABSTRACT

Correlations between polymorphisms and metabolic syndrome, obesity, treatment-emergent weight gain and insulin resistance are provided. Methods of diagnosing and treating metabolic syndrome, obesity, treatment-emergent weight gain and insulin resistance are provided. Systems and kits for disgnosis and treatment of metabolic syndrome, treatment-emergent weight gain, obesity and insulin resistance are provided.

### CROSS REFERENCE TO RELATED APPLICATIONS

This application claims priority to and benefit of: U.S. Ser. No. 60/635,281 “Markers For Metabolic Syndrome Obesity And Insulin Resistance” by Cox and Ballinger, filed Dec. 9, 2004; U.S. Ser. No. 60/643,006 “Markers For Metabolic Syndrome Obesity And Insulin Resistance” by Cox and Ballinger, filed Jan. 11, 2005; and U.S. Ser. No. 60/711,802 “Markers For Metabolic Syndrome Obesity And Insulin Resistance” by Cox and Ballinger, filed Aug. 25, 2005, each of which is incorporated in its entirety for all purposes.

### BACKGROUND OF THE INVENTION

Metabolic syndrome is a collection of health disorders or risks that increase the chance of developing heart disease, stroke, and diabetes. The condition is also known by other names, including Syndrome X, insulin resistance syndrome, and dysmetabolic syndrome. Metabolic syndrome can include any of a variety of underlying metabolic phenotypes, including insulin resistance and/or obesity predisposition phenotypes.

Metabolic syndrome is often characterized by any of a number of metabolic disorders or risk factors, which are generally considered to most typify metabolic syndrome when more than one of these factors are present in a single individual. The factors include: central obesity (disproportionate fat tissue in and around the abdomen), atherogenic dyslipidemia (these include a family of blood fat disorders including, e.g., high triglycerides and low HDL cholesterol, that can foster plaque buildups in the vascular system, including artery walls), high blood pressure (130/85 mmHg or higher), insulin resistance or glucose intolerance (the inability to properly use insulin or blood sugar), a chronic prothrombotic state (e.g., characterized by high fibrinogen or plasminogen activator inhibitor [−1] levels in the blood), and a chronic proinflammatory state (e.g., characterized by higher than normal levels of high-sensitivity C-reactive protein in the blood). People with metabolic syndrome are at increased risk of coronary heart disease, other diseases related to plaque buildups in artery walls (e.g., stroke and peripheral vascular disease) and Type 2 Diabetes.

Furthermore, predisposition to obesity, metabolic syndrome, insulin resistance and/or the like can occur in patient populations exposed to any of a variety of environmental factors. For example, obesity predisposition can manifest itself as a simple predisposition to put on weight when exposed to a modern diet, or it can arise as a result of specific triggering events. One factor that can lead to obesity is termed “treatment-emergent weight gain,” a significant weight problem that arises for patients undergoing any of a variety of therapeutic treatment regimines. For example, treatment-emergent weight gain observed during antipsychotic therapy (e.g., treatment using atypical antipsychotic medications, e.g., olanzapine) is a significant clinical concern and, it is likely that genetic factors play a significant role in treatment-emergent weight gain, just as they do for obesity, metabolic syndrome and insulin resistance. Indeed, the genetic contribution to weight gain for treatment emergent weight gain has been investigated using a candidate gene approach (reviewed, e.g., by Muller et al. (2004) “Pharmacogenetics of antipsychotic-induced weight gain” Pharmacol. Res. 49:309-329). Although significant associations with candidate genes such as the Serotonin 5-HT_{2c} Receptor Gene (Reynolds et al. (2002) “Association of antipsychotic drug-induced weight gain with 5-HT2c receptor gene polymorphism” Lancet 359:2086-2087) and CYP2D6 (Ellingrod et al. (2002) “CYP2D6 polymorphisms and atypical antipsychotic weight gain.” Psychiatr. Genet. 12:55-58) have been reported, negative results have also been described (Muller et al. (2004) “Pharmacogenetics of antipsychotic-induced weight gain” Pharmacol. Res. 2004;49:309-329, Hong et al. “Genetic variants of the serotonin system and weight change during clozapine treatment” Pharmacogenetics 11:265-268). The lack of consistent findings has led to uncertainty as to the significance of several reported associations.

Metabolic syndrome is extremely common, particularly in the United States, where roughly 50 million people are thought to have the disorder. Roughly one in five Americans has metabolic syndrome. The number of people with metabolic syndrome increases with age, affecting more than 40 percent of people in their 60s and 70s. The underlying causes of Metabolic syndrome are, in many respects, quite unclear—though certain effects of the disorder such as obesity and lack of physical activity are often causal in nature as well. Given inheritance patterns for the disorder, there also appear to be genetic factors that underlie the syndrome.

For example, some people with metabolic syndrome are genetically predisposed to insulin resistance, which typically leads to obesity. On the other hand, obesity can and does also elicit insulin resistance. Thus, while it is true that most people with insulin resistance have central obesity, it is not always clear whether insulin resistance causes central obesity or whether central obesity causes insulin resistance. The underlying biological mechanism(s) between insulin resistance and metabolic risk factors (at the molecular level) are not fully understood and are also likely to be quite complex.

Not only is metabolic syndrome likely a result of several interacting genetic and environmental factors, but the criteria for diagnosing metabolic syndrome are somewhat variable. Criteria considered most relevant by the “Third Report of the National Cholesterol Education Program (NCEP) Expert Panel on Detection, Evaluation, and Treatment of High Blood Cholesterol in Adults (Adult Treatment Panel III)” in the diagnosis of metabolic disorder provide one widely used current set of diagnostic criteria.

Under the NCEP criteria, metabolic syndrome can be clinically identified by presence of three or more of the following components in a single patient: (1) central obesity, as measured by waist circumference (women with a waist circumference greater than 35 inches; for men greater than 40 inches); (2) fasting blood triglycerides greater than or equal to 150 mg/dL; (3) blood HDL cholesterol (for women less than 50 mg/dL, for men less than 40 mg/dL); (4) blood pressure greater than or equal to 130/85 mmHg; and (5) fasting glucose greater than or equal to 110 mg/dL. Other features such as insulin resistance (e.g., increased fasting blood insulin), prothrombotic state or proinflammatory state are not generally required for clinical diagnosis, though they are certainly also indicative of metabolic syndrome and follow-up studies on these attributes can be used to further confirm diagnosis of metabolic syndrome. For example, insulin resistance, even in the absence of the NCEP criteria, is often indicative of metabolic syndrome.

Treatment for metabolic syndrome, obesity, treatment emergent weight gain, insulin resistance, etc., can include a variety of clinical approaches, including weight loss and exercise (these two safest and most effective treatments are also often quite difficult to achieve in practice), and dietary changes. These dietary changes include: maintaining a diet that limits carbohydrates to 50 percent or less of total calories; eating foods defined as complex carbohydrates, such as whole grain bread (instead of white), brown rice (instead of white), sugars that are unrefined, increasing fiber consumption by eating legumes (for example, beans), whole grains, fruits and vegetables, reducing intake of red meats and poultry, consumption of “healthy” fats, such as those in olive oil, flaxseed oil and nuts, limiting alcohol intake, etc. In addition, treatment of blood pressure, and blood triglyceride levels can be controlled by a variety of available drugs (e.g., cholesterol modulating drugs), as can clotting disorders (e.g., via aspirin therapy) and in general, prothrombotic or proinflammatory states. If metabolic syndrome leads to diabetes, there are, of course, many treatments available for this disease, including those noted above, in conjunction with insulin treatment.

Thus, while there are a variety of treatments for treatment emergent weight gain, metabolic syndrome, obesity predisposition, insulin resistance etc., such as diet and exercise, drug therapy, etc., the molecular basis for these disorders is not clear, making diagnosis of these metabolic disorders problematic and the design of therapeutic agents to treat them quite difficult.

This is not to say, however, that certain progress has not been made towards understanding the molecular basis for, e.g., metabolic syndrome. It is clear, for example, that the brain monitors energy needs by assessing blood glucose and neural signals from the periphery. The mechanisms for glucose sensing and energy homeostasis in the brain are reviewed by Levin et al. (1999) “Brain glucose sensing and body energy homeostasis: role in obesity and diabetes” Am J. Physiol. 276 (regulatory Integrative Comp. Physiol.) R1223-R1231. The brain has a variety of neurons that directly sense glucose levels, as well as being able to receive neural inputs from glucosensors in the periphery. For example, glucose-responsive (GR) neurons increase and glucose sensitive (GS) neurons decrease their firing rate when brain glucose levels rise. GR neurons use an ATP-sensitive K⁺ channel to regulate neuronal firing rate, while the mechanism for GS neurons is unclear. Both diabetes and obesity (key causes or effects of metabolic syndrome) are associated with alterations in brain glucose sensing. GR neurons are hyporesponsive to glucose in animals with diet induced obesity and hyperinsulinemia. Insulin-dependent diabetic rats have been shown to have abnormalities in GR neurons and neurotransmitter systems involved with brain glucose sensing. However, the role of brain glucose sensing in the physiological regulation of energy balance in the pathophysiology of obesity and diabetes is not clear.

At least one report (Maekawa et al. (2000) “Localization of Glucokinase-Like Immunoreactivity in the Rat Lower Brain Stem: for Possible Location of Brain Glucose0Sensing Mechanisms,” Endrocrinology 141(1): 375-384) suggests that the location of glucose sensing apparatus in the brain includes ependymocytes, endothelial cells and many serotonergic neurons. In this study, an immunohistochemical approach was used to identify brain cells and sub-cellular locations that were immunoreactive with antibodies to pancreatic glucokinase (“GK”), which is a prerequisite enzyme in the glucose sensing apparatus of pancreatic β-cells. Cells that were immunoreactive with GK antibodies were further analyzed for the presence of glucose transporter-like immunoreactivities by immunohistochemically probing GK positive cells with antibodies to various glucotransporters (GLUT-1, GLUT-2, GLUT-4).

An understanding of which cells and subcellular structures were found to be implicated by this immunohistochemical analysis to likely be involved in glucose sensing is useful in considering possible mechanisms and structures of action for the glucose sensing apparatus in the brain. Maekawa (2000), above, showed that GK-positive ependymocytes were found to have glucose transporter-2(GLUT2)-like immunoreactivities on the cilia. In addition, the ependymocytes had GLUT1-like immunoreactivity on cilia and GLUT4-like immunoreactivity densely in cytoplasmic areas of the cells, as well as in plasma membranes in the cells. In serotonergic neurons, GK-like immunoreactivity was also found in the cytoplasm and their processes.

The presence of glucose sensors on the cilia of ependymocytes is interesting, e.g., because it is possible that ciliated ependymocytes detect alterations in cerebrospinal fluid (CSF) directly. Because glucose passes from the blood to the CSF to establish a concentration gradient between the two, the brain could monitor blood glucose by monitoring CSF glucose levels. Ciliated structures in general have been implicated in a very wide variety of sensing and signal transduction processes, pattern formation processes, cerebrosplinal and other fluid flow processes, mucociliary clearance, renal pathology, etc. Ciliary functions are reviewed, e.g., in Tallon et al. (2003) “To beat or not to beat: roles of cilia in development and disease” 12(1) R27-R35. Other relevant references relating to cilia function include: Eberl et al. (2000) “Genetically Similar Transduction Mechanisms for Touch and Hearing in Drosophila The Journal of Neuroscience 20(16):5981-5988 (role of cilia in touch and hearing); Zhang et al. (2002) “A sperm-associated WD Repeat Protein Orthologous to Chlamydomonas PF20 Associates with Spag6, the Mammalian Orthologue of Chlamydomonas PF16” Molecular and Cellular Biology 22(22): 7993-8004 (role of cilia in sperm motility); Pennarun et al. (2002) “Isolation and Expression of the Human hPF20 Gene Orthologous to Chlamydomonas pf20: Evaluation as a Candidate for Axonemal Defects of respiratory Cilia and Sperm Flagella” Am. J. Respir. Cell Biolog. 26:362-370 (role of cilia in primary ciliary dyskinesia, e.g., associated with situs inversus, Kartagner's Syndrome and male infertility); Bartoloni et al. (2002) “Mutations in the DNAH11 (axonemal heavy chain dynein type 11) gene cause one form of situs inversus totatlis and most likely primary ciliary dyskinesia” PNAS 99(16):10282-10286 (role of cilia in primary ciliary dyskinesia, e.g., associated with situs inversus, Kartagner's Syndrome and male infertility); and Supp et al. (1997) “Mutation of an axonemal dynein affects left-right asymmetry in inversus viscerum mice” Nature 389:963-966 (role of ciliary protein in inversus viscerum). Further, the correlation between certain diseases that involve ciliary proteins, such as polycystic kidney disease (PKD) and the risk of developing diabetes mellitus have been at least preliminarily observed (Duclox et al. (1999) “Polycystic kidney disease as a risk factor for post-transplant diabetes mellitus” Nephrol Dial Transplant 14:1244-1246.

Thus, while a considerable amount is known about metabolic syndrome, obesity, insulin resistance, and even treatment emergent weight gain, at the clinical level, disease diagnosis for these central human diseases is relatively imprecise, and early detection of susceptible individuals is difficult. Further, while various brain structures have been implicated in glucose sensing, no correlation between these structures and metabolic disorders such as metabolic syndrome has previously been shown. The present invention provides a number of new genetic correlations between metabolic syndrome (including e.g., obesity predisposition and insulin resistance), treatment emergent weight gain, etc., and various polymorphic alleles, providing the basis for improved diagnosis of disease, early detection of susceptible individuals (e.g., before metabolic syndrome or weight gain is clinically manifested), targets for potential disease modulators, as well as an improved understanding of metabolic syndrome, obesity, and treatment emergent weight gain at the molecular and cellular level. These and other features of the invention will be apparent upon review of the following.

### SUMMARY OF THE INVENTION

This invention provides previously unknown correlations between various polymorphisms and treatment emergent weight gain, metabolic syndrome, obesity predisposition and/or insulin resistance. The detection of these polymorphisms, accordingly, provides robust and precise methods and systems for identifying patients that have or are at risk for metabolic syndrome, obesity predisposition and/or insulin resistance. In addition, the identification of these polymorphisms provides high-throughput systems and methods for identifying modulators of treatment emergent weight gain, metabolic syndrome, obesity predisposition and/or insulin resistance.

Accordingly, in a first aspect, methods of identifying a treatment emergent weight gain phenotype, a metabolic syndrome phenotype, an insulin resistance phenotype, or an obesity predisposition phenotype for an organism or biological sample derived therefrom are provided. The method includes detecting, in the organism or biological sample, a polymorphism of a gene or at a locus closely linked thereto. Example genes encode a protein such as pregnancy associated plasma protein A (PAPPA), peptidylglycine alpha amidating monooxygenase (PAM), pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, and/or HSF2, in which the polymorphism is associated with the metabolic syndrome phenotype, an insulin resistance phenotype, or an obesity predisposition phenotype. Similarly, detecting a polymorphism of Appendix 1, or a locus closely linked thereto, can be used to identify a polymorphism associated with the treatment emergent weight gain phenotype, metabolic syndrome phenotype, insulin resistance phenotype, or obesity predisposition phenotype. In either case, presence of the relevant polymorphism is correlated to the treatment emergent weight gain phenotype, metabolic syndrome phenotype, the insulin resistance phenotype, or the obesity predisposition phenotype, thereby identifying the relevant phenotype.

Any of the features of metabolic syndrome can constitute the relevant phenotype, e.g., the phenotype can include insulin resistance, central obesity, atherogenic dyslipidemia, high blood pressure, glucose intolerance, a chronic prothrombotic state, a chronic proinflammatory state, etc. Thus, treatment emergent weight gain phenotype, obesity predisposition and insulin resistance phenotypes overlap with metabolic syndrome, along with the markers used herein to detect them.

The organism or the biological sample can be, or can be derived from, a mammal. For example, the organism can be a human patient, or the biological sample can be derived from a human patient (blood, lymph, skin, tissue, saliva, primary or secondary cell cultures derived therefrom, etc.).

Detecting the polymorphism can include amplifying the polymorphism or a sequence associated therewith and detecting the resulting amplicon. For example, amplifying the polymorphism can include admixing an amplification primer or amplification primer pair with a nucleic acid template isolated from the organism or biological sample. The primer or primer pair is typically complementary or partially complementary to at least a portion of the gene or other polymorphism, or to a proximal sequence thereto, and is capable of initiating nucleic acid polymerization by a polymerase on the nucleic acid template. The amplification can also include extending the primer or primer pair in a DNA polymerization reaction using a polymerase and the template nucleic acid to generate the amplicon. The amplicon can be detected by hybridizing the amplicon to an array, digesting the amplicon with a restriction enzyme, real-time PCR analysis, sequencing of the amplicon, or the like. Optionally, amplification can include performing a polymerase chain reaction (PCR), reverse transcriptase PCR (RT-PCR), or ligase chain reaction (LCR) using nucleic acid isolated from the organism or biological sample as a template in the PCR, RT-PCR, or LCR. Other formats can include allele specific hybridization, single nucleotide extension, or the like.

The polymorphism can be any detectable polymorphism, e.g., a SNP. For example, the allele can be any of those noted in Appendix 1. The alleles can positively correlate to treatment emergent weight gain, metabolic syndrome, obesity predisposition and/or insulin resistance, or can correlate negatively. Examples of each are described in Appendix 1.

Polymorphisms closely linked to PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, and/or HSF2, and/or any polymorphism of Appendix 1 can be used as markers for metabolic syndrome, obesity predisposition and/or insulin resistance. Such closely linked markers are typically about 20 cM or less, e.g., 15 cM or less, often 10 cM or less and, in certain preferred embodiments, 5 cM or less from the gene or other polymorphism of interest (e.g., an allelic marker locus in Appendix 1). The linked markers can, of course be closer than 5 cM, e.g., 4, 3, 2, 1, 0.5, 0.25, 0.1 cM or less from the gene or marker locus of Appendix 1. In general, the closer the linkage (or association), the more predictive the linked marker is of an allele of the gene or given marker locus (or association).

In one typical embodiment, correlating the polymorphism is performed by referencing a look up table that comprises correlations between alleles of the polymorphism and the phenotype. This table can be, e.g., a paper or electronic database comprising relevant correlation information. In one aspect, the database can be a multidimensional database comprising multiple correlations and taking multiple correlation relationships into account, simultaneously. Accessing the look up table can include extracting correlation information through a table look-up or can include more complex statistical analysis, such as principle component analysis (PCA), heuristic algorithms that track and/or update correlation information (e.g., neural networks), hidden Markov modeling, or the like.

Correlation information is useful for determining disease susceptibility (e.g., patient susceptibility to obesity, insulin resistance and/or metabolic syndrome), disease diagnosis (e.g., diagnosis of metabolic syndrome), and disease prognosis (e.g., likelihood that conventional therapies such as diet and exercise will be effective, in light of patient genotype). In addition, for non-human applications, the ability to predict metabolic syndrome, obesity predisposition and insulin resistance is useful, e.g., to livestock breeders who wish to perform marker-assisted breeding (by conventional or in vitro fertilization (IVF) assisted methods) to control, e.g., fat production in livestock. Thus, where the organism is a non-human mammal, the methods optionally further include selecting the non-human mammal, or germplasm (e.g., sperm or eggs) therefrom, from a population of non-human mammals, based upon the determined correlation to phenotype. The resulting selected non-human mammal can be bred with another non-human mammal (by conventional or IVF assisted methods) to optimize genotype and resulting phenotype in one or more offspring.

Kits that comprise, e.g., probes for identifying the markers herein, e.g., packaged in suitable containers with instructions for correlating detected alleles to a treatment emergent weight gain phenotype, metabolic syndrome phenotype, an insulin resistance phenotype, or an obesity predisposition phenotype are a feature of the invention as well.

In an additional aspect, methods of identifying modulators of a treatment emergent weight gain phenotype, a metabolic syndrome phenotype, an insulin resistance phenotype, or an obesity predisposition phenotype, are provided. The methods include contacting a potential modulator to a gene or gene product, such as a gene product corresponding to PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2, and/or any gene product in Appendix 1, and/or a gene corresponding to any of these gene products. An effect of the potential modulator on the gene or gene product is detected, thereby identifying whether the potential modulator modulates the treatment emergent weight gain phenotype, the metabolic syndrome phenotype, the insulin resistance phenotype, or the obesity predisposition phenotype. All of the features described above for the alleles, genes, markers, etc., are applicable to these methods as well.

Effects of interest for which one may screen include: (a) increased or decreased expression of PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 and/or any gene product of Appendix 1, in the presence of the modulator; (b) a change in the timing or location of expression of PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 and/or any gene product in Appendix 1 in the presence of the modulator; (c) a change in localization of proteins encoded by the genes for PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 or others in Appendix 1 in the presence of the modulator; (d) an increased or decreased cleavage of IGFBP4 by PAPPA in the presence of the modulator; (e) increased or decreased catalysis of peptide cleavage by PAM; (f) a change in function of cilia comprising pf20 and/or DNAH11; (g) a change in association (affinity, etc.) of a PKD1 gene product, e.g., polycystin-1, with PKD2 gene product, e.g., polycystin-2; (h) a change in localization of polycystin-2 in or to a plasma membrane; (i) a change in activity of a channel comprising a polycystin-1; (j) a change in localization of a KCNMA1 gene product; and/or (k) a change in activity of a channel comprising KCNMA1 gene product.

The invention also includes kits for treatment of a treatment emergent weight gain phenotype, a metabolic syndrome phenotype, an obesity predisposition phenotype or an insulin resistance phenotype. In one aspect, the kit comprises a modulator identified by the method above and instructions for administering the compound to a patient to treat the metabolic syndrome phenotype, treatment emergent weight gain phenotype, obesity predisposition phenotype or an insulin resistance phenotype.

In an additional aspect, systems for identifying a treatment emergent weight gain phenotype, a metabolic syndrome phenotype, an insulin resistance phenotype, or an obesity predisposition phenotype for an organism or biological sample derived therefrom are provided. Such systems include, e.g., a set of marker probes or primers configured to detect at least one allele of one or more gene or linked locus associated with the treatment emergent weight gain phenotype, the insulin resistance phenotype, the obesity predisposition phenotype or the metabolic syndrome phenotype, wherein the gene comprises or encodes PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 and/or any gene or gene product of Appendix 1. Typically, the set of marker probes or primers can include or detect a nucleotide sequence of Appendix 1, or an allele closely linked thereto. The system typically also includes a detector that is configured to detect one or more signal outputs (e.g., light emissions) from the set of marker probes or primers, or an amplicon produced from the set of marker probes or primers, thereby identifying the presence or absence of the allele. System instructions that correlate the presence or absence of the allele with the predicted metabolic syndrome phenotype, the insulin resistance phenotype, or the obesity predisposition phenotype, thereby identifying the metabolic syndrome phenotype, the insulin resistance phenotype, or the obesity predisposition phenotype for the organism or biological sample derived therefrom are also a feature of the system. The instructions can include at least one look-up table that includes a correlation between the presence or absence of the one or more alleles and the insulin resistance or obesity predisposition. The system can further include a sample, which is typically derived from a mammal, including e.g., a genomic DNA, an amplified genomic DNA, a cDNA, an amplified cDNA, RNA, or an amplified RNA.

It will be appreciated that the methods, systems and kits above can all be used together in various combinations and that features of the methods can be reflected in the systems and kits, and vice-versa.

### BRIEF DESCRIPTION OF THE FIGURES

FIG. 1 provides a graph of treatment emergent weight gain distribution, in which the BMI (body mass index) change is charted against the total patient population, including the 20% lowest gainers (n=258) and the 20% highest gainers (N=255).

FIG. 2 shows a schematic overview of a whole genome association study.

FIG. 3 shows representative scatter plots for PKHD1 and PAM, two of the genes identified as having SNPs that correlate with weight gain in the second phase study, with p value on the y-axis and the position that a given SNP maps to within the gene on the x-axis.

FIG. 4 provides a schematic outline of an overall Zyprexa (olanzapine) whole genome scan study.

### DETAILED DESCRIPTION OF THE INVENTION

The present invention provides correlations between polymorphisms in or proximal to the genes for PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 or any other gene or locus in Appendix 1 and treatment emergent weight gain, metabolic syndrome, obesity predisposition and/or insulin resistance. Thus, detection of particular polymorphisms in these loci, genes or gene products provides methods for identifying patients that have or are at risk for metabolic syndrome, obesity predisposition and/or insulin resistance. Systems for detecting and correlating alleles to treatment emergent weight gain, metabolic syndrome, obesity predisposition and/or insulin resistance, e.g., for practicing the methods, are also a feature of the invention. In addition, the identification of these polymorphisms provides high-throughput systems and methods for identifying modulators of treatment emergent weight gain, metabolic syndrome, obesity predisposition and/or insulin resistance.

The following definitions are provided to more clearly identify aspects of the present invention. They should not be imputed to any other related or unrelated application or patent.

### DEFINITIONS

It is to be understood that this invention is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a,” “an” and “the,” for example, optionally include plural referents unless the content clearly dictates otherwise. Thus, for example, reference to “a probe” optionally includes a plurality of probe molecules; similarly, depending on the context, use of the term “a nucleic acid” optionally includes, as a practical matter, many copies of that nucleic acid molecule. Letter designations for genes or proteins can refer to the gene form and/or the protein form, depending on context. One of skill is fully able to relate the nucleic acid and amino acid forms of the relevant biological molecules by reference to the sequences herein, known sequences and the genetic code.

Unless otherwise indicated, nucleic acids are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

A “phenotype” is a trait or collection of traits that is/are observable in an individual or population. The trait can be quantitative (a quantitative trait, or QTL) or qualitative.

A “metabolic syndrome phenotype” is a phenotype that displays a predisposition towards developing metabolic syndrome in an individual, or that displays metabolic syndrome in the individual. A phenotype that displays a predisposition for metabolic syndrome, can for example, show a higher likelihood that the syndrome will develop in an individual with the phenotype than in members of the general population under a given set of environmental conditions, such as a high calorie, e.g., high-fat, and/or high-carbohydrate diet, and/or a low physical activity regime. Metabolic syndrome can be characterized by any of a number of metabolic disorders or risk factors, generally considered to most typify metabolic syndrome when more than one of these factors are present in a single individual. The factors include: central obesity (disproportionate fat tissue in and around the abdomen), atherogenic dyslipidemia (these include a family of blood fat disorders including, e.g., high triglycerides and low HDL cholesterol, that can foster plaque buildups in the vascular system, including artery walls), high blood pressure (e.g., 130/85 mmHg or higher), insulin resistance or glucose intolerance (the body can't properly use insulin or blood sugar), a chronic prothrombotic state (e.g., characterized by high fibrinogen or plasminogen activator inhibitor [−1] levels in the blood), and a chronic proinflammatory state (e.g., characterized by higher than normal levels of high-sensitivity C-reactive protein in the blood).

An “insulin resistance phenotype” is a phenotype that displays a predisposition for developing insulin resistance in an individual or that display insulin resistance in the individual. For example, an individual with the phenotype can show a higher likelihood that the syndrome will develop in the individual than in members of the general population under a given set of environmental conditions (e.g., those noted above for metabolic syndrome). Any of a variety of tests in current use can be used to determine insulin resistance, including: the Oral Glucose Tolerance Test (OGTT), Fasting Blood Glucose (FBG), Normal Glucose Tolerance (NGT), Impaired Glucose Tolerance (IGT), Impaired Fasting Glucose (IFG), Homeostasis Model Assessment (HOMA), the Quantitative Insulin Sensitivity Check Index (QUICKI) and the Intravenous Insulin Tolerance Test (IVITT). See also, www.retroconference.org/2002/Posters/12814.pdf; De Vegt (1998) “The 1997 American Diabetes Association criteria versus the 1985 World Health Organization criteria for the diagnosis of abnormal glucose tolerance: poor agreement in the Hoorn Study.” Diab Care 1998, 21:1686-1690; Matthews (1985) “Homeostasis model assessment: insulin resistance and B-cell function from fasting plasma glucose and insulin concentrations in man.” Diabetologia 28:412-419; Katz, A (2000) “Quantitative Insulin Sesitivity Check Index: A Simple, Accurate Method for Assessing Insulin Sensitivity In Humans.” JCE & M 85:2402-2410. It will be appreciated that patients with insulin resistance can also suffer from metabolic syndrome and/or obesity.

An “obesity predisposition phenotype” is a phenotype that displays a predisposition for developing obesity (e.g., central obesity) in an individual, or that displays obesity. For example, an individual with the phenotype can show a higher likelihood that obesity will develop in the individual than in members of the general population under a given set of environmental conditions (e.g., those noted above for metabolic syndrome). “Central obesity” is a trait characterized by a large and/or disproportionate deposit of fat around the waist. Most women with a waist of greater than 35 inches, and most men with a waist of greater than 40 inches are classified as having central obesity. It will be appreciated that patients with metabolic syndrome are often obese, and/or insulin resistant; the three phenotypes are all interrelated.

A “treatment emergent weight gain phenotype” is a phenotype that displays a predisposition towards weight gain when a patient having the phenotype is undergoing a specified treatment. For example, a patient undergoing any of a variety of drug therapies, e.g., treatment with an a typical antipsychotic medication, e.g., olanzapine, during anti-psychotic drug therapy, can display a predisposition towards weight gain.

A “polymorphism” is a locus that is variable; that is, within a population, the nucleotide sequence at a polymorphism has more than one version or allele. The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. One example of a polymorphism is a “single nucleotide polymorphism” (SNP), which is a polymorphism at a single nucleotide position in a genome (the nucleotide at the specified position varies between individuals or populations).

An allele “positively” correlates with a trait when it is linked to it and when presence of the allele is an indictor that the trait or trait form will occur in an individual comprising the allele. An allele negatively correlates with a trait when it is linked to it and when presence of the allele is an indicator that a trait or trait form will not occur in an individual comprising the allele.

A marker polymorphism or allele is “correlated” with a specified phenotype (metabolic syndrome, obesity predisposition, insulin resistance, etc.) when it can be statistically linked (positively or negatively) to the phenotype. This correlation is often inferred as being causal in nature, but it need not be—simple genetic linkage to (association with) a locus for a trait that underlies the phenotype is sufficient.

A “favorable allele” is an allele at a particular locus that positively correlates with a desirable phenotype, e.g., resistance to obesity, or resistance to metabolic syndrome, or that negatively correlates with an undesirable phenotype, e.g., an allele that negatively correlates with obesity predisposition or predisposition to metabolic syndrome. The desired phenotype can, of course, vary, e.g., in some animal breeding contexts, predisposition to obesity can be desirable, instead of undesirable, as it is in many human populations. A favorable allele of a linked marker is a marker allele that segregates with the favorable allele. A favorable allelic form of a chromosome segment is a chromosome segment that includes a nucleotide sequence that positively correlates with the desired phenotype, or that negatively correlates with the unfavorable phenotype at one or more genetic loci physically located on the chromosome segment.

An “unfavorable allele” is an allele at a particular locus that negatively correlates with a desirable phenotype, or that correlates positively with an undesirable phenotype, e.g., positive correlation to obesity predisposition, or metabolic syndrome predisposition, or negative correlation with obesity resistance or resistance to metabolic syndrome. Here again, the desired phenotype can, of course, vary, e.g., in some animal breeding contexts, predisposition to obesity can be desirable, instead of undesirable, as it is in many human populations. An unfavorable allele of a linked marker is a marker allele that segregates with the unfavorable allele. An unfavorable allelic form of a chromosome segment is a chromosome segment that includes a nucleotide sequence that negatively correlates with the desired phenotype, or positively correlates with the undesirable phenotype at one or more genetic loci physically located on the chromosome segment.

“Allele frequency” refers to the frequency (proportion or percentage) at which an allele is present at a locus within an individual, within a line, or within a population of lines. For example, for an allele “A,” diploid individuals of genotype “AA,” “Aa,” or “aa” have allele frequencies of 1.0, 0.5, or 0.0, respectively. One can estimate the allele frequency within a line or population by averaging the allele frequencies of a sample of individuals from that line or population. Similarly, one can calculate the allele frequency within a population of lines by averaging the allele frequencies of lines that make up the population.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

A “locus” is a chromosomal position or region. For example, a polymorphic locus is a position or region where a polymorphic nucleic acid, trait determinant, gene or marker is located. In a further example, a “gene locus” is a specific chromosome location in the genome of a species where a specific gene can be found. Similarly, the term “quantitative trait locus” or “QTL” refers to a locus with at least two alleles that differentially affect the expression or alter the variation of a quantitative or continuous phenotypic trait in at least one genetic background, e.g., in at least one breeding population or progeny.

A “marker,” “molecular marker” or “marker nucleic acid” refers to a nucleotide sequence or encoded product thereof (e.g., a protein) used as a point of reference when identifying a locus or a linked locus. A marker can be derived from genomic nucleotide sequence or from expressed nucleotide sequences (e.g., from an RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Nucleic acids are “complementary” when they specifically hybridize in solution, e.g., according to Watson-Crick base pairing rules. A “marker locus” is a locus that can be used to track the presence of a second linked locus, e.g., a linked or correlated locus that encodes or contributes to the population variation of a phenotypic trait. For example, a marker locus can be used to monitor segregation of alleles at a locus, such as a QTL, that are genetically or physically linked to the marker locus. Thus, a “marker allele,” or, alternatively, an “allele of a marker locus” is one of a plurality of polymorphic nucleotide sequences found at a marker locus in a population that is polymorphic for the marker locus. In one aspect, the present invention provides marker loci correlating with a phenotype of interest, e.g., treatment emergent weight gain/obesity predisposition/insulin resistance/metabolic syndrome. Each of the identified markers is expected to be in close or overlapping physical and genetic proximity (resulting in physical and/or genetic linkage ) to a genetic element, e.g., a QTL, that contributes to the relevant phenotype. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of allele specific hybridization (ASH), detection of single nucleotide extension, detection of amplified variable sequences of the genome, detection of self-sustained sequence replication, detection of simple sequence repeats (SSRs), detection of single nucleotide polymorphisms (SNPs), or detection of amplified fragment length polymorphisms (AFLPs).

A “genetic map” is a description of genetic linkage (or association) relationships among loci on one or more chromosomes (or linkage groups) within a given species, generally depicted in a diagrammatic or tabular form. “Mapping” is the process of defining the linkage relationships of loci through the use of genetic markers, populations segregating for the markers, and standard genetic principles of recombination frequency. A “map location” is an assigned location on a genetic map relative to linked genetic markers where a specified marker can be found within a given species. The term “chromosome segment” or designates a contiguous linear span of genomic DNA that resides on a single chromosome. Similarly, a “haplotype” is a set of genetic loci found in the heritable material of an individual or population (the set can be a contiguous or non-contiguous). In the context of the present invention genetic elements such as one or more alleles herein and one or more linked marker alleles can be located within a chromosome segment and are also, accordingly, genetically linked, a specified genetic recombination distance of less than or equal to 20 centimorgan (cM) or less, e.g., 15 cM or less, often 10 cM or less, e.g., about 9, 8, 7, 6, 5, 4, 3, 2, 1, 0.75, 0.5, 0.25, or 0.1 CM or less. That is, two closely linked genetic elements within a single chromosome segment undergo recombination during meiosis with each other at a frequency of less than or equal to about 20%, e.g., about 19%, 18%, 17%, 16%, 15%, 14%, 13%, 12%, 11%, 10%, 9%, 8%, 7%, 6%, 5%, 4%, 3%, 2%, 1%, 0.75%, 0.5%, 0.25%, or 0.1% or less.

A “genetic recombination frequency” is the frequency of a recombination event between two genetic loci. Recombination frequency can be observed by following the segregation of markers and/or traits during meiosis. In the context of this invention, a marker locus is “associated with” another marker locus or some other locus (for example, an obesity or metabolic syndrome locus), when the relevant loci are part of the same linkage group due to association and are in linkage disequilibrium. This occurs when the marker locus and a linked locus are found together in progeny more frequently than if the loci segregate randomly. Similarly, a marker locus can also be associated with a trait, e.g., a marker locus can be “associated with” a given trait when the marker locus is in linkage disequilibrium with the trait. The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random frequency (in the case of co-segregating traits, the loci that underlie the traits are in sufficient proximity to each other). Linked loci co-segregate more than 50% of the time, e.g., from about 51% to about 100% of the time. Advantageously, the two loci are located in close proximity such that recombination between homologous chromosome pairs does not occur between the two loci during meiosis with high frequency, e.g., such that closely linked loci co-segregate at least about 80% of the time, more preferably at least about 85% of the time, still more preferably at least 90% of the time, e.g., 91%, 92%, 93%, 94%, 95%, 96%, 97%, 98%, 99%, 99.5%, 99.75%, or 99.90% or more of the time.

The phrase “closely linked,” in the present application, means that recombination between two linked loci (e.g., a SNP such as one identified in Appendix 1 herein and a second linked allele) occurs with a frequency of equal to or less than about 20%. Put another way, the closely (or “tightly”) linked loci co-segregate at least 80% of the time. Marker loci are especially useful in the present invention when they are closely linked to target loci (e.g., QTL for metabolic syndrome, obesity predisposition, and/or insulin resistance, or, alternatively, simply other marker loci). The more closely a marker is linked to a target locus, the better an indicator for the target locus that the marker is. Thus, in one embodiment, tightly linked loci such as a marker locus and a second locus display an inter-locus recombination frequency of about 20% or less, e.g., 15% or less, e.g., 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus such as a QTL) display a recombination frequency of about 1% or less, e.g., about 0.75% or less, more preferably about 0.5% or less, or yet more preferably about 0.25% or less, or still more preferably about 0.1% or less. Two loci that are localized to the same chromosome, and at such a distance that recombination between the two loci occurs at a frequency of less than about 20%, e.g., 15%, more preferably 10% (e.g., about 9%, 8%, 7%, 6%, 5%, 4%, 3%, 2%, 1%, 0.75%, 0.5%, 0.25%, 0.1% or less) are also said to be “proximal to” each other. When referring to the relationship between two linked genetic elements, such as a genetic element contributing to a trait and a proximal marker, “coupling” phase linkage indicates the state where the “favorable” allele at the trait locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for obesity or metabolic syndrome) is physically associated on the same chromosome strand as an “unfavorable” allele at the proximal marker locus, and the two “favorable” alleles are not inherited together (i.e., the two loci are “out of phase” with each other).

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods. An “amplicon” is an amplified nucleic acid, e.g., a nucleic acid that is produced by amplifying a template nucleic acid by any available amplification method (e.g., PCR, LCR, transcription, or the like).

A “genomic nucleic acid” is a nucleic acid that corresponds in sequence to a heritable nucleic acid in a cell. Common examples include nuclear genomic DNA and amplicons thereof. A genomic nucleic acid is, in some cases, different from a spliced RNA, or a corresponding cDNA, in that the spliced RNA or cDNA is processed, e.g., by the splicing machinery, to remove introns. Genomic nucleic acids optionally comprise non-transcribed (e.g., chromosome structural sequences, promoter regions, enhancer regions, etc.) and/or non-translated sequences (e.g., introns), whereas spliced RNA/cDNA typically do not have non-transcribed sequences or introns. A “template genomic nucleic acid” is a genomic nucleic acid that serves as a template in an amplification reaction (e.g., a polymerase based amplification reaction such as PCR, a ligase mediated amplification reaction such as LCR, a transcription reaction, or the like).

An “exogenous nucleic acid” is a nucleic acid that is not native to a specified system (e.g., a germplasm, cell, individual, etc.), with respect to sequence, genomic position, or both. As used herein, the terms “exogenous” or “heterologous” as applied to polynucleotides or polypeptides typically refers to molecules that have been artificially supplied to a biological system (e.g., a cell, an individual, etc.) and are not native to that particular biological system. The terms can indicate that the relevant material originated from a source other than a naturally occurring source, or can refer to molecules having a non-natural configuration, genetic location or arrangement of parts.

The term “introduced” when referring to translocating a heterologous or exogenous nucleic acid into a cell refers to the incorporation of the nucleic acid into the cell using any methodology. The term encompasses such nucleic acid introduction methods as “transfection,” “transformation” and “transduction.”

As used herein, the term “vector” is used in reference to polynucleotides or other molecules that transfer nucleic acid segment(s) into a cell. The term “vehicle” is sometimes used interchangeably with “vector.” A vector optionally comprises parts which mediate vector maintenance and enable its intended use (e.g., sequences necessary for replication, genes imparting drug or antibiotic resistance, a multiple cloning site, operably linked promoter/enhancer elements which enable the expression of a cloned gene, etc.). Vectors are often derived from plasmids, bacteriophages, or plant or animal viruses. A “cloning vector” or “shuttle vector” or “subcloning vector” contains operably linked parts that facilitate subcloning steps (e.g., a multiple cloning site containing multiple restriction endonuclease sites).

The term “expression vector” as used herein refers to a vector comprising operably linked polynucleotide sequences that facilitate expression of a coding sequence in a particular host organism (e.g., a bacterial expression vector or a mammalian cell expression vector). Polynucleotide sequences that facilitate expression in prokaryotes typically include, e.g., a promoter, an operator (optional), and a ribosome binding site, often along with other sequences. Eukaryotic cells can use promoters, enhancers, termination and polyadenylation signals and other sequences that are generally different from those used by prokaryotes.

A specified nucleic acid is “derived from” a given nucleic acid when it is constructed using the given nucleic acid's sequence, or when the specified nucleic acid is constructed using the given nucleic acid.

A “gene” is one or more sequence(s) of nucleotides in a genome that together encode one or more expressed molecule, e.g., an RNA, or polypeptide. The gene can include coding sequences that are transcribed into RNA which may then be translated into a polypeptide sequence, and can include associated structural or regulatory sequences that aid in replication or expression of the gene. Genes of interest in the present invention include genomic sequences that encode, e.g.: PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 or any gene or gene product in Appendix 1.

A “genotype” is the genetic constitution of an individual (or group of individuals) at one or more genetic loci. Genotype is defined by the allele(s) of one or more known loci of the individual, typically, the compilation of alleles inherited from its parents. A “haplotype” is the genotype of an individual at a plurality of genetic loci on a single DNA strand. Typically, the genetic loci described by a haplotype are physically and genetically linked, i.e., on the same chromosome strand.

A “set” of markers or probes refers to a collection or group of markers or probes, or the data derived therefrom, used for a common purpose, e.g., identifying an individual with a specified phenotype (e.g., treatment emergent weight gain, obesity predisposition, metabolic syndrome disorder, etc.). Frequently, data corresponding to the markers or probes, or derived from their use, is stored in an electronic medium. While each of the members of a set possess utility with respect to the specified purpose, individual markers selected from the set as well as subsets including some, but not all of the markers, are also effective in achieving the specified purpose.

A “look up table” is a table that correlates one form of data to another, or one or more forms of data with a predicted outcome to which the data is relevant. For example, a look up table can include a correlation between allele data and a predicted trait that an individual comprising one or more given alleles is likely to display. These tables can be, and typically are, multidimensional, e.g., taking multiple alleles into account simultaneously, and, optionally, taking other factors into account as well, such as genetic background, e.g., in making a trait prediction.

A “computer readable medium” is an information storage media that can be accessed by a computer using an available or custom interface. Examples include memory (e.g., ROM or RAM, flash memory, etc.), optical storage media (e.g., CD-ROM), magnetic storage media (computer hard drives, floppy disks, etc.), punch cards, and many others that are commercially available. Information can be transmitted between a system of interest and the computer, or to or from the computer or to or from the computer readable medium for storage or access of stored information. This transmission can be an electrical transmission, or can be made by other available methods, such as an IR link, a wireless connection, or the like.

“System instructions” are instruction sets that can be partially or fully executed by the system. Typically, the instruction sets are present as system software.

A “translation product” is a product (typically a polypeptide) produced as a result of the translation of a nucleic acid. A “transcription product” is a product (e.g., an RNA, such as an mRNA, a catalytic or biologically active RNA, or the like) produced as a result of transcription of a nucleic acid (e.g., a DNA).

An “array” is an assemblage of elements. The assemblage can be spatially ordered (a “patterned array”) or disordered (a “randomly patterned” array). The array can form or comprise one or more functional elements (e.g., a probe region on a microarray) or it can be non-functional.

As used herein, the term “SNP” or “single nucleotide polymorphism” refers to a genetic variation between individuals; e.g., a single nitrogenous base position in the DNA of organisms that is variable. As used herein, “SNPs” is the plural of SNP. Of course, when one refers to DNA herein, such reference may include derivatives of the DNA such as amplicons, RNA transcripts thereof, etc.

Overview

The invention includes new correlations between the genes or linked loci for PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 and/or the genes, products or loci of Appendix 1 and a variety of related metabolic disorders, including metabolic syndrome, obesity predisposition, insulin resistance and treatment emergent weight gain. Certain alleles in, and linked to, these genes or gene products are predictive of the likelihood that an individual possessing the relevant alleles will develop one or more of these metabolic disorders. Accordingly, detection of these alleles, by any available method, can be used for diagnostic purposes such as early detection of susceptibility to a metabolic disorder, prognosis for patients that present with the metabolic disorder, and in assisting diagnosis, e.g., where current criteria are insufficient for a definitive diagnosis. In addition, because fat production in livestock is important to livestock breeders, it is possible to perform marker assisted selection (MAS) on livestock and livestock germplasm using such allele correlations to select for or against obese phenotypes.

The identification that the genes for PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 and/or the genes or gene products of Appendix 1 are correlated to the metabolic disorders noted above also provides a platform for screening potential modulators of metabolic disorders. Modulators of the activity of any of these genes or their encoded proteins are expected to have an effect on treatment emergent weight gain, metabolic syndrome, obesity predisposition, and insulin resistance. Thus, methods of screening, systems for screening and the like, are features of the invention. Modulators identified by these screening approaches are also a feature of the invention.

Kits for the diagnosis and treatment of treatment emergent weight gain, metabolic syndrome, e.g., comprising probes to identify relevant alleles, packaging materials, and instructions for correlating detection of relevant alleles to metabolic diseases are also a feature of the invention. These kits can also include modulators of the relevant disease and/or instructions for treating patients using conventional methods.

Methods of Identifying Treatment Emergency Weight Gain Metabolic Syndrome, Insulin Resistance, and Obesity Predisposition

As noted, the invention provides the discovery that certain genes or other loci (e.g., PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 and/or the genes or loci of Appendix 1), are linked to treatment emergent weight gain, metabolic syndrome, insulin resistance, obesity predisposition and other related phenotypes. Thus, by detecting markers (e.g., the SNPs in Appendix 1/Table 3B or loci closely linked thereto) that correlate, positively or negatively, with the relevant phenotypes, it can be determined whether an individual or population is likely to be susceptible to these phenotypes. This provides enhanced early detection options to identify patients that are likely to eventually suffer from these phenotypes, making it possible, in some cases, to prevent actual development of treatment emergent weight gain, metabolic syndrome, obesity, diabetes, etc., e.g., by taking early preventative action (e.g., any existing therapy such as diet, exercise, available medications, etc.). In addition, use of the various markers herein also adds certainty to existing diagnostic techniques for identifying whether a patient is suffering from, e.g., metabolic syndrome, which can be somewhat ambiguous using previous methods, e.g., as discussed in the Background of the Invention, above. Furthermore, knowledge of whether there is a molecular basis for obesity, metabolic syndrome, insulin resistance, etc., can also assist in determining patient prognosis, e.g., by providing an indication of how likely it is that a patient can respond to conventional therapy for the relevant disorder, or whether more serious options such as gastric surgery are likely to be necessary. Disease treatment can also be targeted based on what type of molecular disorder the patient displays.

In non-human subjects (e.g., non-human mammals such as livestock), it is also possible to use this information both for disease diagnosis and prevention (e.g., treatment of pets such as dogs and cats, etc.). as in humans. In addition, it is possible to perform marker-assisted animal breeding to enhance either fat production or lean meat production, depending on what is desired. In brief, livestock animals or germplasm can be selected for marker alleles that positively or negatively correlate with treatment emergent weight gain, metabolic syndrome, insulin resistance, and/or obesity predisposition, without actually raising the livestock and measuring for the desired trait. Marker assisted selection (MAS) is a powerful shortcut to selecting for desired phenotypes and for introgressing desired traits into livestock herds (e.g., introgressing desired traits into elite herd populations). MAS is easily adapted to high throughput molecular analysis methods that can quickly screen genetic material for the markers of interest, and is much more cost effective than raising and observing livestock for visible traits.

Detection methods for detecting relevant alleles can include any available method, e.g., amplification technologies. For example, detection can include amplifying the polymorphism or a sequence associated therewith and detecting the resulting amplicon. This can include admixing an amplification primer or amplification primer pair with a nucleic acid template isolated from the organism or biological sample (e.g., comprising the SNP or other polymorphism), e.g., where the primer or primer pair is complementary or partially complementary to at least a portion of the gene or tightly linked polymorphism, or to a sequence proximal thereto. The primer is typically capable of initiating nucleic acid polymerization by a polymerase on the nucleic acid template. The primer or primer pair is extended, e.g., in a DNA polymerization reaction (PCR, RT-PCR, etc.) comprising a polymerase and the template nucleic acid to generate the amplicon. The amplicon is detected by any available detection process, e.g., sequencing, hybridizing the amplicon to an array (or affixing the amplicon to an array and hybridizing probes to it), digesting the amplicon with a restriction enzyme (e.g., RFLP), real-time PCR analysis, single nucleotide extension, allele-specific hybridization, or the like.

The correlation between a detected polymorphism and a trait can be performed by any method that can identify a relationship between an allele and a phenotype. Most typically, these methods involve referencing a look up table that comprises correlations between alleles of the polymorphism and the phenotype. The table can include data for multiple allele-phenotype relationships and can take account of additive or other higher order effects of multiple allele-phenotype relationships, e.g., through the use of statistical tools such as principle component analysis, heuristic algorithms, etc.

Within the context of these methods, the following discussion first focuses on how markers and alleles are linked and how this phenomenon can be used in the context of methods for identifying treatment emergent weight gain, metabolic syndrome, insulin resistance, and/or obesity predisposition, and then focuses on marker detection methods. Additional sections below discuss data analysis.

Markers, Linkage And Alleles

In traditional linkage (or association) analysis, no direct knowledge of the physical relationship of genes on a chromosome is required. Mendel's first law is that factors of pairs of characters are segregated, meaning that alleles of a diploid trait separate into two gametes and then into different offspring. Classical linkage analysis can be thought of as a statistical description of the relative frequencies of cosegregation of different traits. Linkage analysis is the well characterized descriptive framework of how traits are grouped together based upon the frequency with which they segregate together. That is, if two non-allelic traits are inherited together with a greater than random frequency, they are said to be “linked.” The frequency with which the traits are inherited together is the primary measure of how tightly the traits are linked, i.e., traits which are inherited together with a higher frequency are more closely linked than traits which are inherited together with lower (but still above random) frequency. Traits are linked because the genes which underlie the traits reside near one another on the same chromosome. The further apart on a chromosome the genes reside, the less likely they are to segregate together, because homologous chromosomes recombine during meiosis. Thus, the further apart on a chromosome the genes reside, the more likely it is that there will be a recombination event during meiosis that will result in two genes segregating separately into progeny.

A common measure of linkage (or association) is the frequency with which traits cosegregate. This can be expressed as a percentage of cosegregation (recombination frequency) or, also commonly, in centiMorgans (cM), which are actually a reciprocal unit of recombination frequency. The cM is named after the pioneering geneticist Thomas Hunt Morgan and is a unit of measure of genetic recombination frequency. One cM is equal to a 1% chance that a trait at one genetic locus will be separated from a trait at another locus due to recombination in a single generation (meaning the traits segregate together 99% of the time). Because chromosomal distance is approximately proportional to the frequency of recombination events between traits, there is an approximate physical distance that correlates with recombination frequency. For example, in humans, 1 cM correlates, on average, to about 1 million base pairs (1 Mbp).

Marker loci are themselves traits and can be assessed according to standard linkage analysis by tracking the marker loci during segregation. Thus, in the context of the present invention, one cM is equal to a 1% chance that a marker locus will be separated from another locus (which can be any other trait, e.g., another marker locus, or another trait locus that encodes a QTL for treatment emergent weight gain, metabolic syndrome, insulin resistance, and/or obesity predisposition), due to recombination in a single generation. The markers herein, e.g., those listed in Appendix 1, can correlate with treatment emergent weight gain, metabolic syndrome, insulin resistance, and/or obesity predisposition. This means that the markers comprise or are sufficiently proximal to a QTL for treatment emergent weight gain, metabolic syndrome, insulin resistance, and/or obesity predisposition that they can be used as a predictor for the trait itself. This is extremely useful in the context of disease diagnosis and, in livestock applications, for marker assisted selection (MAS).

From the foregoing, it is clear that any marker that is linked to a trait locus of interest (e.g., in the present case, a QTL or identified linked marker locus for treatment emergent weight gain, metabolic syndrome, insulin resistance, and/or obesity predisposition, e.g., as in Appendix 1) can be used as a marker for that trait. Thus, in addition to the markers noted in Appendix 1, other markers closely linked to the markers itemized in Appendix 1 can also usefully predict the presence of the marker alleles indicated in Appendix 1 (and, thus, the relevant phenotypic trait). Such linked markers are particularly useful when they are sufficiently proximal to a given locus so that they display a low recombination frequency with the given locus. In the present invention, such closely linked markers are a feature of the invention. Closely linked loci display a recombination frequency with a given marker of about 20% or less (the given marker is within 20 cM of the given marker). Put another way, closely linked loci co-segregate at least 80% of the time. More preferably, the recombination frequency is 10% or less, e.g., 9%, 8%, 7%, 6%, 5%, 4%, 3%, 2%, 1%, 0.5%, 0.25%, or 0.1% or less. In one typical class of embodiments, closely linked loci are within 5 cM or less of each other.

As one of skill in the art will recognize, recombination frequencies (and, as a result, map positions) can vary depending on the map used (and the markers that are on the map). Additional markers that are closely linked to (e.g., within about 20 cM, or more preferably within about 10 cM of) the markers identified in Appendix 1 may readily be used for identification of QTL for treatment emergent weight gain, metabolic syndrome, insulin resistance, and/or obesity predisposition.

Marker loci are especially useful in the present invention when they are closely linked to target loci (e.g., QTL for treatment emergent weight gain, metabolic syndrome, insulin resistance, and/or obesity predisposition, or, alternatively, simply other marker loci, such as those itemized in Appendix 1 that are, themselves linked to such QTL) that they are being used as markers for. The more closely a marker is linked to a target locus that encodes or affects a phenotypic trait, the better an indicator for the target locus that the marker is (due to the reduced cross-over frequency between the target locus and the marker). Thus, in one embodiment, closely linked loci such as a marker locus and a second locus (e.g., a given marker locus of Appendix 1 and an additional second locus) display an inter-locus cross-over frequency of about 20% or less, e.g., 15% or less, preferably 10% or less, more preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus such as a QTL) display a recombination a frequency of about 1% or less, e.g., about 0.75% or less, more preferably about 0.5% or less, or yet more preferably about 0.25% or 0.1% or less. Thus, the loci are about 20 cM, 19 cM, 18 cM, 17 cM, 16 cM, 15 cM, 14 cM, 13 cM, 12 cM, 11 cM, 10 cM, 9 cM, 8 cM, 7 cM, 6 cM, 5 cM, 4 cM, 3 cM, 2 cM, 1 cM, 0.75 cM, 0.5 cM, 0.25 cM, 0 or 0.1 cM or less apart. Put another way, two loci that are localized to the same chromosome, and at such a distance that recombination between the two loci occurs at a frequency of less than 20% (e.g., about 19%, 18%, 17%, 16%, 15%, 14%, 13%, 12%, 11%, 10%, 9%, 8%, 7%, 6%, 5%, 4%, 3%, 2%, 1%, 0.75%, 0.5%, 0.25%, 0.1% or less) are said to be “proximal to” each other. In one aspect, linked markers are within 100 kb (which correlates in humans to about 0.1 cM, depending on local recombination rate), e.g., 50 kb, or even 20 kb or less of each other.

When referring to the relationship between two genetic elements, such as a genetic element contributing to treatment emergent weight gain, metabolic syndrome, insulin resistance, and/or obesity predisposition, and a proximal marker, “coupling” phase linkage indicates the state where the “favorable” allele at the locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for treatment emergent weight gain, metabolic syndrome, insulin resistance, and/or obesity predisposition) is physically linked with an “unfavorable” allele at the proximal marker locus, and the two “favorable” alleles are not inherited together (i.e., the two loci are “out of phase” with each other).

In addition to tracking SNP and other polymorphisms in the genome, and in corresponding expressed nucleic acids and polypeptides, expression level differences between individuals or populations for PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2, or the gene products of Appendix 1, in either mRNA or protein form, can also correlate to treatment emergent weight gain, metabolic syndrome, insulin resistance, and/or obesity predisposition phenotypes. Accordingly, markers of the invention can include any of, e.g.: genomic loci, transcribed nucleic acids, spliced nucleic acids, expressed proteins, levels of transcribed nucleic acids, levels of spliced nucleic acids, and levels of expressed proteins.

Marker Amplification Strategies

Amplification primers for amplifying markers (e.g., marker loci) and suitable probes to detect such markers or to genotype a sample with respect to multiple marker alleles, are a feature of the invention. In Appendix 1, specific loci for amplification are provided, along with amplicon sequences that one of skill can easily use (optionally in conjunction with known flanking sequences) in the design of such primers. For example, primer selection for long-range PCR is described in U.S. Ser. No. 10/042,406, filed Jan. 9, 2002 and U.S. Ser. No. 10/236,480, filed Sep. 5, 2002; for short-range PCR, U.S. Ser. No. 10/341,832, filed Jan. 14, 2003 provides guidance with respect to primer selection. Also, there are publicly available programs such as “Oligo” available for primer design. With such available primer selection and design software, the publicly available human genome sequence and the polymorphism locations as provided in Appendix 1, one of skill can design primers to amplify the SNPs of the present invention. Further, it will be appreciated that the precise probe to be used for detection of a nucleic acid comprising a SNP (e.g., an amplicon comprising the SNP) can vary, e.g., any probe that can identify the region of a marker amplicon to be detected can be used in conjunction with the present invention. Further, the configuration of the detection probes can, of course, vary. Thus, the invention is not limited to the sequences recited herein.

Indeed, it will be appreciated that amplification is not a requirement for marker detection—for example, one can directly detect unamplified genomic DNA simply by performing a Southern blot on a sample of genomic DNA. Procedures for performing Southern blotting, standard amplification (PCR, LCR, or the like) and many other nucleic acid detection methods are well established and are taught, e.g., in Sambrook et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)) and PCR Protocols A Guide to Methods and Applications (Innis et al. eds) Academic Press Inc. San Diego, Calif. (1990) (Innis).

Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

Typically, molecular markers are detected by any established method available in the art, including, without limitation, allele specific hybridization (ASH), detection of single nucleotide extension, array hybridization (optionally including ASH), or other methods for detecting single nucleotide polymorphisms (SNPs), amplified fragment length polymorphism (AFLP) detection, amplified variable sequence detection, randomly amplified polymorphic DNA (RAPD) detection, restriction fragment length polymorphism (RFLP) detection, self-sustained sequence replication detection, simple sequence repeat (SSR) detection, single-strand conformation polymorphisms (SSCP) detection, isozyme marker detection, northern analysis (where expression levels are used as markers), quantitative amplification of mRNA or cDNA, or the like. While the exemplary markers provided in the figures and tables herein are SNP markers, any of the aforementioned marker types can be employed in the context of the invention to identify linked loci that affect or effect treatment emergent weight gain, metabolic syndrome, insulin resistance, and/or obesity predisposition.

Example Techniques For Marker Detection

The invention provides molecular markers that comprise or are linked to QTL for treatment emergent weight gain, metabolic syndrome, insulin resistance, and/or obesity predisposition. The markers find use in disease predisposition diagnosis, prognosis, treatment and for marker assisted selection for desired traits in livestock. It is not intended that the invention be limited to any particular method for the detection of these markers.

Markers corresponding to genetic polymorphisms between members of a population can be detected by numerous methods well-established in the art (e.g., PCR-based sequence specific amplification, restriction fragment length polymorphisms (RFLPs), isozyme markers, northern analysis, allele specific hybridization (ASH), array based hybridization, amplified variable sequences of the genome, self-sustained sequence replication, simple sequence repeat (SSR), single nucleotide polymorphism (SNP), random amplified polymorphic DNA (“RAPD”) or amplified fragment length polymorphisms (AFLP). In one additional embodiment, the presence or absence of a molecular marker is determined simply through nucleotide sequencing of the polymorphic marker region. Any of these methods are readily adapted to high throughput analysis.

Some techniques for detecting genetic markers utilize hybridization of a probe nucleic acid to nucleic acids corresponding to the genetic marker (e.g., amplified nucleic acids produced using genomic DNA as a template). Hybridization formats, including, but not limited to: solution phase, solid phase, mixed phase, or in situ hybridization assays are useful for allele detection. An extensive guide to the hybridization of nucleic acids is found in Tijssen (1993) Laboratory Techniques in Biochemistry and Molecular Biology—Hybridization with Nucleic Acid Probes Elsevier, New York, as well as in Sambrook, Berger and Ausubel.

For example, markers that comprise restriction fragment length polymorphisms (RFLP) are detected, e.g., by hybridizing a probe which is typically a sub-fragment (or a synthetic oligonucleotide corresponding to a sub-fragment) of the nucleic acid to be detected to restriction digested genomic DNA. The restriction enzyme is selected to provide restriction fragments of at least two alternative (or polymorphic) lengths in different individuals or populations. Determining one or more restriction enzyme that produces informative fragments for each allele of a marker is a simple procedure, well known in the art. After separation by length in an appropriate matrix (e.g., agarose or polyacrylamide) and transfer to a membrane (e.g., nitrocellulose, nylon, etc.), the labeled probe is hybridized under conditions which result in equilibrium binding of the probe to the target followed by removal of excess probe by washing.

Nucleic acid probes to the marker loci can be cloned and/or synthesized. Any suitable label can be used with a probe of the invention. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radiolabels, enzymes, and colorimetric labels. Other labels include ligands that bind to antibodies labeled with fluorophores, chemiluminescent agents, and enzymes. A probe can also constitute radiolabelled PCR primers that are used to generate a radiolabelled amplicon. Labeling strategies for labeling nucleic acids and corresponding detection strategies can be found, e.g., in Haugland (2003) Handbook of Fluorescent Probes and Research Chemicals Ninth Edition by Molecular Probes, Inc. (Eugene Oreg.). Additional details regarding marker detection strategies are found below.

Amplification-Based Detection Methods

PCR, RT-PCR and LCR are in particularly broad use as amplification and amplification-detection methods for amplifying nucleic acids of interest (e.g., those comprising marker loci), facilitating detection of the nucleic acids of interest. Details regarding the use of these and other amplification methods can be found in any of a variety of standard texts, including, e.g., Sambrook, Ausubel, and Berger. Many available biology texts also have extended discussions regarding PCR and related amplification methods. One of skill will appreciate that essentially any RNA can be converted into a double stranded DNA suitable for restriction digestion, PCR expansion and sequencing using reverse transcriptase and a polymerase (“Reverse Transcription-PCR, or “RT-PCR”). See also, Ausubel, Sambrook and Berger, above. These methods can also be used to quantitatively amplify mRNA or corresponding cDNA, providing an indication of expression levels of mRNA that correspond to PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 or the genes or gene products of Appendix 1 in an individual. Differences in expression levels for these genes between individuals, families, lines and/or populations can be used as markers for treatment emergent weight gain, metabolic syndrome, obesity predisposition and insulin resistance.

Real Time Amplification/Detection Methods

In one aspect, real time PCR or LCR is performed on the amplification mixtures described herein, e.g., using molecular beacons or TaqMan™ probes. A molecular beacon (MB) is an oligonucleotide or PNA which, under appropriate hybridization conditions, self-hybridizes to form a stem and loop structure. The MB has a label and a quencher at the termini of the oligonucleotide or PNA; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, e.g., to a region of an amplicon during amplification), the MB label is unquenched. Details regarding standard methods of making and using MBs are well established in the literature and MBs are available from a number of commercial reagent sources. See also, e.g., Leone et al. (1995) “Molecular beacon probes combined with amplification by NASBA enable homogenous real-time detection of RNA.” Nucleic Acids Res. 26:2150-2155; Tyagi and Kramer (1996) “Molecular beacons: probes that fluoresce upon hybridization” Nature Biotechnology 14:303-308; Blok and Kramer (1997) “Amplifiable hybridization probes containing a molecular switch” Mol Cell Probes 11:187-194; Hsuih et al. (1997) “Novel, ligation-dependent PCR assay for detection of hepatitis C in serum” J Clin Microbiol 34:501-507; Kostrikis et al. (1998) “Molecular beacons: spectral genotyping of human alleles” Science 279:1228-1229; Sokol et al. (1998) “Real time detection of DNA:RNA hybridization in living cells” Proc. Natl. Acad. Sci. U.S.A. 95:11538-11543; Tyagi et al. (1998) “Multicolor molecular beacons for allele discrimination” Nature Biotechnology 16:49-53; Bonnet et al. (1999) “Thermodynamic basis of the chemical specificity of structured DNA probes” Proc. Natl. Acad. Sci. U.S.A. 96:6171-6176; Fang et al. (1999) “Designing a novel molecular beacon for surface-immobilized DNA hybridization studies” J. Am. Chem. Soc. 121:2921-2922; Marras et al. (1999) “Multiplex detection of single-nucleotide variation using molecular beacons” Genet. Anal. Biomol. Eng. 14:151-156; and Vet et al. (1999) “Multiplex detection of four pathogenic retroviruses using molecular beacons” Proc. Natl. Acad. Sci. U.S.A. 96:6394-6399. Additional details regarding MB construction and use is found in the patent literature, e.g., U.S. Pat. No. 5,925,517 (Jul. 20, 1999) to Tyagi et al. entitled “Detectably labeled dual conformation oligonucleotide probes, assays and kits;” U.S. Pat. No. 6,150,097 to Tyagi et al (Nov. 21, 2000) entitled “Nucleic acid detection probes having non-FRET fluorescence quenching and kits and assays including such probes” and U.S. Pat. No. 6,037,130 to Tyagi et al (Mar. 14, 2000), entitled “Wavelength-shifting probes and primers and their use in assays and kits.”

PCR detection and quantification using dual-labeled fluorogenic oligonucleotide probes, commonly referred to as “TaqMan™” probes, can also be performed according to the present invention. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. Accordingly, TaqMan™ probes are oligonucleotides that have a label and a quencher, where the label is released during amplification by the exonuclease action of the polymerase used in amplification. This provides a real time measure of amplification during synthesis. A variety of TaqMan™ reagents are commercially available, e.g., from Applied Biosystems (Division Headquarters in Foster City, Calif.) as well as from a variety of specialty vendors such as Biosearch Technologies (e.g., black hole quencher probes). Further details regarding dual-label probe strategies can be found, e.g., in WO092/02638.

Other similar methods include e.g. fluorescence resonance energy transfer between two adjacently hybridized probes, e.g., using the “LightCycler®” format described in U.S. Pat. No. 6,174,670.

Array-Based Marker Detection

Array-based detection can be performed using commercially available arrays, e.g., from Affymetrix (Santa Clara, Calif.) or other manufacturers. Reviews regarding the operation of nucleic acid arrays include Sapolsky et al. (1999) “High-throughput polymorphism screening and genotyping with high-density oligonucleotide arrays.” Genetic Analysis: Biomolecular Engineering 14:187-192; Lockhart (1998) “Mutant yeast on drugs” Nature Medicine 4:1235-1236; Fodor (1997) “Genes, Chips and the Human Genome.” FASEB Journal 11:A879; Fodor (1997) “Massively Parallel Genomics.” Science 277: 393-395; and Chee et al. (1996) “Accessing Genetic Information with High-Density DNA Arrays.” Science 274:610-614. Array based detection is a preferred method for identification markers of the invention in samples, due to the inherently high-throughput nature of array based detection.

A variety of probe arrays have been described in the literature and can be used in the context of the present invention for detection of markers that can be correlated to the phenotypes noted herein (treatment emergent weight gain, metabolic syndrome, obesity predisposition, insulin resistance, etc.). For example, DNA probe array chips or larger DNA probe array wafers (from which individual chips would otherwise be obtained by breaking up the wafer) are used in one embodiment of the invention. DNA probe array wafers generally comprise glass wafers on which high density arrays of DNA probes (short segments of DNA) have been placed. Each of these wafers can hold, for example, approximately 60 million DNA probes that are used to recognize longer sample DNA sequences (e.g., from individuals or populations, e.g., that comprise markers of interest). The recognition of sample DNA by the set of DNA probes on the glass wafer takes place through DNA hybridization. When a DNA sample hybridizes with an array of DNA probes, the sample binds to those probes that are complementary to the sample DNA sequence. By evaluating to which probes the sample DNA for an individual hybridizes more strongly, it is possible to determine whether a known sequence of nucleic acid is present or not in the sample, thereby determining whether a marker found in the nucleic acid is present. One can also use this approach to perform ASH, by controlling the hybridization conditions to permit single nucleotide discrimination, e.g., for SNP identification and for genotyping a sample for one or more SNPs.

The use of DNA probe arrays to obtain allele information typically involves the following general steps: design and manufacture of DNA probe arrays, preparation of the sample, hybridization of sample DNA to the array, detection of hybridization events and data analysis to determine sequence. Preferred wafers are manufactured using a process adapted from semiconductor manufacturing to achieve cost effectiveness and high quality, and are available, e.g., from Affymetrix, Inc of Santa Clara, Calif.

For example, probe arrays can be manufactured by light-directed chemical synthesis processes, which combine solid-phase chemical synthesis with photolithographic fabrication techniques as employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays can be synthesized simultaneously on a large glass wafer. This parallel process enhances reproducibility and helps achieve economies of scale.

Once fabricated, DNA probe arrays can be used to obtain data regarding presence and/or expression levels for markers of interest. The DNA samples may be tagged with biotin and/or a fluorescent reporter group by standard biochemical methods. The labeled samples are incubated with an array, and segments of the samples bind, or hybridize, with complementary sequences on the array. The array can be washed and/or stained to produce a hybridization pattern. The array is then scanned and the patterns of hybridization are detected by emission of light from the fluorescent reporter groups. Additional details regarding these procedures are found in the examples below. Because the identity and position of each probe on the array is known, the nature of the DNA sequences in the sample applied to the array can be determined. When these arrays are used for genotyping experiments, they can be referred to as genotyping arrays.

The nucleic acid sample to be analyzed is isolated, amplified and, typically, labeled with biotin and/or a fluorescent reporter group. The labeled nucleic acid sample is then incubated with the array using a fluidics station and hybridization oven. The array can be washed and or stained or counter-stained, as appropriate to the detection method. After hybridization, washing and staining, the array is inserted into a scanner, where patterns of hybridization are detected. The hybridization data are collected as light emitted from the fluorescent reporter groups already incorporated into the labeled nucleic acid, which is now bound to the probe array. Probes that most clearly match the labeled nucleic acid produce stronger signals than those that have mismatches. Since the sequence and position of each probe on the array are known, by complementarity, the identity of the nucleic acid sample applied to the probe array can be identified.

In one embodiment, two DNA samples may be differentially labeled and hybridized with a single set of the designed genotyping arrays. In this way two sets of data can be obtained from the same physical arrays. Labels that can be used include, but are not limited to, cychrome, fluorescein, or biotin (later stained with phycoerythrin-streptavidin after hybridization). Two-color labeling is described in U.S. Pat. No. 6,342,355, incorporated herein by reference in its entirety. Each array may be scanned such that the signal from both labels is detected simultaneously, or may be scanned twice to detect each signal separately.

Intensity data is collected by the scanner for all the markers for each of the individuals that are tested for presence of the marker. The measured intensities are a measure indicative of the amount of a particular marker present in the sample for a given individual (expression level and/or number of copies of the allele present in an individual, depending on whether genomic or expressed nucleic acids are analyzed). This can be used to determine whether the individual is homozygous or heterozygous for the marker of interest. The intensity data is processed to provide corresponding marker information for the various intensities.

Additional Details Regarding Amplified Variable Sequences, SSR, AFLP ASH, SNPs and Isozyme Markers

Amplified variable sequences refer to amplified sequences of the genome which exhibit high nucleic acid residue variability between members of the same species. All organisms have variable genomic sequences and each organism (with the exception of a clone) has a different set of variable sequences. Once identified, the presence of specific variable sequence can be used to predict phenotypic traits. Preferably, DNA from the genome serves as a template for amplification with primers that flank a variable sequence of DNA. The variable sequence is amplified and then sequenced.

Alternatively, self-sustained sequence replication can be used to identify genetic markers. Self-sustained sequence replication refers to a method of nucleic acid amplification using target nucleic acid sequences which are replicated exponentially, in vitro, under substantially isothermal conditions by using three enzymatic activities involved in retroviral replication: (1) reverse transcriptase, (2) Rnase H, and (3) a DNA-dependent RNA polymerase (Guatelli et al. (1990) Proc Natl Acad Sci USA 87:1874). By mimicking the retroviral strategy of RNA replication by means of cDNA intermediates, this reaction accumulates cDNA and RNA copies of the original target.

Amplified fragment length polymorphisms (AFLP) can also be used as genetic markers (Vos et al. (1995) Nucl Acids Res 23:4407). The phrase “amplified fragment length polymorphism” refers to selected restriction fragments which are amplified before or after cleavage by a restriction endonuclease. The amplification step allows easier detection of specific restriction fragments. AFLP allows the detection large numbers of polymorphic markers and has been used for genetic mapping (Becker et al. (1995) Mol Gen Genet 249:65; and Meksem et al. (1995) Mol Gen Genet 249:74).

Allele-specific hybridization (ASH) can be used to identify the genetic markers of the invention. ASH technology is based on the stable annealing of a short, single-stranded, oligonucleotide probe to a completely complementary single-strand target nucleic acid. Detection may be accomplished via an isotopic or non-isotopic label attached to the probe.

For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization. In this manner, only one of the alternative probes will hybridize to a target sample that is homozygous or homogenous for an allele. Samples that are heterozygous or heterogeneous for two alleles will hybridize to both of two alternative probes.

ASH markers are used as dominant markers where the presence or absence of only one allele is determined from hybridization or lack of hybridization by only one probe. The alternative allele may be inferred from the lack of hybridization. ASH probe and target molecules are optionally RNA or DNA; the target molecules are any length of nucleotides beyond the sequence that is complementary to the probe; the probe is designed to hybridize with either strand of a DNA target; the probe ranges in size to conform to variously stringent hybridization conditions, etc.

PCR allows the target sequence for ASH to be amplified from low concentrations of nucleic acid in relatively small volumes. Otherwise, the target sequence from genomic DNA is digested with a restriction endonuclease and size separated by gel electrophoresis. Hybridizations typically occur with the target sequence bound to the surface of a membrane or, as described in U.S. Pat. No. 5,468,613, the ASH probe sequence may be bound to a membrane.

In one embodiment, ASH data are typically obtained by amplifying nucleic acid fragments (amplicons) from genomic DNA using PCR, transferring the amplicon target DNA to a membrane in a dot-blot format, hybridizing a labeled oligonucleotide probe to the amplicon target, and observing the hybridization dots by autoradiography.

Single nucleotide polymorphisms (SNP) are markers that consist of a shared sequence differentiated on the basis of a single nucleotide. Typically, this distinction is detected by differential migration patterns of an amplicon comprising the SNP on e.g., an acrylamide gel. However, alternative modes of detection, such as hybridization, e.g., ASH, or RFLP analysis are also appropriate.

Isozyme markers can be employed as genetic markers, e.g., to track isozyme markers linked to the markers herein. Isozymes are multiple forms of enzymes that differ from one another in their amino acid, and therefore their nucleic acid sequences. Some isozymes are multimeric enzymes contain slightly different subunits. Other isozymes are either multimeric or monomeric but have been cleaved from the proenzyme at different sites in the amino acid sequence. Isozymes can be characterized and analyzed at the protein level, or alternatively, isozymes which differ at the nucleic acid level can be determined. In such cases any of the nucleic acid based methods described herein can be used to analyze isozyme markers.

Additional Details Regarding Nucleic Acid Amplification

As noted, nucleic acid amplification techniques such as PCR and LCR are well known in the art and can be applied to the present invention to amplify and/or detect nucleic acids of interest, such as nucleic acids comprising marker loci. Examples of techniques sufficient to direct persons of skill through such in vitro methods, including the polymerase chain reaction (PCR), the ligase chain reaction (LCR), Qβ-replicase amplification and other RNA polymerase mediated techniques (e.g., NASBA), are found in the references noted above, e.g., Innis, Sambrook, Ausubel, and Berger. Additional details are found in Mullis et al. (1987) U.S. Pat. No. 4,683,202; Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of amplifying large nucleic acids by PCR, which is useful in the context of positional cloning, are further summarized in Cheng et al. (1994) Nature 369: 684, and the references therein, in which PCR amplicons of up to 40 kb are generated. Methods for long-range PCR are disclosed, for example, in U.S. patent application Ser. No. 10/042,406, filed Jan. 9, 2002, entitled “Algorithms for Selection of Primer Pairs”; U.S. patent application Ser. No. 10/236,480, filed Sep. 9, 2002, entitled “Methods for Amplification of Nucleic Acids”; and U.S. Pat. No. 6,740,510, issued May 25, 2004, entitled “Methods for Amplification of Nucleic Acids”. U.S. Ser. No. 10/341,832 (filed Jan. 14, 2003) also provides details regarding primer picking methods for performing short range PCR.

Detection of Protein Expression Products

Proteins such as PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 and others encoded by the genes noted in Appendix 1 are encoded by nucleic acids, including those comprising markers that are correlated to the phenotypes of interest herein. For a description of the basic paradigm of molecular biology, including the expression (transcription and/or translation) of DNA into RNA into protein, see, Alberts et al. (2002) Molecular Biology of the Cell, 4^{th} Edition Taylor and Francis, Inc., ISBN: 0815332181 (“Alberts”), and Lodish et al. (1999) Molecular Cell Biology 4^{th} Edition W H Freeman & Co, ISBN: 071673706X (“Lodish”). Accordingly, proteins corresponding to PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 or other genes in Appendix 1 can be detected as markers, e.g., by detecting different protein isotypes between individuals or populations, or by detecting a differential presence, absence or expression level of such a protein of interest (e.g., expression level of PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 or a gene product of Appendix 1).

A variety of protein detection methods are known and can be used to distinguish markers. In addition to the various references noted supra, a variety of protein manipulation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods. 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000).

“Proteomic” detection methods, which detect many proteins simultaneously have been described. These can include various multidimensional electrophoresis methods (e.g., 2-d gel electrophoresis), mass spectrometry based methods (e.g., SELDI, MALDI, electrospray, etc.), or surface plasmon reasonance methods. For example, in MALDI, a sample is usually mixed with an appropriate matrix, placed on the surface of a probe and examined by laser desorption/ionization. The technique of MALDI is well known in the art. See, e.g., U.S. Pat. No. 5,045,694 (Beavis et al.), U.S. Pat. No. 5,202,561 (Gleissmann et al.), and U.S. Pat. No. 6,111,251 (Hillenkamp). Similarly, for SELDI, a first aliquot is contacted with a solid support-bound (e.g., substrate-bound) adsorbent. A substrate is typically a probe (e.g., a biochip) that can be positioned in an interrogatable relationship with a gas phase ion spectrometer. SELDI is also a well known technique, and has been applied to diagnostic proteomics. See, e.g. Issaq et al. (2003) “SELDI-TOF MS for Diagnostic Proteomics” Analytical Chemistry 75: 149A-155A.

In general, the above methods can be used to detect different forms (alleles) of proteins and/or can be used to detect different expression levels of the proteins (which can be due to allelic differences) between individuals, families, lines, populations, etc. Differences in expression levels, when controlled for environmental factors, can be indicative of different alleles at a QTL for the gene of interest, even if the encoded differentially expressed proteins are themselves identical. This occurs, for example, where there are multiple allelic forms of a gene in non-coding regions, e.g., regions such as promoters or enhancers that control gene expression. Thus, detection of differential expression levels can be used as a method of detecting allelic differences.

In other aspect of the present invention, a gene comprising, in linkage disequilibrium with, or under the control of a nucleic acid associated with treatment emergent weight gain, metabolic syndrome, insulin resistance or obesity may exhibit differential allelic expression. “Differential allelic expression” as used herein refers to both qualitative and quantitative differences in the allelic expression of multiple alleles of a single gene present in a cell. As such, a gene displaying differential allelic expression may have one allele expressed at a different time or level as compared to a second allele in the same cell/tissue. For example, an allele associated with metabolic syndrome may be expressed at a higher or lower level than an allele that is not associated with metabolic syndrome, even though both are alleles of the same gene and are present in the same cell/tissue. Differential allelic expression and analysis methods are disclosed in detail in U.S. patent application Ser. No. 10/438,184, filed May 13, 2003 and U.S. patent application Ser. No. 10/845,316, filed May 12, 2004, both of which are entitled “Allele-specific expression patterns.” Detection of a differential allelic expression pattern of one or more nucleic acids, or fragments, derivatives, polymorphisms, variants or complements thereof, associated with susceptibility to treatment emergent weight gain, metabolic syndrome, insulin resistance, or obesity is a prognostic and diagnostic for susceptibility to metabolic syndrome, insulin resistance, or obesity, respectively; likewise, detection of a differential allelic expression pattern of one or more nucleic acids, or fragments, derivatives, polymorphisms, variants or complements thereof, associated with resistance to treatment emergent weight gain, metabolic syndrome, insulin resistance, or obesity is a prognostic and diagnostic for resistance to metabolic syndrome, insulin resistance, or obesity, respectively.

Additional Details Regarding Types of Markers Appropriate for Screening

The biological markers that are screened for correlation to the phenotypes herein can be any of those types of markers that can be detected by screening, e.g., genetic markers such as allelic variants of a genetic locus (e.g., as in SNPs), expression markers (e.g., presence or quantity of mRNAs and/or proteins), and/or the like.

The nucleic acid of interest to be amplified, transcribed, translated and/or detected in the methods of the invention can be essentially any nucleic acid, though nucleic acids derived from human sources are especially relevant to the detection of markers associated with disease diagnosis and clinical applications. The sequences for many nucleic acids and amino acids (from which nucleic acid sequences can be derived via reverse translation) are available, including for PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 and/or the genes or gene products of Appendix 1. Common sequence repositories for known nucleic acids include GenBank® EMBL, DDBJ and the NCBI. Other repositories can easily be identified by searching the internet. The nucleic acid to be amplified, transcribed, translated and/or detected can be an RNA (e.g., where amplification includes RT-PCR or LCR, the Van-Gelder Eberwine reaction or Ribo-SPIA) or DNA (e.g., amplified DNA, cDNA or genomic DNA), or even any analogue thereof (e.g., for detection of synthetic nucleic acids or analogues thereof, e.g., where the sample of interest includes or is used to derive or synthesize artificial nucleic acids). Any variation in a nucleic acid sequence or expression level between individuals or populations can be detected as a marker, e.g., a mutation, a polymorphism, a single nucleotide polymorphism (SNP), an allele, an isotype, expression of an RNA or protein, etc. One can detect variation in sequence, expression levels or gene copy numbers as markers that can be correlated to treatment emergent weight gain, metabolic syndrome, obesity predisposition and/or insulin resistance.

For example, the methods of the invention are useful in screening samples derived from patients for a marker nucleic acid of interest, e.g., from bodily fluids (blood, saliva, urine etc.), tissue, and/or waste from the patient. Thus, stool, sputum, saliva, blood, lymph, tears, sweat, urine, vaginal secretions, ejaculatory fluid or the like can easily be screened for nucleic acids by the methods of the invention, as can essentially any tissue of interest that contains the appropriate nucleic acids. These samples are typically taken, following informed consent, from a patient by standard medical laboratory methods.

Prior to amplification and/or detection of a nucleic acid comprising a marker, the nucleic acid is optionally purified from the samples by any available method, e.g., those taught in Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2001 (“Sambrook”); and/or Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)). A plethora of kits are also commercially available for the purification of nucleic acids from cells or other samples (see, e.g., EasyPrep™, FlexiPrep™, both from Pharmacia Biotech; StrataClean™, from Stratagene; and, QIAprep™ from Qiagen). Alternately, samples can simply be directly subjected to amplification or detection, e.g., following aliquotting and/or dilution.

Examples of markers can include polymorphisms, single nucleotide polymorphisms, presence of one or more nucleic acids in a sample, absence of one or more nucleic acids in a sample, presence of one or more genomic DNA sequences, absence or one or more genomic DNA sequences, presence of one or more mRNAs, absence of one or more mRNAs, expression levels of one or more mRNAs, presence of one or more proteins, expression levels of one or more proteins, and/or data derived from any of the preceding or combinations thereof. Essentially any number of markers can be detected, using available methods, e.g., using array technologies that provide high density, high throughput marker mapping. Thus, at least about 10, 100, 1,000, 10,000, or even 100,000 or more genetic markers can be tested, simultaneously or in a serial fashion (or combination thereof), for correlation to a relevant phenotype, in the first and/or second population. Combinations of markers can also be desirably tested, e.g., to identify genetic combinations or combinations of expression patterns in populations that are correlated to the phenotype.

As noted, the biological marker to be detected can be any detectable biological component. Commonly detected markers include genetic markers (e.g., DNA sequence markers present in genomic DNA or expression products thereof) and expression markers (which can reflect genetically coded factors, environmental factors, or both). Where the markers are expression markers, the methods can include determining a first expression profile for a first individual or population (e.g., of one or more expressed markers, e.g., a set of expressed markers) and comparing the first expression profile to a second expression profile for the second individual or population. In this example, correlating expression marker(s) to a particular phenotype can include correlating the first or second expression profile to the phenotype of interest.

Probe/Primer Synthesis Methods

In general, synthetic methods for making oligonucleotides, including probes, primers, molecular beacons, PNAs, LNAs (locked nucleic acids), etc., are well known. For example, oligonucleotides can be synthesized chemically according to the solid phase phosphoramidite triester method described by Beaucage and Caruthers (1981), Tetrahedron Letts., 22(20):1859-1862, e.g., using a commercially available automated synthesizer, e.g., as described in Needham-VanDevanter et al. (1984) Nucleic Acids Res., 12:6159-6168. Oligonucleotides, including modified oligonucleotides can also be ordered from a variety of commercial sources known to persons of skill. There are many commercial providers of oligo synthesis services, and thus this is a broadly accessible technology. Any nucleic acid can be custom ordered from any of a variety of commercial sources, such as The Midland Certified Reagent Company (mcrc@oligos.com), The Great American Gene Company (www.genco.com), ExpressGen Inc. (www.expressgen.com), Operon Technologies Inc. (Alameda, Calif.) and many others. Similarly, PNAs can be custom ordered from any of a variety of sources, such as PeptidoGenic (pkim@ccnet.com), HTI Bio-products, inc. (htibio.com), BMA Biomedicals Ltd (U.K.), Bio•Synthesis, Inc., and many others.

In Silico Marker Detection

In some embodiments, in silico methods can be used to detect the marker loci of interest. For example, the sequence of a nucleic acid comprising the marker locus of interest can be stored in a computer. The desired marker locus sequence or its homolog can be identified using an appropriate nucleic acid search algorithm as provided by, for example, in such readily available programs as BLAST, or even simple word processors. The entire human genome has been sequenced and, thus, sequence information can be used to identify marker regions, flanking nucleic acids, etc.

Amplification Primers for Marker Detection

In some preferred embodiments, the molecular markers of the invention are detected using a suitable PCR-based detection method, where the size or sequence of the PCR amplicon is indicative of the absence or presence of the marker (e.g., a particular marker allele). In these types of methods, PCR primers are hybridized to the conserved regions flanking the polymorphic marker region.

It will be appreciated that, although many specific examples of primers are provided herein (see, Appendix 1), suitable primers to be used with the invention can be designed using any suitable method. It is not intended that the invention be limited to any particular primer or primer pair. For example, primers can be designed using any suitable software program, such as LASERGENE®, e.g., taking account of publicly available sequence information.

In some embodiments, the primers of the invention are radiolabelled, or labeled by any suitable means (e.g., using a non-radioactive fluorescent tag), to allow for rapid visualization of the different size amplicons following an amplification reaction without any additional labeling step or visualization step. In some embodiments, the primers are not labeled, and the amplicons are visualized following their size resolution, e.g., following agarose or acrylamide gel electrophoresis. In some embodiments, ethidium bromide staining of the PCR amplicons following size resolution allows visualization of the different size amplicons.

It is not intended that the primers of the invention be limited to generating an amplicon of any particular size. For example, the primers used to amplify the marker loci and alleles herein are not limited to amplifying the entire region of the relevant locus. The primers can generate an amplicon of any suitable length that is longer or shorter than those given as example amplicons in Appendix 1. In some embodiments, marker amplification produces an amplicon at least 20 nucleotides in length, or alternatively, at least 50 nucleotides in length, or alternatively, at least 100 nucleotides in length, or alternatively, at least 200 nucleotides in length.

Detection of Markers for Positional Cloning

In some embodiments, a nucleic acid probe is used to detect a nucleic acid that comprises a marker sequence. Such probes can be used, for example, in positional cloning to isolate nucleotide sequences linked to the marker nucleotide sequence. It is not intended that the nucleic acid probes of the invention be limited to any particular size. In some embodiments, nucleic acid probe is at least 20 nucleotides in length, or alternatively, at least 50 nucleotides in length, or alternatively, at least 100 nucleotides in length, or alternatively, at least 200 nucleotides in length.

A hybridized probe is detected using, autoradiography, fluorography or other similar detection techniques depending on the label to be detected. Examples of specific hybridization protocols are widely available in the art, see, e.g., Berger, Sambrook, and Ausubel, all herein.

Generation of Transgenic Cells and Organisms

The present invention also provides cells and organisms which are transformed with nucleic acids corresponding to QTL identified according to the invention. For example, such nucleic acids include chromosome intervals (e.g., genomic fragments), ORFs and/or cDNAs that encode genes that correspond or are linked to QTL for treatment emergent weight gain, metabolic syndrome, insulin resistance, and/or obesity predisposition. Additionally, the invention provides for the production of polypeptides that influence obesity, insulin resistance treatment emergent weight gain, and metabolic syndrome. This is useful, e.g., to influence treatment emergent weight gain, metabolic syndrome, obesity predisposition or insulin resistance in livestock populations. The generation of transgenic cells also provides commercially useful cells having defined genes that influence phenotype, thereby providing a platform for screening potential modulators of phenotype, as well as basic research into the mechanism of action for each of the genes of interest. In addition, gene therapy can be used to introduce desirable genes into individuals or populations thereof. Such gene therapies may be used to provide a treatment for a disorder exhibited by an individual, or may be used as a preventative measure to prevent the development of such a disorder in an individual at risk. Knock-out animals, such as knock-out mice, can be produced for any of the genes noted herein, to further identify phenotypic effects of the genes. Similarly, recombinant mice or other animals can be used as models for human disease, e.g., by knocking out any natural gene herein and introduction (e.g., via homologous recombination) of the human (or other species) gene into the animal. The effects of modulators on the heterologous human genes and gene products can then be monitored in the resulting in vivo model animal system.

General texts which describe molecular biological techniques for the cloning and manipulation of nucleic acids and production of encoded polypeptides include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.). Vol. 1-3, Cold Spring Harbor Laboratory, Cold Spring Harbor, N.Y., 2001 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2004 or later) (“Ausubel”)). These texts describe mutagenesis, the use of vectors, promoters and many other relevant topics related to, e.g., the generation of clones that comprise nucleic acids of interest, e.g., genes, marker loci, marker probes, QTL that segregate with marker loci, etc.

Host cells are genetically engineered (e.g., transduced, transfected, transformed, etc.) with the vectors of this invention (e.g., vectors, such as expression vectors which comprise an ORF derived from or related to a QTL) which can be, for example, a cloning vector, a shuttle vector or an expression vector. Such vectors are, for example, in the form of a plasmid, a phagemid, an agrobacterium, a virus, a naked polynucleotide (linear or circular), or a conjugated polynucleotide. Vectors can be introduced into bacteria, especially for the purpose of propagation and expansion. Additional details regarding nucleic acid introduction methods are found in Sambrook, Berger and Ausubel, infra. The method of introducing a nucleic acid of the present invention into a host cell is not critical to the instant invention, and it is not intended that the invention be limited to any particular method for introducing exogenous genetic material into a host cell. Thus, any suitable method, e.g., including but not limited to the methods provided herein, which provides for effective introduction of a nucleic acid into a cell or protoplast can be employed and finds use with the invention.

The engineered host cells can be cultured in conventional nutrient media modified as appropriate for such activities as, for example, activating promoters or selecting transformants. In addition to Sambrook, Berger and Ausubel, all infra, Atlas and Parks (eds) The Handbook of Microbiological Media (1993) CRC Press, Boca Raton, Fla. and available commercial literature such as the Life Science Research Cell Culture Catalogue (2004) from Sigma-Aldrich, Inc (St Louis, Mo.) (“Sigma-LSRCCC”) provide additional details.

Making Knock-Out Animals and Transgenics

Transgenic animals are a useful tool for studying gene function and testing putative gene or gene product modulators. Human (or other selected species) genes herein can be introduced in place of endogenous genes of a laboratory animal, making it possible to study function of the human (or other, e.g., livestock) gene or gene product in the easily manipulated and studied laboratory animal.

It will be appreciated that there is not always a precise correspondence for responses to modulators between homologous gene in different animals, making the ability to study the human or other species of interest (e.g., a livestock species) in a laboratory animal particularly useful. Although similar genetic manipulations can be performed in tissue culture, the interaction of genes and gene products in the context of an intact organism provides a more complete and physiologically relevant picture of such genes and gene products than can be achieved in simple cell-based screening assays. Accordingly, one feature of the invention is the creation of transgenic animals comprising heterologous genes of interest, e.g., a heterologous (PAPPA), peptidylglycine alpha amidating monooxygenase (PAM), pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, and/or HSF2.

In general, such a transgenic animal is simply an animal that has had appropriate genes (or partial genes, e.g., comprising coding sequences coupled to a promoter) introduced into one or more of its cells artificially. This is most commonly done in one of two ways. First, a DNA can be integrated randomly by injecting it into the pronucleus of a fertilized ovum. In this case, the DNA can integrate anywhere in the genome. In this approach, there is no need for homology between the injected DNA and the host genome. Second, targeted insertion can be accomplished by introducing the (heterologous) DNA into embryonic stem (ES) cells and selecting for cells in which the heterologous DNA has undergone homologous recombination with homologous sequences of the cellular genome. Typically, there are several kilobases of homology between the heterologous and genomic DNA, and positive selectable markers (e.g., antibiotic resistance genes) are included in the heterologous DNA to provide for selection of transformants. In addition, negative selectable markers (e.g., “toxic” genes such as barnase) can be used to select against cells that have incorporated DNA by non-homologous recombination (random insertion).

One common use of targeted insertion of DNA is to make knock-out mice. Typically, homologous recombination is used to insert a selectable gene driven by a constitutive promoter into an essential exon of the gene that one wishes to disrupt (e.g., the first coding exon). To accomplish this, the selectable marker is flanked by large stretches of DNA that match the genomic sequences surrounding the desired insertion point. Once this construct is electroporated into ES cells, the cells' own machinery performs the homologous recombination. To make it possible to select against ES cells that incorporate DNA by non-homologous recombination, it is common for targeting constructs to include a negatively selectable gene outside the region intended to undergo recombination (typically the gene is cloned adjacent to the shorter of the two regions of genomic homology). Because DNA lying outside the regions of genomic homology is lost during homologous recombination, cells undergoing homologous recombination cannot be selected against, whereas cells undergoing random integration of DNA often can. A commonly used gene for negative selection is the herpes virus thymidine kinase gene, which confers sensitivity to the drug gancyclovir.

Following positive selection and negative selection if desired, ES cell clones are screened for incorporation of the construct into the correct genomic locus. Typically, one designs a targeting construct so that a band normally seen on a Southern blot or following PCR amplification becomes replaced by a band of a predicted size when homologous recombination occurs. Since ES cells are diploid, only one allele is usually altered by the recombination event so, when appropriate targeting has occurred, one usually sees bands representing both wild type and targeted alleles.

The embryonic stem (ES) cells that are used for targeted insertion are derived from the inner cell masses of blastocysts (early mouse embryos). These cells are pluripotent, meaning they can develop into any type of tissue.

Once positive ES clones have been grown up and frozen, the production of transgenic animals can begin. Donor females are mated, blastocysts are harvested, and several ES cells are injected into each blastocyst. Blastocysts are then implanted into a uterine horn of each recipient. By choosing an appropriate donor strain, the detection of chimeric offspring (i.e., those in which some fraction of tissue is derived from the transgenic ES cells) can be as simple as observing hair and/or eye color. If the transgenic ES cells do not contribute to the germline (sperm or eggs), the transgene cannot be passed on to offspring.

Correlating Markers to Phenotypes

One aspect of the invention is a description of correlations between polymorphisms within or linked to the genes for PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 or others noted in Appendix 1 and treatment emergent weight gain, obesity predisposition, insulin resistance and metabolic syndrome phenotypes. An understanding of these correlations can be used in the present invention to correlate information regarding a set of polymorphisms that an individual or sample is determined to possess and a phenotype that they are likely to display. Further, higher order correlations that account for combinations of alleles in one or more different genes can also be assessed for correlations to phenotype.

These correlations can be performed by any method that can identify a relationship between an allele and a phenotype, or a combination of alleles and a combination of phenotypes. For example, alleles in one or more of PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 or other genes or loci in Appendix 1 can be correlated with one or more treatment emergent weight gain, obesity predisposition, insulin resistance and/or metabolic syndrome phenotypes. Most typically, these methods involve referencing a look up table that comprises correlations between alleles of the polymorphism and the phenotype. The table can include data for multiple allele-phenotype relationships and can take account of additive or other higher order effects of multiple allele-phenotype relationships, e.g., through the use of statistical tools such as principle component analysis, heuristic algorithms, etc

Correlation of a marker to a phenotype optionally includes performing one or more statistical tests for correlation. Many statistical tests are known, and most are computer-implemented for ease of analysis. A variety of statistical methods of determining associations/correlations between phenotypic traits and biological markers are known and can be applied to the present invention. For an introduction to the topic, see, Hartl (1981) A Primer of Population Genetics Washington University, Saint Louis Sinauer Associates, Inc. Sunderland, Mass. ISBN: 0-087893-271-2. A variety of appropriate statistical models are described in Lynch and Walsh (1998) Genetics and Analysis of Quantitative Traits, Sinauer Associates, Inc. Sunderland Mass. ISBN 0-87893-481-2. These models can, for example, provide for correlations between genotypic and phenotypic values, characterize the influence of a locus on a phenotype, sort out the relationship between environment and genotype, determine dominance or penetrance of genes, determine maternal and other epigenetic effects, determine principle components in an analysis (via principle component analysis, or “PCA”), and the like. The references cited in these texts provides considerable further detail on statistical models for correlating markers and phenotype.

In addition to standard statistical methods for determining correlation, other methods that determine correlations by pattern recognition and training, such as the use of genetic algorithms, can be used to determine correlations between markers and phenotypes. This is particularly useful when identifying higher order correlations between multiple alleles and multiple phenotypes. To illustrate, neural network approaches can be coupled to genetic algorithm-type programming for heuristic development of a structure-function data space model that determines correlations between genetic information and phenotypic outcomes. For example, NNUGA (Neural Network Using Genetic Algorithms) is an available program ( e.g., on the world wide web at cs.bgu.ac.il/˜omri/NNUGA which couples neural networks and genetic algorithms. An introduction to neural networks can be found, e.g., in Kevin Gurney, An Introduction to Neural Networks, UCL Press (1999) and on the world wide web at shef.ac.uk/psychology/gurney/notes/index.html. Additional useful neural network references include those noted above in regard to genetic algorithms and, e.g., Bishop, Neural Networks for Pattern Recognition, Oxford University Press (1995), and Ripley et al., Pattern Recognition and Neural Networks, Cambridge University Press (1995).

Additional references that are useful in understanding data analysis applications for using and establishing correlations, principle components of an analysis, neural network modeling and the like, include, e.g., Hinchliffe, Modeling Molecular Structures, John Wiley and Sons (1996), Gibas and Jambeck, Bioinformatics Computer Skills, O'Reilly (2001), Pevzner, Computational Molecular Biology and Algorithmic Approach, The MIT Press (2000), Durbin et al., Biological Sequence Analysis: Probabilistic Models of Proteins and Nucleic Acids, Cambridge University Press (1998), and Rashidi and Buehler, Bioinformatic Basics: Applications in Biological Science and Medicine, CRC Press LLC (2000).

In any case, essentially any statistical test can be applied in a computer implemented model, by standard programming methods, or using any of a variety of “off the shelf” software packages that perform such statistical analyses, including, for example, those noted above and those that are commercially available, e.g., from Partek Incorporated (St. Peters, Mo.; www.partek.com), e.g., that provide software for pattern recognition (e.g., which provide Partek Pro 2000 Pattern Recognition Software) which can be applied to genetic algorithms for multivariate data analysis, interactive visualization, variable selection, neural network & statistical modeling, etc. Relationships can be analyzed, e.g., by Principal Components Analysis (PCA) mapped mapped scatterplots and biplots, Multi-Dimensional Scaling (MDS) Multi-Dimensional Scaling (MDS) mapped scatterplots, star plots, etc. Available software for performing correlation analysis includes SAS, R and MathLab.

In any case, the marker(s), whether polymorphisms or expression patterns, can be used for any of a variety of genetic analyses. For example, once markers have been identified, as in the present case, they can be used in a number of different assays for association studies. For example, probes can be designed for microarrays that interrogate these markers. Other exemplary assays include, e.g., the Taqman assays and molecular beacon assays described supra, as well as conventional PCR and/or sequencing techniques.

Additional details regarding association studies can be found in Ser. No. 10/106,097, filed Mar. 26, 2002, entitled “Methods for Genomic Analysis;” Ser. No. 10/042,819, filed Jan. 7, 2002, entitled “Genetic Analysis Systems and Methods;” Ser. No. 10/286,417, filed Oct. 31, 2002, entitled “Methods for Genomic Analysis;” Ser. No. 10/768,788, filed Jan. 30, 2004, entitled “Apparatus and Methods for Analyzing and Characterizing Nucleic Acid Sequences;” Ser. No. 10/447,685, filed May 28, 2003, entitled “Liver Related Disease Compositions and Methods;” Ser. No. 10/970,761, filed Oct. 20, 2004, entitled “Improved Analysis Methods and Apparatus for Individual Genotyping” (methods for individual genotyping); Ser. No. 10/956,224, filed Sep. 30, 2004, entitled “Methods for Genetic Analysis.”

In some embodiments, the marker data is used to perform association studies to show correlations between markers and phenotypes. This can be accomplished by determining marker characteristics in individuals with the phenotype of interest (i.e., individuals or populations displaying the phenotype of interest) and comparing the allele frequency or other characteristics (expression levels, etc.) of the markers in these individuals to the allele frequency or other characteristics in a control group of individuals. Such marker determinations can be conducted on a genome-wide basis, or can be focused on specific regions of the genome (e.g., haplotype blocks of interest). In one embodiment, markers that are linked to the genes for PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2, and/or other genes or loci in Appendix 1 are assessed for correlation to one or more specific phenotypes.

In addition to the other embodiments of the methods of the present invention disclosed herein, the methods additionally allow for the “dissection” of a phenotype. That is, a particular phenotypes can result from two or more different genetic bases. For example, treatment emergent weight gain, obesity, insulin resistance or metabolic syndrome susceptibility phenotype in one individual may be the result of a “defect” (or simply a particular allele—“defect” with respect to a susceptibility phenotype is context dependent, e.g., whether the phenotype is desirable or undesirable in the individual in a given environment) in a gene for PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 and/or others in Appendix 1, while the same basic phenotype in a different individual may be the result of multiple “defects” in PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 and/or others in Appendix 1. Thus, scanning a plurality of markers (e.g., as in genome or haplotype block scanning) allows for the dissection of varying genetic bases for similar (or graduated) phenotypes.

As described in the previous paragraph, one method of conducting association studies is to compare the allele frequency (or expression level) of markers in individuals with a phenotype of interest (“case group”) to the allele frequency in a control group of individuals. In one method, informative SNPs are used to make the SNP haplotype pattern comparison (an “informative SNP” is genetic SNP marker such as a SNP or subset (more than one) of SNPs in a genome or haplotype block that tends to distinguish one SNP or genome or haplotype pattern from other SNPs, genomes or haplotype patterns). The approach of using informative SNPs has an advantage over other whole genome scanning or genotyping methods known in the art, for instead of reading all 3 billion bases of each individual's genome-or even reading the 3-4 million common SNPs that may be found—only informative SNPs from a sample population need to be detected. Reading these particular, informative SNPs provides sufficient information to allow statistically accurate association data to be extracted from specific experimental populations, as described above.

Thus, in an embodiment of one method of determining genetic associations, the allele frequency of informative SNPs is determined for genomes of a control population that do not display the phenotype. The allele frequency of informative SNPs is also determined for genomes of a population that do display the phenotype. The informative SNP allele frequencies are compared. Allele frequency comparisons can be made, for example, by determining the allele frequency (number of instances of a particular allele in a population divided by the total number of alleles) at each informative SNP location in each population and comparing these allele frequencies. The informative SNPs displaying a difference between the allele frequency of occurrence in the control versus case populations/groups are selected for analysis. Once informative SNPs are selected, the SNP haplotype block(s) that contain the informative SNPs are identified, which in turn identifies a genomic region of interest that is correlated with the phenotype. The genomic regions can be analyzed by genetic or any biological methods known in the art e.g., for use as drug discovery targets or as diagnostic markers.

Systems for Identifying a Treatment Emergent Weight Gain, Metabolic Syndrome Phenotype, and Insulin Resistance Phenotype, or an Obesity Predisposition Phenotype

Systems for performing the above correlations are also a feature of the invention. Typically, the system will include system instructions that correlate the presence or absence of an allele (whether detected directly or, e.g., through expression levels) with a predicted treatment emergent weight gain phenotype, metabolic syndrome phenotype, insulin resistance phenotype, or obesity predisposition phenotype. The system instructions can compare detected information as to allele sequence or expression level with a database that includes correlations between the alleles and the relevant phenotypes. As noted above, this database can be multidimensional, thereby including higher-order relationships between combinations of alleles and the relevant phenotypes. These relationships can be stored in any number of look-up tables, e.g., taking the form of spreadsheets (e.g., Excel™ spreadsheets) or databases such as an Access™, SQL™, Oracle™, Paradox™, or similar database. The system includes provisions for inputting sample-specific information regarding allele detection information, e.g., through an automated or user interface and for comparing that information to the look up tables.

Optionally, the system instructions can also include software that accepts diagnostic information associated with any detected allele information, e.g., a diagnosis that a subject with the relevant allele has a particular phenotype (treatment emergent weight gain, metabolic syndrome, obesity predisposition, insulin resistance). This software can be heuristic in nature, using such inputted associations to improve the accuracy of the look up tables and/or interpretation of the look up tables by the system. A variety of such approaches, including neural networks, Markov modeling, and other statistical analysis are described above.

The invention provides data acquisition modules for detecting one or more detectable genetic marker(s) (e.g., one or more array comprising one or more biomolecular probes, detectors, fluid handlers, or the like). The biomolecular probes of such a data acquisition module can include any that are appropriate for detecting the biological marker, e.g., oligonucleotide probes, proteins, aptamers, antibodies, etc. These can include sample handlers (e.g., fluid handlers), robotics, microfluidic systems, nucleic acid or protein purification modules, arrays (e.g., nucleic acid arrays), detectors, thermocyclers or combinations thereof, e.g., for acquiring samples, diluting or aliquoting samples, purifying marker materials (e.g., nucleic acids or proteins), amplifying marker nucleic acids, detecting amplified marker nucleic acids, and the like.

For example, automated devices that can be incorporated into the systems herein have been used to assess a variety of biological phenomena, including, e.g., expression levels of genes in response to selected stimuli (Service (1998) “Microchips Arrays Put DNA on the Spot” Science 282:396-399), high throughput DNA genotyping (Zhang et al. (1999) “Automated and Integrated System for High-Throughput DNA Genotyping Directly from Blood” Anal. Chem. 71:1138-1145) and many others. Similarly, integrated systems for performing mixing experiments, DNA amplification, DNA sequencing and the like are also available. See, e.g., Service (1998) “Coming Soon: the Pocket DNA Sequencer” Science 282: 399-401. A variety of automated system components are available, e.g., from Caliper Technologies (Hopkinton, Mass.), which utilize various Zymate systems, which typically include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif.). Similarly, commercially available microfluidic systems that can be used as system components in the present invention include those from Agilent technologies and the Caliper Technologies. Furthermore, the patent and technical literature includes numerous examples of microfluidic systems, including those that can interface directly with microwell plates for automated fluid handling.

Any of a variety of liquid handling and/or array configurations can be used in the systems herein. One common format for use in the systems herein is a microtiter plate, in which the array or liquid handler includes a microtiter tray. Such trays are commercially available and can be ordered in a variety of well sizes and numbers of wells per tray, as well as with any of a variety of functionalized surfaces for binding of assay or array components. Common trays include the ubiquitous 96 well plate, with 384 and 1536 well plates also in common use. Samples can be processed in such trays, with all of the processing steps being performed in the trays. Samples can also be processed in microfluidic apparatus, or combinations of microtiter and microfluidic apparatus.

In addition to liquid phase arrays, components can be stored in or analyzed on solid phase arrays. These arrays fix materials in a spatially accessible pattern (e.g., a grid of rows and columns) onto a solid substrate such as a membrane (e.g., nylon or nitrocellulose), a polymer or ceramic surface, a glass or modified silica surface, a metal surface, or the like. Components can be accessed, e.g., by hybridization, by local rehydration (e.g., using a pipette or other fluid handling element) and fluidic transfer, or by scraping the array or cutting out sites of interest on the array.

The system can also include detection apparatus that is used to detect allele information, using any of the approached noted herein. For example, a detector configured to detect real-time PCR products (e.g., a light detector, such as a fluorescence detector) or an array reader can be incorporated into the system. For example, the detector can be configured to detect a light emission from a hybridization or amplification reaction comprising an allele of interest, wherein the light emission is indicative of the presence or absence of the allele. Optionally, an operable linkage between the detector and a computer that comprises the system instructions noted above is provided, allowing for automatic input of detected allele-specific information to the computer, which can, e.g., store the database information and/or execute the system instructions to compare the detected allele specific information to the look up table.

Probes that are used to generate information detected by the detector can also be incorporated within the system, along with any other hardware or software for using the probes to detect the amplicon. These can include thermocycler elements (e.g., for performing PCR or LCR amplification of the allele to be detected by the probes), arrays upon which the probes are arrayed and/or hybridized, or the like. The fluid handling elements noted above for processing samples, can be used for moving sample materials (e.g., template nucleic acids and/or proteins to be detected) primers, probes, amplicons, or the like into contact with one another. For example, the system can include a set of marker probes or primers configured to detect at least one allele of one or more genes or linked loci associated with treatment emergent weight gain, metabolic syndrome, obesity predisposition or insulin resistance phenotype, where the gene encodes PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 and/or others in Appendix 1. The detector module is configured to detect one or more signal outputs from the set of marker probes or primers, or an amplicon produced from the set of marker probes or primers, thereby identifying the presence or absence of the allele.

The sample to be analyzed is optionally part of the system, or can be considered separate from it. The sample optionally includes e.g., genomic DNA, amplified genomic DNA, cDNA, amplified cDNA, RNA, amplified RNA, proteins, etc., as noted herein. In one aspect, the sample is derived from a mammal such as a human patient.

Optionally, system components for interfacing with a user are provided. For example, the systems can include a user viewable display for viewing an output of computer-implemented system instructions, user input devices (e.g., keyboards or pointing devices such as a mouse) for inputting user commands and activating the system, etc. Typically, the system of interest includes a computer, wherein the various computer-implemented system instructions are embodied in computer software, e.g., stored on computer readable media.

Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Sequel™, Oracle™, Paradox™) can be adapted to the present invention by inputting a character string corresponding to an allele herein, or an association between an allele and a phenotype. For example, the systems can include software having the appropriate character string information, e.g., used in conjunction with a user interface (e.g., a GUI in a standard operating system such as a Windows, Macintosh or LINUX system) to manipulate strings of characters. Specialized sequence alignment programs such as BLAST can also be incorporated into the systems of the invention for alignment of nucleic acids or proteins (or corresponding character strings) e.g., for identifying and relating alleles.

As noted, systems can include a computer with an appropriate database and an allele sequence or correlation of the invention. Software for aligning sequences, as well as data sets entered into the software system comprising any of the sequences herein can be a feature of the invention. The computer can be, e.g., a PC (Intel x86 or Pentium chip-compatible DOS™, OS2™ WINDOWS™ WINDOWS NT™, WINDOWS95™, WINDOWS98™, WINDOWS2000, WINDOWSME, or LINUX based machine, a MACINTOSH™, Power PC, or a UNIX based (e.g., SUN™ work station or LINUX based machine) or other commercially common computer which is known to one of skill. Software for entering and aligning or otherwise manipulating sequences is available, e.g., BLASTP and BLASTN, or can easily be constructed by one of skill using a standard programming language such as Visualbasic, Fortran, Basic, Java, or the like.

Methods of Identifying Modulators of Treatment Emergent Weight Gain, Metabolic Syndrome, Insulin Resistance, or Obesity Predisposition

In addition to providing various diagnostic and prognostic markers for identifying metabolic syndrome, etc., the invention also provides methods of identifying modulators of treatment emergent weight gain, a metabolic syndrome phenotype, an insulin resistance phenotype, or an obesity predisposition phenotype. In the methods, a potential modulator is contacted to a relevant protein (PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 or others for the genes or loci in Appendix 1) or to a nucleic acid that encodes such a protein. An effect of the potential modulator on the gene or gene product is detected, thereby identifying whether the potential modulator modulates the underlying molecular basis for the treatment emergent weight gain, metabolic syndrome phenotype, the insulin resistance phenotype, or the obesity predisposition phenotype.

In addition, the methods can include, e.g., administering one or more putative modulator to an individual that displays a relevant phenotype and determining whether the putative modulator modulates the phenotype in the individual, e.g., in the context of a clinical trial or treatment. This, in turn, determines whether the putative modulator is clinically useful.

The gene or gene product that is contacted by the modulator can include any allelic form noted herein. Allelic forms, whether genes or proteins, that positively correlate to undesirable treatment emergent weight gain, metabolic syndrome, obesity or insulin resistance phenotypes are preferred targets for modulator screening.

Effects of interest that can be screened for include: (a) increased or decreased expression of PAPPA, PAM, pf20, DNAH11, PKD1, KCNMA1, PKHD1, NRXN3, EPHA7, ROS1, FKSG87, C3orf6, TOX, DLG2, MDS 1, FABP2, EFA6R, FLJ20125, C1orf10, CHL1, BICD1, KREMEN1, ADARB2, A2BP1, MGC4309, PIGR, PCSK7, HSF2 or other gene products in Appendix 1 in the presence of the modulator; (b) a change in the timing or location of expression of PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 and/or others in Appendix 1 in the presence of the modulator; (c) a change in localization of the proteins encoded by PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 and/or others in the presence of the modulator; (d) an increased or decreased cleavage of IGFBP4 by PAPPA in the presence of the modulator; (e) an increased or decreased catalysis of peptide cleavage by PAM in the presence of the modulator; (f) a change in function of cilia comprising pf20and/or DNAH11 in the presence of the modulator; (g) a change in association (affinity, etc.) of PKD1 gene product, e.g., polycystin-1, with PKD2 gene product, e.g., polycystin-2 in the presence of the modulator; (h) a change in localization of polycystin-2 in or to a plasma membrane in the presence of the modulator; (i) a change in activity of a channel comprising a polycystin-1 in the presence of the modulator; (j) a change in localization of a KCNMA1 gene product in the presence of the modulator; and (k) a change in activity of a channel comprising a KCNMA1 gene product in the presence of the modulator.

The precise format of the modulator screen will, of course, vary, depending on the effect(s) being detected and the equipment available. Northern analysis, quantitative RT-PCR and/or array-based detection formats can be used to distinguish expression levels of genes noted above. Protein expression levels can also be detected using available methods, such as western blotting, ELISA analysis, antibody hybridization, BIAcore, or the like. Any of these methods can be used to distinguish changes in expression levels of PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2, or others in Appendix 1, that result from a potential modulator.

Accordingly, one may screen for potential modulators of PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 or others in Appendix 1 for activity or expression. For example, potential modulators (small molecules, organic molecules, inorganic molecules, proteins, hormones, transcription factors, or the like) can be contacted to a cell comprising an allele of interest and an effect on activity or expression (or both) of PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 or others of Appendix 1 can be detected. For example, expression of PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, and/or HSF2 can be detected, e.g., via northern analysis or quantitative (optionally real time) RT-PCR, before and after application of potential expression modulators. Similarly, promoter regions of the various genes (e.g., generally sequences in the region of the start site of transcription, e.g., within 5 KB of the start site, e.g., 1 KB, or less e.g., within 500 BP or 250 BP or 100 BP of the start site) can be coupled to reporter constructs (CAT, beta-galactosidase, luciferase or any other available reporter) and can be similarly be tested for expression activity modulation by the potential modulator. In either case, the assays can be performed in a high-throughput fashion, e.g., using automated fluid handling and/or detection systems, in serial or parallel fashion. Similarly, activity modulators can be tested by contacting a potential modulator to an appropriate cell using any of the activity detection methods herein, regardless of whether the activity that is detected is the result of activity modulation, expression modulation or both. These assays can be in vitro, cell-based, or can be screens for modulator activity performed on laboratory animals such as knock-out transgenic mice comprising a gene of interest.

Biosensors for detecting modulator activity detection are also a feature of the invention. These include devices or systems that comprise PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 or others of Appendix 1 coupled to a readout that measures or displays one or more activity of the protein. Thus, any of the above described assay components can be configured as a biosensor by operably coupling the appropriate assay components to a readout. The readout can be optical (e.g., to detect cell markers or cell survival) electrical (e.g., coupled to a FET, a BIAcore, or any of a variety of others), spectrographic, or the like, and can optionally include a user-viewable display (e.g., a CRT or optical viewing station). The biosensor can be coupled to robotics or other automation, e.g., microfluidic systems, that direct contact of the putative modulators to the proteins of the invention, e.g., for automated high-throughput analysis of putative modulator activity. A large variety of automated systems that can be adapted to use with the biosensors of the invention are commercially available. For example, automated systems have been made to assess a variety of biological phenomena, including, e.g., expression levels of genes in response to selected stimuli (Service (1998) “Microchips Arrays Put DNA on the Spot” Science 282:396-399). Laboratory systems can also perform, e.g., repetitive fluid handling operations (e.g., pipetting) for transferring material to or from reagent storage systems that comprise arrays, such as microtiter trays or other chip trays, which are used as basic container elements for a variety of automated laboratory methods. Similarly, the systems manipulate, e.g., microtiter trays and control a variety of environmental conditions such as temperature, exposure to light or air, and the like. Many such automated systems are commercially available and are described herein, including those described above. These include various Zymate systems, ORCA® robots, microfluidic devices, etc. For example, the LabMicrofluidic device® high throughput screening system (HTS) by Caliper Technologies, Mountain View, Calif. can be adapted for use in the present invention to screen for modulator activity.

In general, methods and sensors for detecting protein expression level and activity are available, including those taught in the various references above, including R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and Satinder Ahuja ed., Handbook of Bioseparations, Academic Press (2000). “Proteomic” detection methods, which detect many proteins simultaneously have been described and are also noted above, including various multidimensional electrophoresis methods (e.g., 2-d gel electrophoresis), mass spectrometry based methods (e.g., SELDI, MALDI, electrospray, etc.), or surface plasmon reasonance methods. These can also be used to track protein activity and/or expression level.

Similarly, nucleic acid expression levels (e.g., mRNA) can be detected using any available method, including northern analysis, quantitative RT-PCR, or the like. References sufficient to guide one of skill through these methods are readily available, including Ausubel, Sambrook and Berger.

Whole animal assays can also be used to assess the effects of modulators on cells or whole animals (e.g., transgenic knock-out mice), e.g., by monitoring an effect on a cell-based phenomenon, a change in displayed animal phenotype, or the like.

Potential modulator libraries to be screened for effects on PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2, etc., expression and/or activity are available. These libraries can be random, or can be targeted.

Targeted libraries include those designed using any form of a rational design technique that selects scaffolds or building blocks to generate combinatorial libraries. These techniques include a number of methods for the design and combinatorial synthesis of target-focused libraries, including morphing with bioisosteric transformations, analysis of target-specific privileged structures, and the like. In general, where information regarding structure of PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2 or others of Appendix 1 is available, likely binding partners can be designed, e.g., using flexible docking approaches, or the like. Similarly, random libraries exist for a variety of basic chemical scaffolds. In either case, many thousands of scaffolds and building blocks for chemical libraries are available, including those with polypeptide, nucleic acid, carbohydrate, and other backbones. Commercially available libraries and library design services include those offered by Chemical Diversity (San Diego, Calif.), Affymetrix (Santa Clara, Calif.), Sigma (St. Louis Mo.), ChemBridge Research Laboratories (San Diego, Calif.), TimTec (Newark, Del.), Nuevolution A/S (Copenhagen, Denmark) and many others.

Kits for treatment of a treatment emergent weight gain, metabolic syndrome, obesity predisposition or insulin resistance phenotype can include a modulator identified as noted above and instructions for administering the compound to a patient to treat treatment emergent weight gain, metabolic syndrome, obesity predisposition and/or insulin resistance.

Cell Rescue and Therapeutic Administration

In one aspect, the invention includes rescue of a cell that is defective in function of one or more endogenous genes or polypeptides for PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, HSF2, and/or others of Appendix 1 (thus conferring the relevant phenotype of interest, e.g., treatment emergent weight gain, metabolic syndrome, obesity, insulin resistance, etc.). This can be accomplished simply by introducing a new copy of the gene (or a heterologous nucleic acid that expresses the relevant protein), i.e., a gene having an allele that is desired, into the cell. Other approaches, such as homologous recombination to repair the defective gene (e.g., via chimeraplasty) can also be performed. In any event, rescue of function can be measured, e.g., in any of the assays noted herein. Indeed, this method can be used as a general method of screening cells in vitro for a PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, and/or HSF2 expression or activity (or expression or activity of any gene or gene product of Appendix 1). Accordingly, in vitro rescue of function is useful in this context for the myriad in vitro screening methods noted above. The cells that are rescued can include cells in culture, (including primary or secondary cell culture from patients, as well as cultures of well-established cells). Where the cells are isolated from a patient, this has additional diagnostic utility in establishing which PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, and/or HSF2 or other Appendix 1 sequence is defective in a patient that presents with a relevant phenotype.

In another aspect, the cell rescue occurs in a patient, e.g., a human or veterinary patient, e.g., to remedy a metabolic defect. Thus, one aspect of the invention is gene therapy to remedy metabolic defects (or even simply to enhance metabolic phenotypes), in human or veterinary applications. In these applications, the nucleic acids of the invention are optionally cloned into appropriate gene therapy vectors (and/or are simply delivered as naked or liposome-conjugated nucleic acids), which are then delivered, optionally in combination with appropriate carriers or delivery agents. Proteins can also be delivered directly, but delivery of the nucleic acid is typically preferred in applications where stable expression is desired. Similarly, modulators of any metabolic defect that are identified by the methods herein can be used therapeutically.

Compositions for administration, e.g., comprise a therapeutically effective amount of the modulator, gene therapy vector or other relevant nucleic acid, and a pharmaceutically acceptable carrier or excipient. Such a carrier or excipient includes, but is not limited to, saline, buffered saline, dextrose, water, glycerol, ethanol, and/or combinations thereof. The formulation is made to suit the mode of administration. In general, methods of administering gene therapy vectors for topical use are well known in the art and can be applied to administration of the nucleic acids of the invention.

Therapeutic compositions comprising one or more modulator or gene therapy nucleic acid of the invention are optionally tested in one or more appropriate in vitro and/or in vivo animal model of disease, to confirm efficacy, tissue metabolism, and to estimate dosages, according to methods well known in the art. In particular, dosages can initially be determined by activity, stability or other suitable measures of the formulation.

Administration is by any of the routes normally used for introducing a molecule into ultimate contact with cells. Modulators and/or nucleic acids that encode PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, and/or HSF2 and/or other Appendix 1 sequence can be administered in any suitable manner, optionally with one or more pharmaceutically acceptable carriers. Suitable methods of administering such nucleic acids in the context of the present invention to a patient are available, and, although more than one route can be used to administer a particular composition, a particular route can often provide a more immediate and more effective action or reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention. Compositions can be administered by a number of routes including, but not limited to: oral, intravenous, intraperitoneal, intramuscular, transdermal, subcutaneous, topical, sublingual, or rectal administration. Compositions can be administered via liposomes (e.g., topically), or via topical delivery of naked DNA or viral vectors. Such administration routes and appropriate formulations are generally known to those of skill in the art.

The compositions, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations of packaged nucleic acid can be presented in unit-dose or multi-dose sealed containers, such as ampules and vials.

The dose administered to a patient, in the context of the present invention, is sufficient to effect a beneficial therapeutic response in the patient over time. The dose is determined by the efficacy of the particular vector, or other formulation, and the activity, stability or serum half-life of the polypeptide which is expressed, and the condition of the patient, as well as the body weight or surface area of the patient to be treated. The size of the dose is also determined by the existence, nature, and extent of any adverse side-effects that accompany the administration of a particular vector, formulation, or the like in a particular patient. In determining the effective amount of the vector or formulation to be administered in the treatment of disease, the physician evaluates local expression, or circulating plasma levels, formulation toxicities, progression of the relevant disease, and/or where relevant, the production of antibodies to proteins encoded by the polynucleotides. The dose administered, e.g., to a 70 kilogram patient are typically in the range equivalent to dosages of currently-used therapeutic proteins, adjusted for the altered activity or serum half-life of the relevant composition. The vectors of this invention can supplement treatment conditions by any known conventional therapy.

For administration, formulations of the present invention are administered at a rate determined by the LD-50 of the relevant formulation, and/or observation of any side-effects of the vectors of the invention at various concentrations, e.g., as applied to the mass or topical delivery area and overall health of the patient. Administration can be accomplished via single or divided doses.

If a patient undergoing treatment develops fevers, chills, or muscle aches, he/she receives the appropriate dose of aspirin, ibuprofen, acetaminophen or other pain/fever controlling drug. Patients who experience reactions to the compositions, such as fever, muscle aches, and chills are premedicated 30 minutes prior to the future infusions with either aspirin, acetaminophen, or, e.g., diphenhydramine. Meperidine is used for more severe chills and muscle aches that do not quickly respond to antipyretics and antihistamines. Treatment is slowed or discontinued depending upon the severity of the reaction.

### EXAMPLES

The following examples illustrate, but do not limit the invention. One of skill will recognize a variety of non-critical parameters that can be modified to achieve essentially similar results.

### Example 1

The entire human genome was scanned to identify common polymorphisms using microarray technology platforms as described in U.S. Ser. No. 10/106,097, entitled “Methods for Genomic Analysis”, filed on Mar. 26, 2002, assigned to the same assignee as the present application; U.S. Ser. No. 10/284,444, entitled “Chromosome 21 SNPs, SNP Groups and SNP Patterns,” filed on Oct. 31, 2002, assigned to the same assignee as the present application; and Ser. No. 10/042,819, entitled “Whole Genome Scanning,” filed on Jan. 7, 2002, assigned to the same assignee as the present application, all of which are incorporated herein by reference. The microarrays are manufactured using a process adapted from semiconductor manufacturing to achieve cost effectiveness and high quality.

### Example 2

Polymorphisms identified in Example 1 were grouped into haplotype blocks and haplotype patterns using methods disclosed in U.S. Ser. No. 10/106,097, entitled “Methods for Genomic Analysis”, filed Mar. 26, 2002 (Attorney Docket 200/1005-10), incorporated herein by reference. Representative polymorphisms, haplotype blocks and haplotype patterns from an entire human chromosome (chromosome 21) are disclosed in, for example, Patil, N. et al, “Blocks of Limited Haplotype Diversity Revealed by High-Resolution Scanning of Human Chromosome 21” Science 294, 1719-1723 (2001) and the associated supplemental materials, incorporated herein by reference.

### Example 3

DNA from each individual in the case (obesity phenotype) and control (non-obese phenotype) groups was purified by methods well known in the art. The samples ranged between 2-10 milliliters each. The concentrations of each DNA sample were adjusted to create stock solutions with DNA concentrations between 0.4 μg/μl and 0.6 μg/μl.

To further evaluate the purified DNA, 0.1 microgram of DNA was analyzed by agarose gel electrophoresis on a 0.8% agarose gel containing 3-5 μl of 10 mg/ml ethidium bromide per 100 ml of agarose. 2 μl of the DNA stock solution were added to enough water to create a 0.05 μg/μl dilution. Standard loading buffer was added to the sample and the sample was loaded onto the gel. The gel was run at 150 volts for 40-45 minutes, and then subjected to ultraviolet light so that a photograph could be taken. A strong band of genomic DNA on the gel was an indication that the majority of the DNA was not degraded; a smear on the gel was an indication that the DNA was largely degraded and possibly not useful for further testing. Those that were largely degraded were not used for further testing. Polymerase chain reaction (PCR) was used to assess the quality of the DNA as a template for amplification. The post-PCR DNA was analyzed by agarose gel electrophoresis on a 0.8% agarose gel containing 1 μg/ml of ethidium bromide. A strong band of amplified DNA on the gel was an indication that the DNA was of a high enough quality to be used in amplification reactions; the lack of such a band was an indication that the DNA was not useful for further testing. It was found that the presence of a large band of genomic DNA on the gel containing the purified pre-PCR DNA was a good predictor of success in the subsequent amplification reaction. Hence, for some samples, the subsequent PCR assessment was omitted.

### Example 4

A portion of each DNA sample was stored at −80° C. as a back-up sample, while the remainder of each DNA sample was subjected to a “normalization” procedure to equilibrate the DNA concentrations of each DNA sample. After normalization, the samples were also tested for population stratification so that a correction could be applied to get an equal population structure value for each pooled sample. Stratification and correction assays are further described in U.S. patent application Ser. No. 10/427,696, filed Apr. 30, 2003, and PCT patent application Ser. No. US04/013577, filed Apr. 30, 2004, both of which are entitled “Method for Identifying Matched Groups”. Equal volumes from each case sample were pooled together to form a “case pool;” and equal volumes of each control sample were pooled to form a “control pool.” Remaining portions of case or control samples were stored at −80° C.

### Example 5

The case pool and control pool were each separated into three equal pools for a total of six pools, (e.g., three identical case pools and three identical control pools). Each pool was separately subjected to long-range PCR using primers designed to amplify genomic DNA containing single nucleotide polymorphisms (SNPs). In total, over 1.7 million SNPs were amplified Methods for long-range PCR are disclosed, for example, in U.S. patent application Ser. No. 10/042,406, filed Jan. 9, 2002, entitled “Algorithms for Selection of Primer Pairs”; U.S. patent application Ser. No. 10/236,480, filed Sep. 9, 2002, entitled “Methods for Amplification of Nucleic Acids”; and U.S. Pat. No. 6,740,510, issued May 25, 2004, entitled “Methods for Amplification of Nucleic Acids”. Briefly, the PCRs were performed in 384-well plates containing primer pairs to which PCR reaction cocktail, DNA template (one of the pools discussed supra), a Taq antibody (and its buffer), and a long-range DNA Polymerase were added. The final DNA concentration in the PCR was 100 ng/μl. The PCR plates were sealed prior to PCR. Long-range PCR was performed for approximately 13.5 hours. The thermocycler block was allowed to reach 90° C. before the PCR plates were placed in the thermocycler. The rmocycler program used for the PCR is identified in Table 1:

TABLE 1

| Step | Action |
|---|---|
| 1 | Incubate at 95° C. for 3 min |
| 2 | Incubate at 94° C. for 2 seconds |
| 3 | Incubate at 64° C. for 15 minutes |
| 4 | goto [step] “2” (for 10 subsequent cycles) |
| 5 | Incubate at 94° C. for 2 seconds |
| 6 | Incubate at 64° C. for 15 minutes* |
| 7 | goto [step] “5” (for 28 subsequent cycles) |
| 8 | Incubate at 62° C. for 60 minutes |
| 9 | Hold at 4° C. |

*increased by 20 seconds for each subsequent cycle

### Example 6

The post-PCR pools were purified using a commercially available centrifugal filter device. Using a spectrophotometer, the concentration of each post-PCR pool was measured twice, once for a 1:200 fold dilution and once for a 1:300 fold dilution. These two measurements were then averaged to get a final concentration. Then, each pool was diluted to achieve a final DNA concentration of approximately 1.5 μg/μl. If the concentration of the pool was between 1.3 μg/μl and 1.7 μg/μl, the pool was considered to be close enough to 1.5 μg/μl and the concentration was not adjusted. If the pool had a concentration above 1.7 μg/μl, then enough molecular grade water was added to lower the concentration to 1.5 μg/μl. If the pool had a concentration of less than 1.3 μg/μl then it was concentrated to raise the concentration to 1.5 μg/μl using a commercially available concentrating centrifugal filter device. Finally, the concentration of each ˜1.5 μg/μl pool was rechecked using a spectrophotometer.

To check the quality of the post-PCR pools, aliquots of each were subjected to agarose gel electrophoresis in a 0.8% agarose gel containing 1 μg/ml ethidium bromide submerged in 1× TBE buffer. Aliquots containing approximately 3 μg of amplified DNA were added to loading buffer prior to being transferred to wells in the gel. Controls such as a commercially available DNA ladder and a known quantity of genomic DNA were also included on the gel. The gel was run at 250-275 volts for approximately 30 minutes and then photographed while illuminated by UV light. A post-PCR pool was deemed to be of good quality if the brightness of its band on the gel approximated that of the genomic DNA control.

### Example 7

Post PCR-pools were subjected to fragmentation by DNase I digestion. Each fragmentation reaction was performed in a 2 ml Eppendorf tube as follows. First, a buffered solution containing 0.0029 U/μl DNase I was added to 9.6 μg of post-PCR DNA in a total volume of 37 μl and placed at 37° C. for approximately eight minutes. Then the reaction was transferred to a 95° C. heat block for 10 minutes to denature the DNase I. After DNase I denaturation, the Eppendorf tube was placed on ice for at least five minutes and any condensation on the walls of the tube was spun down using a picofuge.

The success of each fragmentation reaction was examined by gel electrophoresis. Two microliters of each fragmentation reaction was added to 8 μl of gel-loading dye, and 5 μl of the mixture was loaded onto an Invitrogen-Novex Precast gel (4-20% TBE gel) in 1× TBE buffer. A DNA ladder was also loaded onto the gel. Electrophoresis was performed at approximately 80 volts until the samples had migrated out of the wells (approximately five minutes), and the voltage was then increased to 132-146 volts for approximately 40 minutes. The gel was stained with 1× TBE containing 0.01% ethidium bromide for one minute at room temperature. Finally, the gel was photographed while being illuminated with UV light. For a fragmentation reaction to be deemed of good quality, the reaction appeared as a “smear” of fragments with the majority of the fragments between 40 and 100 base pairs in length. If the fragmentation reaction appeared to be of good quality, the next step was a labeling reaction as described below.

### Example 8

First, 1.5 μl of biotin mix stock (1 mM stock consisting of 0.5 mM of each of biotin 16-dUTP and biotin 16-ddUTP) was added to each tube containing a completed fragmentation reaction of good quality. Next, 1 μl (25 units) of native TdT (terminal transferase) (Boehringer Mannheim) or 1 μl (200 units) of recombinant TdT (Roche) was added to each tube. The fluid in the tubes was mixed and spun down in the picofuge prior to placement in a preheated thermocycler. The labeling reactions were incubated at 37° C. for 90 minutes, then at 95° C. for 10 minutes, and finally held at 4° C.

### Example 9

Each fragmented, labeled, post-PCR pool was applied to a microarray containing oligonucleotides complementary to the genomic DNA that was amplified. Both strands of the amplified PCR product were probed for approximately 1.7 million polymorphisms across the genome using microarray oligonucleotide probes. Since there are generally two alleles for a given polymorphic locus, the microarray contained both alleles of the complementary oligonucleotides at each polymorphic position so that the amplified DNA could be screened for both alleles of a given polymorphism simultaneously. Minor allele frequencies that varied significantly between the case group and control group were characterized as being associated with related disease. Results were verified by genotyping individual samples for polymorphisms that were potentially associated with the case or control group based on the pooled analysis.

Prior to application to an microarray, 37.5 μl of a labeled, pooled sample were combined with 187.5 μl of a hybridization solution comprising 130 μl 5M TMACl (tetramethylammonium chloride), 2.2 μl 1M Tris (pH 8), 2.2 μl 1% Triton X-100, 2.2 μl 5 nM control oligo b-948, 2.2 μl 10 mg/ml herring sperm DNA, and 48.7 μl H2O. This mixture (225 μl total volume) was heated for 10 minutes at 95° C., spun down in a picofuge, and placed in a thermocycler where it was incubated at 95° C. for 10 minutes, then held at 50° C. Then, 200 μl of the pooled sample was transferred to a microarray that had been warmed at 50° C. The microarray containing the pooled sample is placed in a 50° C. hybridization oven where it is rotated at 25 r.p.m. overnight (14 to 19 hours) such that the pooled sample is allowed to flow freely over the microarray during the incubation.

### Example 10

After incubation (i.e., hybridization), the microarray was removed from the hybridization oven and the sample was removed and stored at −20° C. Then, the microarray was washed 1-2× with 200 μl of 1× MES/0.01% Triton X-100. The microarray was inverted several times to ensure that the wash solution moved freely over the surface of the microarray prior to removing the wash solution by vacuum suction.

Next, 200 μl of the “First Stain Solution” (174 μl of 1× MES/0.01% Triton X-100, 25 μl of 20 mg/ml of acetylated BSA, and 1 μl of 1 mg/ml streptavidin) was added to each microarray. The microarray was inverted several times to ensure that the First Stain Solution moved freely over the surface of the microarray. Then, the microarray was rotated at 25 r.p.m. for 15 minutes at room temperature. Next, the microarray was washed with 1× MES/0.01% Triton X-100 wash solution in a Perlegen RevD Fluidics Station. When the wash was finished the microarray was removed from the fluidics station and the 1× MES/0.01% Triton X-100 wash solution was removed by vacuum suction.

Next, 200 μl of the “Second Stain Solution” (175 μl of 1× MES/0.01% Triton X-100, 25 μl of 20 mg/ml acetylated BSA, and 0.5 μl of 0.5 mg/ml biotinylated anti-streptavidin) was added to each microarray. The microarray was inverted several times to ensure that the Second Stain Solution moved freely over the surface of the microarray. Then, the microarray was rotated at 25 r.p.m. for 15 minutes at room temperature. Next, the microarray was washed with 1× MES/0.01% Triton X-100 wash solution in a RevD Fluidics Station. When the wash was finished the microarray was removed from the fluidics station and the 1× MES/0.01% Triton X-100 wash solution was removed by vacuum suction.

Then, 200 μl of the “Third Stain Solution” (174 μl of 1× MES/0.01% Triton X-100, 25 μl of 20 mg/ml acetylated BSA, and 1 μl of 0.2 mg/ml streptavidin Cy-chrome) was added to each microarray. The microarray was inverted several times to ensure that the Third Stain Solution moved freely over the surface of the microarray. Then, the microarray was rotated at 25 r.p.m. for 15 minutes at room temperature. Next, the microarray was washed with 1× MES/0.01% Triton X-100 wash solution in a RevD Fluidics Station. When the wash was finished the microarray was removed from the fluidics station and the 1× MES/0.01% Triton X-100 wash solution was removed by vacuum suction.

Then, a wash solution of 6× SSPE/0.01% Triton X-100 was added to the microarray. The microarray was inverted several times to ensure that the 6× SSPE/0.01% Triton X-100 moved freely over the surface of the microarray before it was removed by vacuum suction. Next, a wash solution of 0.2× SSPE/0.005% Triton X-100 that had been prewarmed to 37° C. was added to the microarray, which was then incubated at 37° C. for 30 minutes. The 0.2× SSPE/0.005% Triton X-100 was removed by vacuum suction and a solution of 1× MES/0.01% Triton X-100 was added to the microarray. The microarray was then inverted several times before the 1× MES/0.01% Triton X-100 was removed by vacuum suction. Finally, fresh 1× MES/0.01% Triton X-100 was added to the microarray, which was wrapped in foil prior to storage at 4° C. or scanning of the microarray.

### Example 11

On the same days the microarrays were stained and washed, they were scanned using an arc scanner. After scanning, the microarrays were removed from the scanner, wrapped in foil and stored at 4° C. The scan files generated by the scanner were then analyzed by software programs designed to interpret intensity data from microarrays. This software allowed discrimination of hybridization patterns that distinguished the case pools from the control pools. The data were analyzed according to the methods disclosed in the following U.S. patent applications, all of which are assigned to the assignee of the present applications: U.S. provisional patent application No. 60/460,329, filed on Apr. 3, 2003, entitled “Apparatus and Methods for Analyzing and Characterizing Nucleic Acid Sequences”; and U.S. patent application Ser. No. 10/768,788, filed Jan. 30, 2004, entitled “Apparatus and Methods for Analyzing and Characterizing Nucleic Acid Sequences”. Nucleic acids that were identified as strongly associated with the case or control group based on the pooled genotyping analysis were reanalyzed by genotyping individual samples for those potentially associated nucleic acids, as described below. As such, individual genotyping was performed on approximately 30,000 (˜2%) of the original 1.7 million SNPs.

### Example 12

A sample from each individual was subjected to a plurality of multiplex (˜78-plex), short-range PCRs using primers designed to amplify genomic DNA containing approximately 30,000 potentially associated nucleic acids (e.g., SNPs) that were identified in the pooled genotyping methodology as described supra. The PCRs were performed in 384-well plates containing DNA template (10 ng) and PCR cocktail (1.47 μl 10× AK2 buffer (0.5M Trizma, 0.14M ammonium sulfate, and 27 mM MgCl2), 0.03M tricine, 0.67 μl MasterAmp 10× PCR Enhancer (Epicentre, Madison, Wis.), 3.9% DMSO, 0.05M KCl, dNTPs (0.54 mM each), PCR primers (0.42 pmol/μl/primer), and ˜2× Titanium Taq polymerase (BD Biosciences, Palo Alto, Calif.)). The PCR plates were led prior to PCR. Short-range PCR was performed for approximately three hours. The thermocycler block was allowed to reach 90° C. before the PCR plates were placed in the thermocycler. The thermocycler program used for short-range PCR is identified in Table 2:

TABLE 2

| Step | Action |
|---|---|
| 1 | Incubate at 96° C. for 5 min |
| 2 | Incubate at 96° C. for 30 seconds |
| 3 | Incubate at 58° C. (−0.5° C./cycle) for 30 seconds |
| 4 | Incubate at 65° C. for 60 seconds |
| 5 | goto [step] “2” (for 9 subsequent cycles) |
| 6 | Incubate at 96° C. for 10 seconds |
| 7 | Incubate at 53° C. for 30 seconds |
| 8 | Incubate at 65° C. for 60 seconds |
| 9 | goto [step] “6” (for 43 subsequent cycles) |
| 10 | Incubate at 65° C. for 7 minutes |
| 11 | Hold at 4° C. |

Once the PCR is complete, the plates were removed from the thermocycler and were pooled as described infra. (At this point, the plates could also have been stored at −20° C. for an extended period, if so desired.)

PCR plates containing amplified sample were spun at 1000 r.p.m. for 15 seconds in a table-top Sorvall centrifuge. Amplified samples from a single individual corresponding to a single chip (microarray) design were pooled together. The pooled samples were then arrayed into 96-well plates and quantified using PicoGreen reagent (Molecular Probes, Inc., Eugene, Oreg.) and a SpectraFluor Tecan Plate Reader (Tecan Group Ltd., Maennedorf, Switzerland). Amplified samples that contained less than 100 ng/μl were deemed to have failed PCR and were not analyzed further.

### Example 13

Post-PCR pools were subjected to treatment with shrimp alkaline phosphatase (SAP). Each treatment was performed in a well of a 96-well plate and contained 8 μg amplified sample, 5 U SAP (Promega, Madison, Wis.), and ˜1× One Phor All buffer Plus (Amersham Biosciences, Buckinghamshire, England) in a total volume of 100 μl. The reaction mixture was incubated at 37° C. for 30 minutes, 80° C. for 20 minutes, and then cooled to 4° C. The SAP-treated samples were then labeled with biotin. (At this point, the SAP-treated sample could be stored overnight at −20° C. prior to biotin-labeling.)

### Example 14

The SAP-treated pools were labeled with biotin. Each labeling reaction was performed in one well of a 96-well plate and contained the 100 μl volume of the SAP-treated pool plus 3 μl of 0.5 mM biotin d/dd-UTP and 800 U of recombinant TdT. The plate was sealed, vortexed briefly, and centrifuged at 1000 r.p.m. for 15 seconds in a table-top Sorvall centrifuge. The plate was placed in a thermocycler and incubated at 37° C. for 90 minutes, 99° C. for 10 minutes, and then cooled to 4° C. The biotin-labeled pools were hybridized to microarrays on the same day as they were labeled.

### Example 15

Hybridization buffer (1.5M TMACL (tetramethylammonium chloride), 5 mM Tris (pH 7.8 or 8.0), 0.005% Triton X-100, 26 pM b-948 control oligo (Genset, La Jolla, Calif.), and 0.05 mg/ml HS (herring sperm) DNA) was prewarmed at 60° C. for a minimum of 30 minutes. Microarrays (e.g., chips) were prewarmed at 50° C. in a hybridization oven for approximately 30 minutes. 195 μl of hybridization buffer was added to each well of a 96-well plate that was prewarmed at 60° C. for a minimum of 30 minutes, and the plate (“hybridization plate”) was sealed and returned to the heat block. The 96-well plate containing the labeled sample was centrifuged at 1000 r.p.m. for 15 seconds in a table-top Sorvall centrifuge prior to heating the plate at 99° C. for 10 minutes and subsequently cooling the plate to 60° C. (for no more than 5 minutes) in order to denature the labeled sample. Once the denaturation is complete, the denatured samples (105 μl) were transferred to wells on the hybridization plate containing the 195 μl aliquots of hybridization buffer, and were mixed by pipetting the solution up and down twice. The hybridization plates were resealed and returned to the 60° C. heat block.

The mixture containing the denatured samples and hybridization buffer was transferred to a prewarmed microarray. The array was sealed, returned to the 50° C. hybridization oven, and rotated at 20 r.p.m. overnight (14-19 hours). After the overnight incubation, the array was stained, washed and scanned as described for the pooled genotyping methodology, supra.

After scanning, the microarrays were removed from the scanner, wrapped in foil and stored at 4° C. The scan files generated by the scanner were then analyzed by software programs designed to interpret intensity data from microarrays. This software assigned genotypes at each SNP position for each individual in the case and control groups. The data were analyzed according to the methods disclosed in the following U.S. patent applications, all of which are assigned to the assignee of the present applications: U.S. patent application Ser. No. 10/351,973, filed Jan. 27, 2003, entitled “Apparatus and Methods for Determining Individual Genotypes”; and U.S. patent application Ser. No. 10/786,475, filed Feb. 24, 2004, entitled “Improvements to Analysis Methods for Individual Genotyping”. The nucleic acids listed in Appendix 1, were identified as strongly associated with the case or control group. The following is a description of the column headings for Appendix 1.

TABLE 3A
COLUMN IDENTIFIERS FOR APPENDIX 1 (TABLE 3B)

| Column Name | Description |
|---|---|
| SNP_ID | SNP identifier. Perlegen SNP Identifiers may be used for |
| | accessing additional information about the SNP using the |
| | Genotype Browser on the Perlegen Sciences, Inc. website |
| | (genome(dot)perlegen(dot)com/browser/index.html). |
| Chromosome | Chromosome number of the NCBI Build 34 contig on which the |
| | best alignment was found. 23 is used for the X chromosome, 24 |
| | for the Y chromosome. |
| Contig | The accession number from NCBI Build 34 of the contig to |
| | which the SNP aligns. |
| Location | Nucleotide position in NCBI Build 34 contig of the SNP base in |
| | the alignment. |
| sequence | The 29mer assayed for this SNP, with the ref allele and alt allele |
| | in square brackets representing the SNP at the middle base. |
| ref allele | Reference allele. |
| alt allele | Alternate allele. |
| allele highest in cases | The allele (ref or alt) that is found at a higher frequency in cases |
| | relative to controls. |
| Genename | The Gene name from the NCBI Entrez Gene database |
| | (http://www.ncbi.nlm.nih.gov/entrez/query.fcgi?db=gene) |
| geneID | The GeneID from the NCBI Entrez Gene database. |
| geneDescription | The Gene description from the NCBI Entrez Gene database. |

### Example 16

This Example relates to a Genome-wide association study for Olanzaine Treatment-Emergent Weight Gain. Treatment-emergent weight gain observed with atypical antipsychotic therapy continues to be a clinical concern with genetic factors likely playing a role. The genetic contribution to weight gain has been investigated using a candidate gene approach (reviewed by Muller et al. 2004). Although significant associations with candidate genes such as the Serotonin 5-HT_{2c} Receptor Gene (Reynolds et al. 2002) and CYP2D6 (Ellingrod et al. 2002) have been reported, negative results have also been described (Muller et al. 2004, Hong et al. 2001). The lack of consistent findings has led to uncertainty as to the significance of reported associations. Therefore, we undertook a large scale effort to investigate many genes across the genome in a large cohort of patients for treatment emergent weight gain.

Overview

Treatment-emergent weight gain observed with atypical antipsychotic therapy continues to be a clinical concern, with genetic factors likely playing a role. The genetic contribution to weight gain has been investigated using a candidate gene approach (reviewed by Muller et al. 2004). Although significant associations with candidate genes such as the Serotonin 5-HT2c Receptor Gene (Reynolds et al. 2002) and CYP2D6 (Ellingrod et al. 2002) have been reported, negative results have also been described (Muller et al. 2004, Hong et al. 2001). The lack of consistent findings has led to uncertainty as to the significance of reported associations. Therefore, we undertook a large-scale effort to investigate many genes across the genome in a large cohort of patients for treatment-emergent weight gain.

Using a cohort of adult patients diagnosed with schizophrenia, schizoaffective, or schizophreniform disorder who had taken oral olanzapine for a minimum of six months, case-control populations were chosen from the tails of the weight-gain distribution. The cases (n=258) represented the 20% extreme weight gainers, and the controls (n=255) consisted of the 20% of the individuals who gained the least weight (nongainers), both measured by change in body mass index. Mean (±SD) body mass index for the weight gainers was 33.9±6.1 kg/m² and for the nongainers, 27.1±6.3 kg/m². A regression model with age, gender, and ethnicity as covariates was used to define the weight-gain distribution balancing the weight gainers and nongainers for these factors.

Phase I of the analyses involved pooling of the DNA for each group, weight gainers and nongainers. Each pool of DNA was genotyped for ˜1.7 million single nucleotide polymorphisms (SNPs) using the Perlegen Sciences platform. The allele frequency difference between the weight gainers' and nongainers' pools was calculated from three replicate determinations on each pool, for each of the SNPs genotyped. A total of 30,000 SNPs were then carried forward to phase II, in which all 513 individuals were individually genotyped. The 30,000 SNPs genotyped on each individual were chosen based on three criteria: 1) SNPs with the largest estimated allele frequency differences between the two pools (±0.084) (n=23,281 SNPs); 2) SNPs with estimated allele frequency differences of ±0.065 between pools but where the pooled data from multiple SNPs matched the expected correlational structure based on haplotypes as defined in Perlegen's haplotype map (n=5,000 SNPs); and 3) 1,719 SNPs from 47 candidate genes.

Association analyses between the 30,000 SNPs and weight-gain phenotype were completed using Fisher's exact test. Three hundred eleven SNPs were identified as significantly different (p<0.001, uncorrected for multiple testing) between weight gainers and nongainers. Bioinformatics tools, additional scoring algorithms, and statistical analyses were used to narrow the list to the most interesting SNPs and gene regions.

Methods

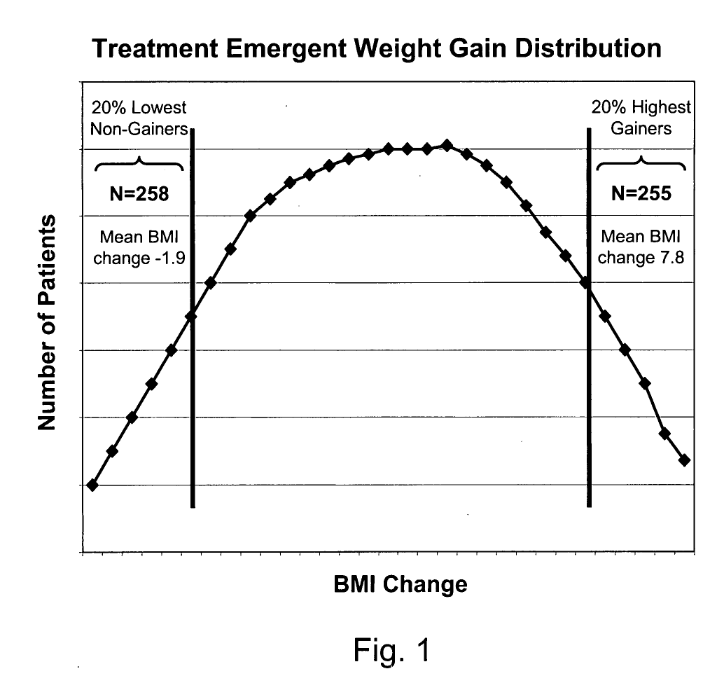

Using a cohort of adult patients diagnosed with schizophrenia, schizoaffective or schizophreniform disorder who took oral olanzapine for a minimum of six months, case-control populations were chosen from the tails of the weight-gain distribution. The cases (n=258) represented the 20% extreme weight gainers, and the controls (n=255) consisted of the 20% of the individuals who gained the least weight (nongainers), both measured by change in body mass index. Mean (±SD) body mass index for the weight gainers was 33.9±6.1 kg/m² and for the nongainers, 27.1±6.3 kg/m². A regression model tested for first and second order interactions with age, gender, and ethnicity as covariates was used to define the weight-gain distribution balancing the weight gainers and non-gainers for these factors. FIG. 1 provides a graph of treatment emergent weight gain distribution, in which the BMI (body mass index) change is charted against the total patient population, including the 20% lowest gainers (n=258) and the 20% highest gainers (N=255).

One major concern with pooling DNA across populations concerns the ethnic makeup of the two pools. If the same ethnic contribution is not present into each pool, genetic variants with different frequencies simply due to evolutionary factors and, therefore, with different representation in different ethnic groups, may be found significant, further inflating false positive findings. For that reason, phase I included testing for population stratification. A random set of 289 SNPs, equally distributed across the genome and displaying adequate heterogeneity between populations, were genotyped in each individual patient.

To detect association, 280 SNPs (Pritchard, et al., (2004) Genetics 155: 945-959; Hinds, et al. (2004)) were examined by the Pearson Chi-squared test of allele frequency differences between gainers and non-gainers (N=˜280):

TABLE 4

| | p < 0.0001 | p < 0.001 | P < 0.01 | P < 0.1 |
|---|---|---|---|---|
| # Expected | 0 | 0 | 3 | 28 |
| # Observed | 0 | 2 | 5 | 26 |

For automatic detection of genotype clusters, samples were assigned randomly to clusters and then reassigned by genotype until stable (see, e.g., Prichard et al. (1999) AJHG 65:220-228):

TABLE 5

| Reported Ethnicity | 1 | 2 | 3 |
|---|---|---|---|
| African American (%) | 3.3 | 8.4 | 88.3 |
| Cucasian (%) | 6.6 | 89.7 | 3.7 |
| Hispanic (%) | 70.5 | 26.0 | 3.5 |

TABLE 6

| | Study Group | | |
|---|---|---|---|
| | 1 | 2 | 3 |
| Control (%) | 14.0 | 61.9 | 24.1 |
| Case (%) | 13.8 | 64.4 | 23.8 |
| p-value (%) | 0.93 | 0.48 | 0.48 |

Stratification and cluster analysis of these SNPs in the 513 individuals selected for the two pools demonstrated that subpopulation structure (ethnic makeup) was not significantly different.

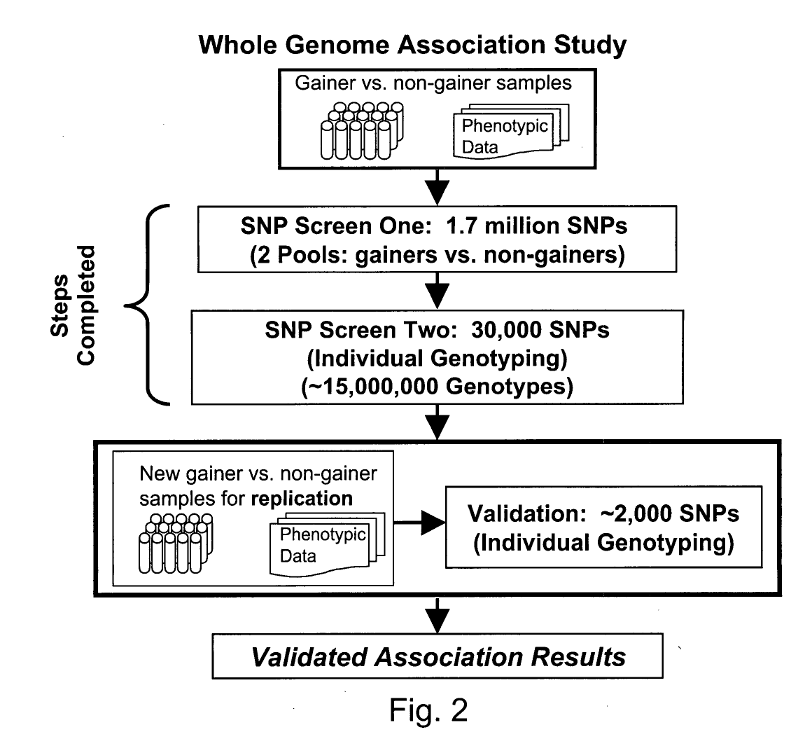

FIG. 2 shows a schematic overview of the whole genome association study used in this Example. Phase I of the analyses involved pooling of the DNA for each group, weight gainers and non-gainers. Each pool of DNA was genotyped for ˜1.7 million single nucleotide polymorphisms (SNPs) using the Perlegen Sciences platform. In order to reduce the technical variability and the number of false positives, the allele frequency difference between the weight gainers' and nongainers' pools was calculated from three replicate measures on each pool, for each of the SNPs genotyped.

TABLE 7
SNP Quality for Whole Genome Scan Phase

| | | |
|---|---|---|
| All SNPs | 1,717,004 | |
| Unique Build 33 positions | 1,619,649 | 94% |
| Has enough measurements | 1,517,840 | 88% |
| Has within pool standard | 1,441,653 | 84% |
| error <.04 | | |
| No competing hits (no close | 1,404,184 | 82% |
| match leading to cross | | |
| hybridization) | | |

Note:
“Unique Build 33 positions” refer to the number of SNPs within the total assayed set that mapped uniquely to the human genome (NCBI Build 33 refers to the particular version of the human genome to which the SNPs were mapped).

Note: “Unique Build 33 positions” refer to the number of SNPs within the total assayed set that mapped uniquely to the human genome (NCBI Build 33 refers to the particular version of the human genome to which the SNPs were mapped).

TABLE 8
OBSERVED SNPS FOR DEFINED FREQUENCY DIFFERENCES
Total number of SNPs 1,404,184
SNPs with frequency differences between gainers & non-gainers:

| approx freq diff | expected false+ | observed # of SNPs |
|---|---|---|
| >0.06 | 91,170 | 98,780 |
| >0.07 | 43,605 | 58,442 |
| >0.08 | 19,143 | 34,210 |
| >0.09 | 7,703 | 19,984 |

See also, Tables 11 and 12.

TABLE 9
SNPS CARRIED FORWARD TO INDIVIDUAL GENOTYPING

| Category | Cutpoint | # SNPs | % SNPs |
|---|---|---|---|
| LLY Candidate Genes | None | 1,719 | 5.70% |
| Haplotype Conforming | 0.065 | 5,000 | 16.70% |
| Non-conforming | 0.084 | 23,281 | 77.60% |
| Total | | 30,000 | 100.00% |

Note:
Regarding category information: In selecting a subset of SNPs from the pooled genotyping stage for individual genotyping, two board sets of SNPs were considered:
(a). SNPs within or in the vicinity of genes for which there was prior evidence of association with the phenotype of interest: all of these SNPs were selected, whether they showed evidence for association within the pooled genotyping data or not. SNPs selected in this manner fall in to category “LLY Candidate Genes” in the application, also noted as “SNPs in Candidate Genes”.
(b). SNPs that showed evidence for association in the pooled genotyping data: in selecting these, use was made of a human haplotype map derived earlier and independently at Perlegen Sciences (Patil et al, Science, 2001) to improve the quality of the SNP selection.

Note: Regarding category information: In selecting a subset of SNPs from the pooled genotyping stage for individual genotyping, two broad sets of SNPs were considered:

(a). SNPs within or in the vicinity of genes for which there was prior evidence of association with the phenotype of interest: all of these SNPs were selected, whether they showed evidence for association within the pooled genotyping data or not. SNPs selected in this manner fall into the category “LLY Candidate Genes” in the application, also noted as “SNPs in Candidate Genes”.

(b). SNPs that showed evidence for association in the pooled genotyping data: in selecting these, use was made of a human haplotype map derived earlier and independently at Perlegen Sciences (Patil et al, Science, 2001) to improve the quality of the SNP selection.

In regions where the haplotype map accurately represents the populations sampled in the study, it prescribes a linear relationship between the allele frequency differences for the SNPs within a haplotype block and the allele frequency differences of its common patterns. This relationship is tested for all haplotype blocks, using the pooled Dp-hat (estimated or approximate allele frequency difference) as a proxy for the true allele frequency differences. When the Dp-hat values for SNPs in a block are determined to conform to the haplotype map (p value <0.05 for a linear regression), the estimated differences in frequency between pools for the common haplotype patterns are used to generate “fitted” estimates of Dp-hat for the individual SNPs. These “fitted Dp-hat” values effectively average over redundant SNPs within each block and therefore improve allele frequency difference estimates. The redundancy also allows for greater effective coverage with a smaller selection, or the ability to examine smaller allele frequency differences with the same number of SNPs. The fitted Dp-hat is better correlated with the true allele frequencies as determined from individual genotyping than is Dp-hat itself.

SNPs that are members of haplotype blocks that conform to the haplotype map in the manner described above fall into the category “Haplotype conforming” in Table 9. SNPs that fall within haplotype blocks that do not conform to the Perlegen haplotype map, or that were not part of the Perlegen haplotype map at all, fall into the category “non-conforming”. The column currently labeled “cutoff” is the threshold in Dp-hat for selection, or the threshold in the estimated allele frequency difference from pooled genotyping. For the reasons described in the previous paragraph, a lower threshold for SNPs was used in conforming haplotype blocks than for other SNPs.

Phase II involved selecting 30,000 SNPs for individual genotyping and further analyses. Association analyses between the SNPs genotyped in phase II and weight-gain phenotype were completed using Fisher's exact test. Bioinformatics tools, additional scoring algorithms, and statistical analyses were used to narrow the list to the most interesting SNPs and gene regions. Results are shown below:

TABLE 10
PHASE 2 INDIVIDUAL GENOTYPING RESULTS

| | |
|---|---|
| SNPs per Sample | 30,000 |
| Sapmles | 513 |
| SNPs with call in >50% samples | 96% |
| Call rate across good SNPs | 97% |
| Replicate concordance (n = 1.2M) | 99.8% |
| Concordance w/stratification data (n = 50k) | 99.1% |
| Total assigned genotypes | 14,225,757 |

TABLE 11
TOP SNPS IDENTIFIED USING FISHER'S EXACT

| Exact P value | # of SNPs | Range of RR |
|---|---|---|
| <0.001 | 290 | 8.70-1.23 |
| 0.001-0.005 | 825 | 4.31-1.19 |
| 0.005-0.01 | 749 | 378-1.18 |

“RR” is the relative risk: the ratio of the risk for displaying the phenotype among individuals carrying one copy of the predisposing allele to the risk among individuals who do not carry the predisposing allele.

TABLE 12
Second Phase Significant Genes

| Gene | Locus | # of SNPs | # of SNPs | Total SNPs |
|---|---|---|---|---|
| Symbol | (gene ID)* | p < .001 | .001 < p < .01 | Tested |
| PKHD1 | 5314 | 9 | 5 | 45 |
| NRXN3 | 9369 | 6 | 7 | 21 |
| PAM | 5066 | 4 | 6 | 10 |
| EPHA7 | 2045 | 4 | 4 | 11 |
| ROS1 | 6098 | 4 | — | 7 |
| None | 341547 | 3 | 2 | 6 |
| FKSG87 | 83953 | 3 | 1 | 5 |
| C3orf6 | 152137 | 3 | 6 | 17 |
| None | 378045 | 3 | 1 | 7 |
| | (375553) | | | |
| TOX | 9760 | 3 | 5 | 23 |
| DLG2 | 1740 | 3 | 3 | 21 |
| MDS1 | 4197 | 3 | — | 12 |
| PAPPA | 5069 | 3 | — | 8 |
| FABP2 | 2169 | 3 | — | 5 |
| EFA6R | 23362 | 3 | — | 11 |
| FLJ20125 | 54826 | 3 | 4 | 6 |
| C1orf10 | 49860 | 2 | 1 | 3 |
| CHL1 | 10752 | 2 | 4 | 13 |
| BICD1 | 636 | 2 | 3 | 11 |
| KREMEN1 | 83999 | 2 | 1 | 6 |
| ADARB2 | 105 | 2 | 1 | 7 |
| KCNMA1 | 3778 | 2 | 1 | 11 |
| A2BP1 | 54715 | 2 | 4 | 59 |
| None | 374942 | 2 | — | 4 |
| MGC4309 | 79098 | 2 | — | 2 |
| PIGR | 5284 | 2 | — | 4 |
| PCSK7 | 9159 | 2 | — | 4 |
| HSF2 | 3298 | 2 | — | 3 |

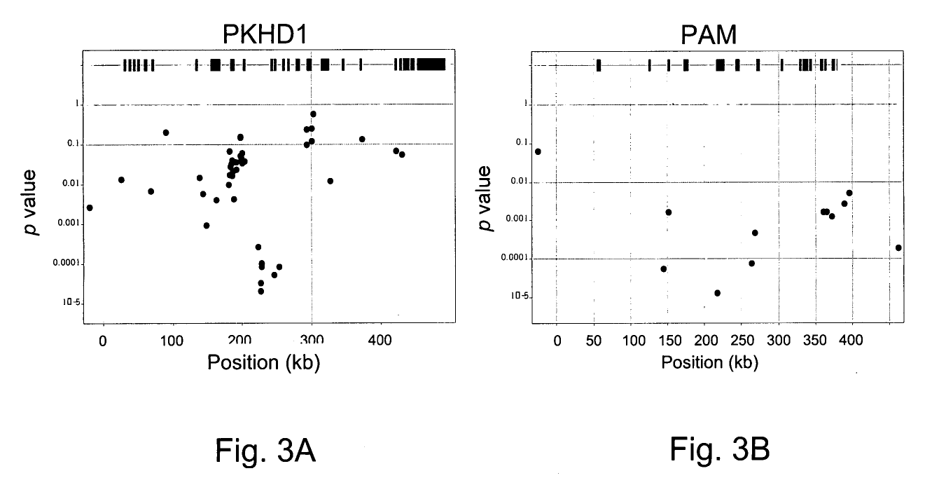

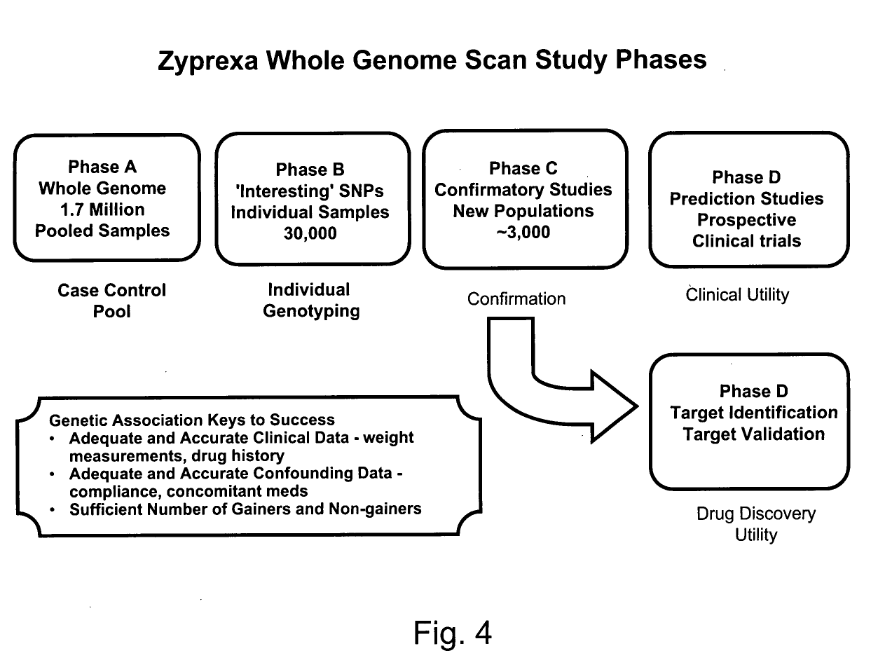

FIG. 3 shows representative scatter plots for PKHD1 and PAM, two of the genes identified as having SNPs that correlate with weight gain in the second phase study, with p value on the y-axis and the position that a given SNP maps to within the gene on the x-axis. FIG. 4 provides a schematic outline of an overall Zyprexa (olanzapine) whole genome scan study.

### REFERENCES

Ellingrod V L, Miller D, Schultz S K, Wehring H, Arndt S. CYP2D6 polymorphisms and atypical antipsychotic weight gain. Psychiatr Genet 2002;12:55-58.

Hinds D A, Stokowski R P, Patil N, Konvicka K, Kershenobich D, Cox D R, Ballinger D G. Matching Strategies for Genetic Association Studies in Structured Populations. Am. J. Hum. Genet. 74:317-325, 2004.

Hong C J, Lin C H, Yu Y W, Yang K H, Tsai S J. Genetic variants of the serotonin system and weight change during clozapine treatment. Pharmacogenetics 2001;11:265-268.

Muller D J, Muglia P, Fortune T, Kennedy J L. Pharmacogenetics of antipsychotic-induced weight gain. Pharmacol Res 2004;49:309-329.

Reynolds G P, Zhang Z, Zhang X B. Association of antipsychotic drug-induced weight gain with 5-HT2c receptor gene polymorphism. Lancet 2002;359:2086-2087.

### Example 17

### The Role of Genes Including PKHD1 in Atypical Anti-Psychotic Treatment Emergent Weight Gain

Atypical anti-psychotic treatment-emergent weight gain is of clinical concern and, to date, the mechanistic cause for this treatment effect is unknown. Novel genes associated with weight gain were identified through a whole-genome association study on patients exposed to olanzapine as discussed above. These findings were replicated in a cohort of parent-child trios where the probands were selected for an obese phenotype. The genes involved associated both with the weight gain and obese phenotype included PKHD1. Abnormal fat metabolism in the PKHD 1 knockout mouse further confirm this gene's role in adiposity, highlighting the previously underestimated importance of cilia function to fat deposition.

Treatment emergent weight gain observed with atypical antipsychotic therapy is a clinical concern, as many patients are currently being prescribed such medications. In a meta-analysis of antipsychotic agents used over a 10-week treatment period, mean weight gain during treatment with olanzapine was 4.15 kg, and during treatment with clozapine was 4.45 kg. In addition to the well described adverse medical sequelae of excessive weight gain (e.g., heart disease, diabetes), weight gain in schizophrenia has also been linked to poor quality of life and medication noncompliance. Although the mechanism underlying treatment emergent weight gain remains largely unknown, genetic influences have been proposed. The genetic contribution has been investigated using candidate gene approaches, with contradictory results, leading to uncertainty as to the significance of any gene's involvement (Muller et al. (2004) “Pharmacogenetics of Antipsychotic-induced Weight Gain” Pharmacological Research 49:309-329; Reynolds et al. (2002) “Association of antipsychotic drug-induced weight gain with 5-HT_{2c} Receptor Gene Polymorphism,” Lancet 359:2086-2087; Ellingrod et al. (2002) “Polymorphisms and Atypical Antipsychotic Weight Gain” Psychiatric Genetics 12:55-58; Hong et al. (2001) “Genetic Variants of the Serotonin System and Weight Change during Clozapine Treatment” Pharmacogenetics 11:265-268). Candidate gene studies have focused on those genes implicated in neuro-physiological functioning, regulation of weight homeostasis and/or food satiety, and the pharmacological action and disposition of atypical anti-psychotics. With the recent practicality of whole genome scanning technologies, and to shed light on the potential genetic mechanism(s) involved in this phenomenon beyond what can be hypothesized, a whole-genome SNP association study and replication in an independent cohort, followed by functional observation in a knockout mouse were completed.

Treatment emergent weight gain whole-genome association study. The first stage, a whole-genome association study of treatment emergent weight gain, was selected to allow investigation of mechanisms we cannot hypothesize and eliminate the typical bias towards known biology. This study involved two phases, quantitative pooled genotyping of greater than 1.4 million single nucleotide polymorphisms (SNPs), followed by individual genotyping of nearly 30,000 SNPs that displayed the highest significance out of the 1.4×10⁶ SNPs. The cohort was the 20% extreme weight gainers and non-gainers as measured by change in body mass index of a population of patients diagnosed with schizophrenia, schizoaffective or schizophreniform disorder that took oral olanzapine for a minimum of six months.

Prior to pooling, a predefined set of 289 SNPs was genotyped to test for population substructure bias (see also, Hinds et al. (2004) “Matching Strategies for Genetic Association Studies in Structured Populations” Amer Journal of Human Genetics, 74(2): 317-325). No indication of confounding due to population substructure bias was noted. Phase 1 of the association analysis with weight gain involved quantitative pooling of the DNA separately for the weight gainers and non-gainers and calculation of estimated allele frequency differences (Δₚₕₐₜ) between the pools for each of the 1.4×10⁶ SNPs as described (Hinds et al. (2004) “Application of Pooled Genotyping to Scan Candidate Regions for Association with HDL Cholesterol Levels” Hum Genomics 1(6):421-434). Phase 2 included genotyping all individuals for 28,281 SNPs: 23,281 SNPs with the largest Δₚₕₐₜ between the two pools (Δₚₕₐₜ≧0.084); and 5,000 non-redundant SNPs with Δₚₕₐₜ between pools (Δₚₕₐₜ≧0.065) but where the pooled data from multiple SNPs matched the expected correlational structure based on haplotypes as defined in Perlegen's haplotype map. SNPs were tested for association with weight gain after removing those that failed assay development, Hardy Weinberg equilibrium tests, or without sufficient observations. The association analyses identified 290 SNPs from 107 genes as significantly different between weight gainers and nongainers (Fisher's exact p-value<0.001) (note in references on method and data in supplemental materials). Several genes had multiple significant SNPs.

Similar to mRNA microarray, proteomic, and metabonomic studies, large scale genetic studies yield large data sets that can include false positive results. Without independent confirmation, true positive results are difficult to distinguish. However, several genes from these studies displayed a clustering of SNPs across several haplotype and linkage disequilibrium blocks, making it likely that these observations are significant. These SNPs fall within the gene boundaries of genes such as PAM, FABP2 and PAPPA where involvement in weight gain have been hypothesized as discussed herein. Genes with the strongest clustering results, such as PKHD1, ROS₁, TOX and NRXN3, were not previously implicated in a mechanism of weight control.

An independent additional collection of samples from patients with atypical anti-psychotic associated treatment emergent weight gain was not immediately available from academic or commercial sources to confirm these novel genetic associations. However, based on what has been discovered about the dual role for central and peripheral mechanisms of weight maintenance, it is expected that the genetic influences of treatment emergent weight gain and generalized obesity may overlap. Therefore, the initial replication was performed on a cohort selected for obesity.

Replication in an obese cohort. Parent-child trios (n=348) where the probands, the child in this case, was selected for an obese phenotype, BMI>35 , were genotyped to replicate the findings. A chip containing 3741 SNPs was designed using the following SNP selection strategy: all SNPs significant from phase 2 of the genome wide association study at p<0.002 (n=668); and additional SNPs chosen both for redundancy near the significant SNP and for non-redundant SNPs representative of the linkage disequilibrium bins surrounding the significant SNP (n=3073). 3286 of these 3741 SNPS (88%) were successfully assayed and used for association with obesity using the Transmission Disequilibrium Test (TDT). SNPs representing 13 of the 107 genes carried forward had at least one SNP replicate at p<0.01 in the obesity cohort.

Although we proposed a common mechanism between generalized obesity and treatment emergent weight gain, the number of genes that replicated between the treatment emergent weight gain cohort and the obesity cohort was unexpected. While at least one of the genes is in a metabolic pathway, FABP2, where linkage to maintenance of body mass seems reasonable, the rest of the genes that replicated had no previously known link to adiposity. The replication in an unrelated obesity cohort, however, strongly implies that genes such as PKHD1, EPHA7, INPP4B and LAMA4 are involved in weight control.

PKHD1 knockout mouse. In independent efforts, a mouse knockout model, where conserved exons 3 and 4 were removed, was developed to investigate the impact of PKHD1 on polycystic liver and kidney disease. Consistent with polycystic kidney disease in humans, PKHD1 exon 3 and 4 knockout animals have a varying degree of overt symptomatology. The animals show clear signs of kidney, pancreas, or liver disease, and manifest a smaller body mass due to the disease. In contrast, in those homozygote knockout animals who were not overtly sick, an abnormal visceral fat deposition was noted. This type of fat deposition has not been found in genetically unmanipulated animals of these strains, nor reported in the literature. Since the disease phenotype is variable in both humans and animals, possibly due to the size of the gene and the multiple, alternate exon transcripts seen in this gene, it was not unexpected that not all homozygous KO animals would manifest abnormal visceral fat deposits.

To investigate if the manifestation of abnormal fat metabolism required both copies of the gene, heterozygous animals were investigated for fat deposits. Unlike polycystic disease symptoms, heterozygotes displayed a generalized obese phenotype, weighing roughly twice what their homozygous wt littermates weighed. These observations confirm that an alteration in cilia function in a mouse knockout model leads to abnormal fat deposition, and provides a biological link to the observed SNP association information.

The finding that gene(s) involved with cilia structure and function are involved in treatment emergent weight gain and obesity was unexpected. However, the evidence for PKHD1's involvement in treatment emergent weight gain and generalized obesity is now substantial. The whole-genome association study revealed a cluster of SNPs within PKHD1 significantly associated with weight gain. Eight SNPs within a 50 kb region, spanning three independent haplotype blocks, were significant without a non-significant SNP interspersed. This alone made the likelihood of PKHD1 involvement in treatment emergent weight gain quite high. The independent replication in an obese population further strengthened this association, and suggested that PKHD1 also has involvement in fat metabolism, independent of atypical antipsychotic treatment. The direct link to biology provided by the PKHD1 KO mouse model further demonstrates the involvement of PKHD 1 in obesity and related metabolic defects.

As is true for most complex phenotypes, multiple genetic mechanisms underlie the basis for weight gain. The discovery of common mechanisms involving novel biology is compelling. This example demonstrates an ability to discover novel genetic mechanisms using a genome wide association study, replication and biological investigation. PKHD1, FABP2, EPHA7, INPP4B and LAMA4 were associated again in the obesity cohort. It is not clear yet how all of these genes are involved in metabolism and maintenance of body weight.

One additional association between the candidate genes and obesity may be shown by the congenital polycystic disease called Bardet-Beidl syndrome. In this rare form of polycystic kidney disease, obesity is prominent. However, it is unclear how the common form of autosomal dominant polycystic kidney disease that involves PKHD1 relates to Bardet-Beidl, where 6 different genes have been implicated.

Although we propose a common mechanism between generalized obesity and treatment emergent weight gain we do not presume a complete overlap. Some genes like NRXN3 and PAM, both highly significant in the whole-genome association study and each displaying clustering of significant SNPs, were not replicated in the obesity cohort. This is not unexpected nor surprising. Conversely, we did not expect to find all genes underlying the obese phenotype with this study. It is likely that additional genetic predisposition associations for obesity may exist.

Although the above discussion has presented the present invention according to specific methods, systems and apparatus, the present invention has a broader range of applicability. Further, while the foregoing invention has been described in some detail for purposes of clarity and understanding, it will be clear to one skilled in the art from a reading of this disclosure that various changes in form and detail can be made without departing from the true scope of the invention. For example, all the methods, techniques, systems, devices, kits, apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of identifying a treatment-emergent weight gain phenotype, a metabolic syndrome phenotype, an insulin resistance phenotype, or an obesity predisposition phenotype for an organism or biological sample derived therefrom, the method comprising:
detecting, in the organism or biological sample, a polymorphism of a gene or a locus closely linked thereto, the gene encoding a protein selected from: PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, and HSF2, wherein the polymorphism is associated with the treatment-emergent weight gain phenotype, the metabolic syndrome phenotype, the insulin resistance phenotype, or the obesity predisposition phenotype; and,
correlating the polymorphism to the treatment-emergent weight gain phenotype, the metabolic syndrome phenotype, the insulin resistance phenotype, or the obesity predisposition phenotype, thereby identifying the treatment-emergent weight gain phenotype, the metabolic syndrome phenotype, the insulin resistance phenotype, or the obesity predisposition phenotype.

2. The method of claim 1, wherein the metabolic syndrome phenotype comprises insulin resistance or central obesity.

3. The method of claim 1, wherein the treatment-emergent wieght gain phenotype comprises weight gain induced by treatment with an atypical antipsychotic medication.

4. The method of claim 1, wherein the treatment-emergent wieght gain phenotype comprises weight gain induced by olanzapine treatment.

5. The method of claim 1, wherein the organism is a mammal, or the biological sample is derived from a mammal.

6. The method of claim 1, wherein the organism is a human patient, or the biological sample is derived from a human patient.

7. The method of claim 1, wherein the detecting comprises amplifying the polymorphism or a sequence associated therewith and detecting the resulting amplicon.

8. The method of claim 7, wherein the amplifying comprises:
a) admixing an amplification primer or amplification primer pair with a nucleic acid template isolated from the organism or biological sample, wherein the primer or primer pair is complementary or partially complementary to at least a portion of the gene or closely linked polymorphism, or a to proximal sequence thereto, and is capable of initiating nucleic acid polymerization by a polymerase on the nucleic acid template; and,
b) extending the primer or primer pair in a DNA polymerization reaction comprising a polymerase and the template nucleic acid to generate the amplicon.

9. The method of claim 7, wherein the amplicon is detected by a process that includes one or more of: hybridizing the amplicon to an array, digesting the amplicon with a restriction enzyme, or real-time PCR analysis.

10. The method of claim 7, comprising partially or fully sequencing the amplicon.

11. The method of claim 7, wherein the amplifying comprises performing a polymerase chain reaction (PCR), reverse transcriptase PCR (RT-PCR), or ligase chain reaction (LCR) using nucleic acid isolated from the organism or biological sample as a template in the PCR, RT-PCR, or LCR.

12. The method of claim 1, wherein the polymorphism is a SNP.

13. The method of claim 1, wherein the polymorphism comprises an allele selected from the group consisting of those listed in Appendix 1.

14. The method of claim 1, wherein the closely linked locus is about 5 cM or less from the gene.

15. The method of claim 1, wherein correlating the polymorphism comprises referencing a look up table that comprises correlations between alleles of the polymorphism and the phenotype.

16. The method of claim 1, wherein the organism is a non-human mammal and the method further comprises selecting the non-human mammal from a population of non-human mammals, based upon the phenotype.

17. The method of claim 16, comprising breeding the resulting selected non-human mammal with another non-human mammal to optimize the phenotype in one or more offspring.

18. A method of identifying a modulator of a treatment-emergent weight gain phenotype, metabolic syndrome phenotype, an insulin resistance phenotype, or an obesity predisposition phenotype, the method comprising:
contacting a potential modulator to a gene or gene product, wherein the gene or gene product encodes a protein selected from: PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, and HSF2; and,
detecting an effect of the potential modulator on the gene or gene product, thereby identifying whether the potential modulator modulates the treatment-emergent weight gain phenotype, the metabolic syndrome phenotype, the insulin resistance phenotype, or the obesity predisposition phenotype.

19. The method of claim 18, wherein the metabolic syndrome phenotype comprises insulin resistance or central obesity.

20. The method of claim 18, wherein the treatment-emergent wieght gain phenotype comprises weight gain induced by olanzapine treatment.

21. The method of claim 18, wherein the gene or gene product comprises a polymorphism selected from those listed in Appendix 1.

22. The method of claim 18, wherein the effect is selected from:
(a) increased or decreased expression of PAPPA, PAM, pf20, DNAH11, PKD1, KCNMA1, PKHD1, NRXN3, EPHA7, ROS1, FKSG87, C3orf6, TOX, DLG2, MDS1, FABP2, EFA6R, FLJ20125, C1orf10, CHL1, BICD1, KREMEN1, ADARB2, A2BP1, MGC4309, PIGR, PCSK7, or HSF2 in the presence of the modulator;
(b) increased or decreased cleavage of IGFBP4 by PAPPA in the presence of the modulator;
(c) increased or decreased catalysis of peptide cleavage by PAM in the presence of the modulator;
(d) increased or decreased cleavage of IGFBP4 by PAPPA in the presence of the modulator;
(e) increased or decreased catalysis of peptide cleavage by PAM in the presence of the modulator;
(f) change in function of cilia comprising pf20and/or DNAH11 in the presence of the modulator;
(g) change in association (affinity, etc.) of PKD1 gene product, polycystin-1, with PKD2 gene product, polycystin-2 in the presence of the modulator;
(h) change in localization of polycystin-2 in or to a plasma membrane in the presence of the modulator;
(i) change in activity of a channel comprising a polycystin-1 in the presence of the modulator;
(j) change in localization of a KCNMA1 gene product in the presence of the modulator; and,
(k) change in activity of a channel comprising KCNMA1 gene product in the presence of the modulator.

23. A kit for treatment of a treatment-emergent wieght gain phenotype, metabolic syndrome phenotype, an obesity predisposition phenotype or an insulin resistance phenotype, the kit comprising a modulator identified by the method of claim 18 and instructions for administering the compound to a patient to treat the treatment-emergent wieght gain phenotype, the metabolic syndrome phenotype, the obesity predisposition phenotype or the insulin resistance phenotype.

24. The kit of claim 23, wherein the metabolic syndrome phenotype is an obesity predisposition or insulin resistance phenotype.

25. A system for identifying a treatment-emergent wieght gain phenotype, metabolic syndrome phenotype, an insulin resistance phenotype, or an obesity predisposition phenotype for an organism or biological sample derived therefrom, the system comprising:
a) a set of marker probes or primers configured to detect at least one allele of one or more gene or linked locus associated with the metabolic syndrome phenotype, wherein the gene encodes PAPPA, PAM, pf20, DNAH11, PKD1, KCNMA1, PKHD1, NRXN3, EPHA7, ROS1, FKSG87, C3orf6, TOX, DLG2, MDIS 1, FABP2, EFA6R, FLJ20125, C1orf10, CHL1, BICD1, KREMEN1, ADARB2, A2BP1, MGC4309, PIGR, PCSK7, or HSF2;
b) a detector that is configured.to detect one or more signal outputs from the set of marker probes or primers, or an amplicon produced from the set of marker probes or primers, thereby identifying the presence or absence of the allele; and,
c) system instructions that correlate the presence or absence of the allele with the predicted treatment-emergent wieght gain phenotype, metabolic syndrome phenotype, the insulin resistance phenotype, or the obesity predisposition phenotype, thereby identifying the metabolic syndrome phenotype, the insulin resistance phenotype, or the obesity predisposition phenotype for the organism or biological sample derived therefrom.

26. The system of claim 25, wherein the metabolic syndrome phenotype comprises insulin resistance or central obesity.

27. The system of claim 25, wherein the treatment-emergent weight gain phenotype comprises weight gain induced by olanzapine treatment.

28. The system of claim 25, wherein the set of marker probes comprises a nucleotide sequence provided in Appendix 1.

29. The system of claim 25, wherein the detector detects one or more light emission, wherein the light emission is indicative of the presence or absence of the allele.

30. The system of claim 25, wherein the instructions comprise at least one look-up table that includes a correlation between the presence or absence of the allele and the metabolic syndrome, treatment-emergent weight gain, insulin resistance or obesity predisposition.

31. The system of claim 25, wherein the system comprises a sample.

32. The system of claim 31, wherein the sample comprises genomic DNA, amplified genomic DNA, cDNA, amplified cDNA, RNA, or amplified RNA.

33. The system of claim 31, wherein the sample is derived from a mammal.
